(19) United States
(12) Patent Application Publication
(10) Pub. No.: US 2023/0133866 A1
(43) Pub. Date: May 4, 2023

(54) INTEGRATED PHOTONIC SYSTEMS AND METHODS FOR BIOSENSING

(71) Applicant: SiPhox Inc., Cambridge, MA (US)

(72) Inventors: Diedrik Rene Vermeulen, Cambridge, MA (US); Michael Dubrovsky, Cambridge, MA (US); Sarat Gundararapu, Cambridge, MA (US)

(21) Appl. No.: 17/918,102

(22) PCT Filed: Apr. 9, 2021
(86) PCT No.: PCT/US2021/026716

§ 371 Date: Oct. 10, 2022

Related U.S. Application Data
(60) Provisional application No. 63/007,927, filed on Apr. 9, 2020.
(60) Provisional application No. 63/021,251, filed on May 7, 2020.
(60) Provisional application No. 63/065,594, filed on Aug. 14, 2020.
(60) Provisional application No. 63/091,250, filed on Oct. 13, 2020.
(60) Provisional application No. 63/094,861, filed on Oct. 21, 2020.

(51) Int. Cl. G01N 33/543; G01N 1/44; G01N 1/42; G01N 1/38; G01N 1/14; G01N 35/00; G01N 21/01; G01N 33/53

(52) U.S. Cl. CPC G01N 33/54386; G01N 1/44; G01N 1/42; G01N 1/38; G01N 1/14; G01N 35/0099; G01N 21/01; G01N 33/5308; G01N 2201/08; G01N 2035/00346; G01N 2035/00534

(57) ABSTRACT

Disclosed herein are integrated photonics systems (3800) for biosensing including an interrogator photonic circuit (3802) and cartridge (3804) and methods using these systems. The cartridge (3804) comprises a sensor photonic integrated subcircuit. The cartridge (3804) is configured to receive a biological sample. The interrogator photonic circuit (3802) is optically coupled to the cartridge (3804) an comprises: (i) a light source (3806) configured to generate light; and (ii) one or more waveguides configured to carry the light, wherein the light is used to determine a characteristic of the biological sample in the cartridge (3804). A system can have an assembly of a plurality of modular photonic integrated subcircuits. Each subcircuit can be pre-fabricated and can be configured to transfer light to and receive light from another subcircuit based on the first functionality. An output port of a first subset of the subcircuits can be configured to be aligned with an input port of a second subset of the subcircuits. At least one subcircuit can be configured to be removed from the first integrated photonics assembly and connected to a second integrated photonics assembly having a second functionality. The first integrated photonics assembly can be different from the second integrated photonics assembly and the first functionality can be different from the second functionality.

### CROSS-REFERENCE TO RELATED APPLICATIONS

This application claims priority to and the benefit of U.S. Provisional Application No. 63/007,927 titled “PROBE: Pandemic Real-time Optical Biosensing Engine” and filed Apr. 9, 2020, U.S. Provisional Application No. 63/021,251 titled “Means for Adapting Biosensing Chemistries to Waveguide-Based Detection” and filed May 7, 2020, U.S. Provisional Application No. 63/091,250 titled “Laser Based Photonic Biosensors” and filed Oct. 13, 2020, U.S. Provisional Application No. 63/065,594 titled “Design for Tabletop and Handheld Photonics-Based Medical Diagnostics Platform” and filed Aug. 14, 2020, and U.S. Provisional Application No. 63/094,861 titled “Automated Reaction System Coupled to Integrated Photonic Biosensors” and filed Oct. 21, 2020, which are incorporated herein by reference in their entireties.

### TECHNICAL FIELD

The following disclosure is directed to integrated photonic systems and methods for biosensing and, more specifically, integrated photonic systems and methods for real-time or near real-time biosensing, including detecting changes in optical response to biological activity.

### BACKGROUND

The emergence of personalized medicine, global pandemic risks, and other 21st century health trends requires label-free, low-cost diagnostic technology. Cheap disposable tests capable of monitoring multiple disease biomarkers are needed. Existing diagnostic tools employing disposable bio-photonic sensor chips are interrogated via free-space optics (i.e., non-integrated photonics). Diagnostic systems such as these may be effective in some responses but they are typically bulky, inflexible, and expensive.

### SUMMARY

In one aspect, the disclosure features integrated photonic systems for biosensing. An example system can include a cartridge comprising a sensor photonic integrated subcircuit, the cartridge configured to receive a biological sample, and an interrogator photonic circuit optically coupled to the cartridge. The interrogator can include (i) a light source configured to generate light; and (ii) one or more waveguides configured to carry the light, in which the light is used to determine a characteristic of the biological sample in the cartridge.

Various embodiments of the integrated photonic systems for biosensing can include one or more of the following features.

The system can include a stage configured to removably engage the cartridge and facilitate alignment of a light path of the interrogator photonic circuit and a light path of the cartridge. The stage can include an ultrasound or a sound generator for at least one of: (i) preventing non-specific binding or (ii) mixing. The stage can include at least one of: (a) a thermoelectric heater or (b) a thermoelectric cooler. The system can include an isolation window disposed between the cartridge and the interrogator photonic circuit and configured to: (a) physically isolate the interrogator photonic circuit from the biological sample, and (b) enable the light to pass between the interrogator photonic circuit and the cartridge.

The system can include an alignment module configured to facilitate alignment between a light path of the cartridge and a light path of the interrogator photonic circuit. The alignment module can actively facilitate alignment between the light path of the cartridge and the light path of the interrogator photonic circuit. The alignment module can passively facilitate alignment between the light path of the cartridge and the light path of the interrogator photonic circuit. The alignment module can enable an optical coupling efficiency greater than 10%. The system can include an indicator coupled to the alignment module and configured to display a signal indicating whether the cartridge is aligned to the interrogator photonic circuit. The system can include at least one lens configured to focus light between the interrogator photonic circuit and the cartridge.

The interrogator photonic circuit can include a control circuit configured to control the light. The control circuit can include a detection circuit configured to detect the light. The light source can be edge-coupled to the control circuit. The light source can be coupled to the control circuit via an optical fiber. The cartridge can include a microfluidic cell. The microfluidic cell can include at least one of: (a) a magnetic microstirrer, (b) a plasmonic vortex mixer, or (c) a flow-inducing, device. The microfluidic cell can include the magnetic microstirrer, and the system can further include a stage configured to removably engage the cartridge and facilitate alignment of a light path of the interrogator photonic circuit and a light path of the cartridge, in which the stage includes a transmitter configured to power the magnetic microstirrer. The flow-inducing device can be an absorptive pad or a microfluidic capillary pump. The microfluidic cell can include at least one of: (i) a protein, (ii) a reagent, or (iii) a rinsing fluid. The microfluidic cell can include at least one microfluidic channel, in which a wall of the channel has an amplifier enzyme attached thereto.

The stage can be configured to receive a plurality of cartridges. The system can include a splitter coupled to the light source; and a frequency discriminator coupled to the splitter and configured to determine a change in a wavelength of the light source. The frequency discriminator includes an unbalanced Mach-Zehnder interferometer (MZI), a Fabry-Perot cavity, a ring resonator, a gas cell, or a free-space etalon. The frequency discriminator can include at least one of silicon, silica, or silicon nitride. The light source can be tunable thermally, electrically, and/or mechanically. The system can include a robotic device coupled to the interrogator photonic circuit and configured to position the cartridge to contact the biological sample. The robotic device can be configured to discard the cartridge. The robotic device can be configured to replace the cartridge automatically.

In another aspect, the disclosure features methods for biosensing. An example method can include obtaining a biological sample in a cartridge, in which the cartridge includes a sensor photonic integrated subcircuit. The method can include positioning the cartridge relative to an interrogator photonic circuit such that the cartridge is optically coupled with the interrogator photonic circuit, in which the interrogator photonic circuit includes (i) a light source configured to generate light, (ii) a waveguide configured to carry the light, and iii) a photodeteaor configured to detect said light after passing through said waveguides; and determining, via the light, a characteristic of the biological sample in the cartridge.

Various embodiments of the biosensing methods can include one or more of the following features.

The method can include determining, via an alignment module, whether the cartridge is optically coupled with the interrogator photonic circuit. The method can include determining a coupling efficiency between the cartridge and the interrogator. The characteristic of the biological sample can be determined based on a change in resonance, interference, or absorption caused by the biological sample. The waveguide can be optically coupled to a probe. The probe can bind specifically to a target biomolecule in the sample. The probe can be an antibody, an antigen, or an aptamer. The target biomolecules can be bound by a detection antibody. The detection antibody can include an optically active component. The component of the biological sample can initiate a cleavage of said probe. The probe can include an optically active component. The optically active component can be a plasmonic nanoparticle, a gold nanoparticle, a quantum dot, or a fluorophore. The probe can include a silicon particle.

The probe can include a magnetic particle. The magnetic particle can include iron-oxide. The waveguide can include an optical ring resonator or an unbalanced Mach-Zehnder interferometer. The component of the biological sample can activate a cleaving component. The component of the biological sample can bind to a hairpin RNA encoding a cleaving component, in which the binding can facilitate translation of the RNA to generate said cleaving component. The cleaving component can be a CRISPR enzyme. The cartridge can further include an electromagnet. A target biomolecule of the sample can be functionalized with a magnetic particle.

In another aspect, the disclosure features methods for detecting a target biomolecule in a biological sample. An example method can include providing a device comprising a sensor functionalized with a probe. The probe can be cleaved by a cleavage enzyme. The method can include adding the biological sample to the device, in which the presence of said target biomolecule results in generation of or activation of said cleavage enzyme. The method can include detecting cleavage of the probe by said cleavage enzyme, thereby detecting the presence of the target biomolecule in the biological sample. The cleavage enzyme can be a CRISPR complex. The CRISPR complex can be a Cas12 complex or a Cas13 complex. The target biomolecule can be RNA or DNA. The target biomolecule can bind to a hairpin RNA encoding the cleavage enzyme, in which the binding facilitates translation of said hairpin RNA to generate the cleavage enzyme. The sensor can be an electrical sensor, an optical sensor, or a combination thereof.

The sensor can include a ring resonator or a Mach-Zehnder interferometer. The probe can include an optically active component. The optically active component can be a plasmonic nanoparticle, a gold nanoparticle, a quantum dot, or a fluorophore. The probe can include a silicon particle. The probe can include a magnetic particle. The magnetic particle can include iron-oxide.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the systems and methods described herein. In the following description, various embodiments are described with reference to the following drawings.

FIG. 1 is a diagram of a perspective view of an example integrated photonics assembly that multiple photonic integrated subcircuits.

FIG. 2 is a diagram of a top view illustrating light transfer between example subcircuits of an integrated photonics assembly.

FIGS. 3A-3C are diagrams of top views of example integrated photonics assemblies, which each include multiple subcircuits.

FIG. 4 is a diagram of a top view of an example packaged 1D integrated photonics assembly.

FIG. 5 is a diagram of a top view of an example packaged pseudo-2D integrated photonics assembly.

FIG. 6 is a diagram of a top view of an example packaged integrated photonics assembly formed in the shape of a closed-loop “snake”.

FIG. 7 is a diagram of a top view of an example packaged integrated photonics assembly formed in the shape of an open-loop “snake”.

FIG. 8 is a diagram of a top view of an example packaged assembly, illustrating that the subcircuits can be standardized.

FIG. 9 is a diagram of a top view of an example integrated photonics assembly formed into a “checker” type assembly.

FIG. 10A is a diagram of a top view of an example ID integrated photonics assembly.

FIGS. 10B-10C are diagrams of top views of a representative subcircuit of the assembly of FIG. 10A.

FIGS. 11A-11B are diagrams of top views of alternative examples of the assembly of FIG. 10A.

FIGS. 12A-12B are diagrams of top views of example assemblies of four integrated photonic subcircuits, in which each subcircuit is configured to transfer light to an adjacent subcircuit.

FIGS. 13A-13B are diagrams of top views of four example integrated photonic subcircuits, in which each subcircuit is configured to transfer light to an adjacent subcircuit.

FIGS. 14A-14B are diagrams of top views of example assemblies including four subcircuits each.

FIGS. 15A-15B are diagrams of top views of examples photonic monitoring circuits for photonic integrated subcircuits.

FIG. 16A is a diagram of a top view of an example 1D integrated photonics assembly including three subcircuits, in which each subcircuit has at least one monitoring circuit and a useful circuit.

FIG. 16B is a diagram of a top view of a simplified representation of FIG. 16A to illustrate an example of wavelength dependence of the interfaces between the subcircuits.

FIG. 17 is a diagram of a top view of an example 1D integrated photonics assembly including four subcircuits.

FIG. 18 is a diagram of a top view of an example embodiment of a subcircuit.

FIG. 19 is a diagram of a top view of an example receptacle configured to be complementary to the subcircuit of FIG. 18 and configured to align two subcircuits of FIG. 18.

FIG. 20 is a diagram of a top view of multiple subcircuits of FIG. 18A positioned on the receptacle of FIG. 19.

FIG. 21 is a diagram of a top view of an example subcircuit including photonic circuit and input and output waveguides.

FIG. 22 is a diagram of a top view of an example connector chip that may be used in assembling two subcircuits.

FIG. 23 is a diagram of a top view of an example assembly of subcircuits.

FIGS. 24A-24D are diagrams of top views of example variations of subcircuits.

FIG. 25A is a diagram of a top view of an example receptacle configured to receive subcircuits of FIGS. 24A-24D. FIG. 25B is a diagram of a top view of the receptacle of FIG. 25A connected to four subcircuits of FIG. 24A.

FIG. 26A is a diagram of a top view of an example receptacle configured to receive subcircuit of FIG. 24A. FIG. 26B is a diagram of a top view of an example receptacle connection to four subcircuits of FIG. 24A.

FIG. 27 is a diagram of a cross-sectional view of an example subcircuit.

FIG. 28 is a diagram of a cross-sectional view of the subcircuit of FIG. 27 in combination with the receptacle.

FIG. 29 is a diagram of a cross-sectional view of an example receptacle.

FIG. 30 is a diagram of a top view of example subcircuits aligned to an example receptacle.

FIGS. 31A-31D are diagrams of cross-sectional views of example fabrication steps for fabricating a receptacle wafer. FIG. 31E is a diagram of a top view of FIG. 31D.

FIGS. 32A-32E are diagrams of cross-sectional views of an example alternative method to fabricate a receptacle.

FIGS. 33A-33C are diagrams of cross-sectional views of an example method to fabricate the receptacle directly on a silicon wafer.

FIG. 34 is a diagram of a perspective view of an example 3D drawing of a subcircuit having shallow-etched vertical alignment features and deep-etched lateral alignment features.

FIG. 35 is a diagram of a top view of an example receptacle wafer including example assemblies of subcircuits.

FIG. 36 is a diagram of a cross-sectional view of portions of a receptacle and portions of subcircuits for illustrating a method for elastic averaging.

FIG. 37 is a flowchart of a method for aligning two or more subcircuits to a receptacle.

FIG. 38 is a diagram of an integrated photonic system for biosensing including an interrogator and cartridge.

FIG. 39 is a flowchart of a method for biosensing utilizing the integrated photonic system.

FIG. 40 is a diagram of a packaged integrated photonic system for biosensing.

FIGS. 41-42 are diagrams of embodiments of an interrogator.

FIGS. 43A-43C are diagrams of embodiments of an alignment module.

FIG. 44 is a diagram of an embodiment of a stage, which enables the efficient and easy replacement and/or alignment of the cartridge.

FIG. 45 is a diagram of an example cartridge, which may be an assembly including a sensor chiplet and one or more microfluidic cells.

FIG. 46 is a perspective rendering of various components associated with the photonic biosensing platform.

FIG. 47 is a perspective rendering of the example tabletop apparatus of FIG. 46, including a display, disposable test cartridges, apparatus input ports, and buttons.

FIG. 48 is a perspective rendering of the example portable apparatus of FIG. 46, and related components, and the display and related software of FIG. 46.

FIG. 49 is a perspective rendering of the example handheld apparatus of FIG. 46 optically coupled to a cartridge.

FIG. 50 is a close-up view of example cartridge of FIG. 47 configured to be inserted into the tabletop apparatus or handheld apparatus.

FIGS. 51A-51D are renderings of four implementations of a biosensor chip with microfluidics that may be used within a test cartridge.

FIG. 52A is a diagram of the sensor chiplet having a waveguide including antibodies in at least one channel.

FIG. 52B is a diagram of a Mach-Zender Interferometer (MZI)-type sensor.

FIG. 53A is a diagram of an example method where an antigen binds to an antibody immobilized on a ring resonator.

FIG. 53B is a diagram of an example process wherein the cleaving agent cleaves a reporter probe from a waveguide.

FIG. 54 is a diagram of an example testing mechanism used by the biosensing apparatus.

FIG. 55 is a diagram of a cleaving component configured to be activated when it detects a sensing target of interest.

FIG. 56 is a diagram illustrating the example target-specific cleavage process.

FIG. 57 is a diagram of an example method for RNA detection using a toehold switch RNA approach.

FIG. 58 is a diagram of an example method for DNA and RNA detection using CRISPR and a waveguide.

FIG. 59 is a diagram of an example method attaching a magnetic particle to a sensing target may direct the sensing target to the waveguide via an applied magnetic field.

FIG. 60 is a diagram of an example microfluidic channel that transports analyte to the waveguide.

FIG. 61 is a diagram of an example implementation of the multi-photonic-chiplet (MPC)-based optical biosensor assembly for multiplexed sensing of analytes.

FIG. 62 is a diagram of an example layout of the photonic biosensor with a single sensing element.

FIG. 63A is a diagram of an examples system including the sensing element(s) and the frequency discriminator.

FIGS. 63B-63C, is a plot illustrating the functionality of the sensing elements and frequency discriminator of FIG. 63A.

FIG. 64 is a diagram of an example implementation of an automated biosensing system capable of multiple tests within a multi-well plate.

FIG. 65 is a diagram of mechanical alignment between a robot arm and a disposable sensor tip to enable optical communication between the reusable arm having reusable optical components and the disposable sensor chiplets on the sensor tips.

FIG. 66 is a perspective rendering of an example implementation of automated liquid handling to insert sample tests directly into the disposable sensors coupled to a tabletop biosensing platform.

FIG. 67 is a diagram of an example implementation of an automated liquid handling system to draw samples into tubes and flow them over sensor chips via microfluidic channels.

FIG. 68 is a diagram of an example sensor chip with edge facets of waveguides used for biolayer interferometry.

### DETAILED DESCRIPTION

Disclosed herein are embodiments of photonic integrated subcircuits that can be assembled into an integrated photonics assembly. These photonic integrated subcircuits may be referred to herein as “subcircuits”, “chiplets”, or “sub-chips”. The integrated photonics assembly may he referred to herein as “an assembly”, “an integrated photonics assembly”, or “a photonic integrated circuit” (PIC). A given photonic integrated subcircuit can be configured to transfer light to and/or receive light from at least one other subcircuit, for example, using one or more light transfer techniques. In various embodiments, each photonic integrated subcircuit is a discrete integrated circuit or chip that be physically separated from one another, moved, and/or attached to one another. The example subcircuits can be assembled to create a larger integrated photonics circuit using two or more subcircuits. The example subcircuits may be used to extend and/or combine an integrated photonic circuit into a larger integrated photonic circuit. The example subcircuits are configured to guide light via waveguide structures and may contain special functions including, e.g., splitting light, wavelength demultiplexing, photo detection, light generation, light amplification, etc.

### Standardization of Photonic Integrated Subcircuits

In various embodiments, each subcircuit is a pre-fabricated integrated circuit. By pre-fabricating the subcircuits, the subcircuits can be standardized so as to enable assembly of two or more subcircuits into a PIC. As discussed further herein, standardization of subcircuits can pertain to one or more properties of the subcircuits, including dimension(s), volume, weight, input(s), output(s), functionality, mechanical feature(s) (e.g., for coupling, alignment, etc.), active alignment feature(s), wirebond pad(s), electrical connection(s), feature(s) that are complementary to a receptacle (including vertical alignment features) and/or lateral alignment features), etc. Standardization can include the configuration of complementary properties or structures of two or more adjacent subcircuits, as described further below. For instance, alignment structures and/or waveguide paths in a first type of subcircuit may be configured to be complementary with respective alignment structures and/or waveguide paths in a second type of subcircuit, such that a subcircuit of a first type can be attached to a subcircuit of a second type, e.g., with low optical loss. Standardization of the subcircuits can enable permutational assembly of the subcircuits into PICS. Further, standardization can enable time-efficient and/or cost-efficient packaging.

Because many different types of integrated photonics assembly can be created from the subcircuits, it is beneficial to standardize the subcircuits. One benefit of standardization is that a subcircuit can be switched or interchanged with another subcircuit, thereby creating a different optical assembly that is a variation of the first assembly. In some cases, subcircuits can be configured such that they enable many optical assemblies that are useful with a minimum number of subcircuits. Further, each subcircuit or type of subcircuit can be configured and/or selected for improved performance, reduced cost, efficient or ease of fabrication, efficient or ease of supply, etc.

Note that there is a nonzero likelihood that certain aspects and/or components (e.g., transistors) of an integrated circuit may fail or render the individual fabricated circuit defective. The resulting integrated circuits of a particular fabricated batch that function correctly is the “yield” of that particular batch. By fabricating (and subsequently testing) the integrated photonics subcircuits individually and/or independently, the non-functioning subcircuits can be eliminated from the supply of subcircuits. Further, it is found that a higher number of functioning subcircuits (of a given type) can be produced using a single type of fabrication process (e.g., on a given wafer). In comparison, a mixed-type integrated circuit (e.g., using more than one type of fabrication process) results in lower yield of that mixed-type integrated circuit. This results in a higher number of fully-functioning integrated subcircuits, thereby contributing to an increased number of integrated photonics assemblies. Therefore, in some cases, it may be preferrable to generate an integrated optical circuit from subcircuits even if all the component subcircuits can be fabricated in the same process. This can increase the number of optical assemblies that can be built. Furthermore, the subcircuits can be yielded before they are used in the optical assembly, thereby increasing the total yield of a certain optical assembly. The optical assembly can thus be yield-optimized by forming the assembly from different sub-chips.

In some embodiments, yields are significantly improved in an integrated photonics assembly as compared to a monolithic chip. In some embodiments, cost is significantly reduced in an integrated photonics assembly as compared to a monolithic chip. As illustrated below, improvements in yield and/or cost may depend on the type of internal component or functionality. The following tables provide two numerical examples comparing the yields of traditional “monolithic” integrated photonic circuits to the yields of the modular integrated photonics assemblies, as described herein. In particular, the left side of Table 1 illustrates a monolithic chip that is fabricated with two wavelength demultiplexers (WDMs) in which each individual WDM typically has a 50% yield. Further, the right side of Table 1 illustrates a modular assembly including two 50%-yield WDMs. As illustrated, even with the cost of assembly, the total cost of the assembly is significantly less (e.g., at least 55% less) than the total cost of a monolithic chip.

TABLE 1
Yield comparison between a monolithic chip and
a modular assembly having two 50%-yield WDMs.

| | |
|---|---|
| Monolithic Chip having | Modular Assembly having |
| two 50%-yield WDMs | two 50%-yield WDMs |

| | | | |
|---|---|---|---|
| Cost | $10 | Cost | $10 |
| Yield | 25% | Yield | 50% |
| True Cost | $40 | True Cost | $20 |
| | | Total Cost = True | $22 |
| | | Cost + Assembly Cost | |

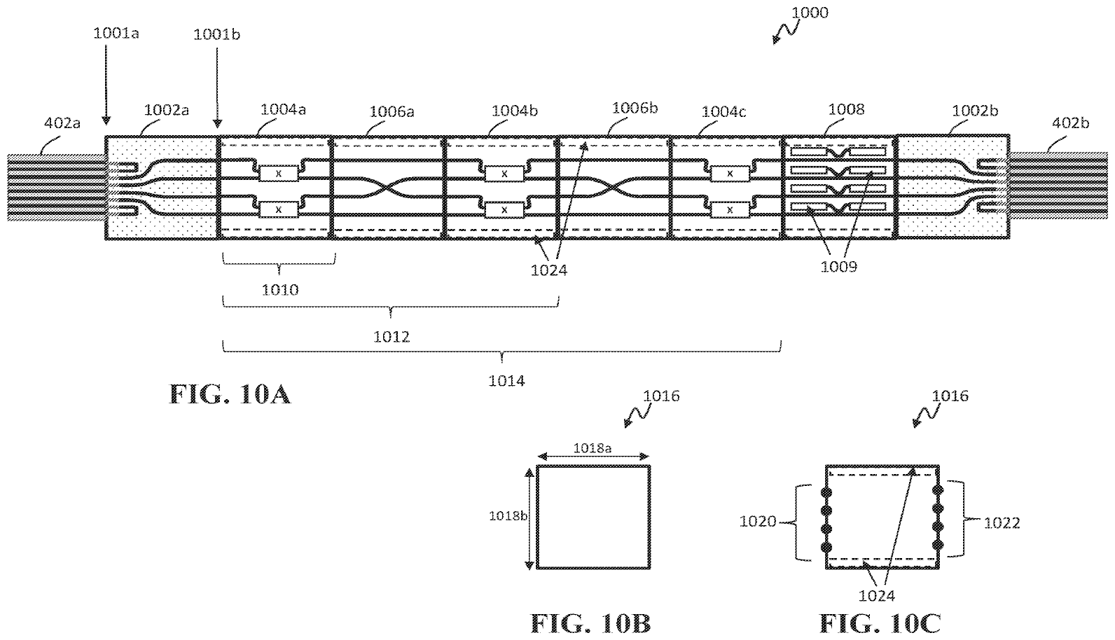

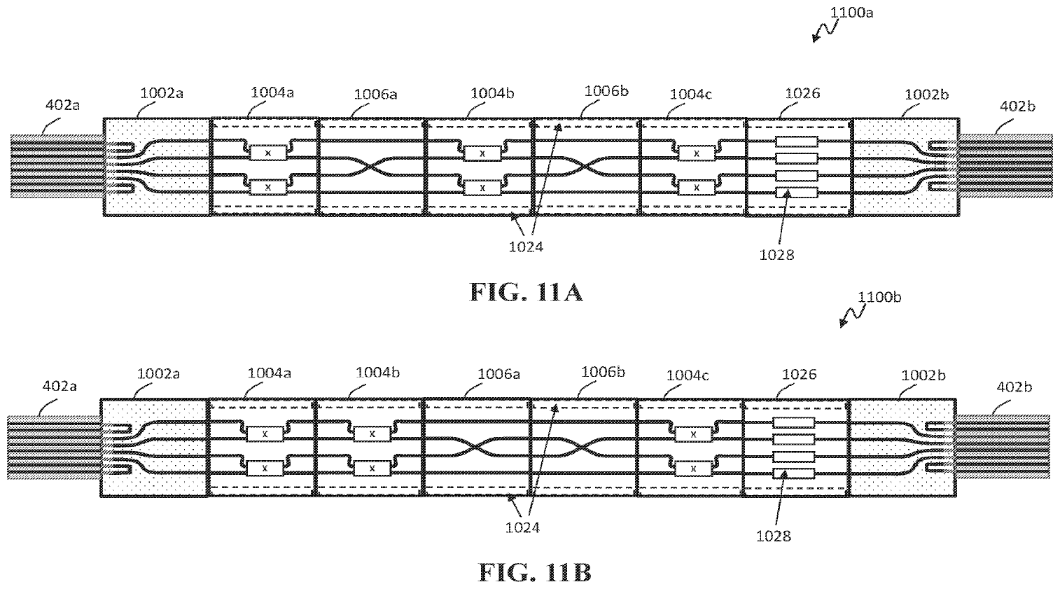

Another illustration of the yield difference and cost is provided in Table 2 below. Both yield and cost are dramatically improved for the modular assembly over the monolithic chip. Refer to FIG. 10A for an example of an integrated photonics assembly 1000 including a subcircuit 1008 having Ge photodetectors (PDs).

TABLE 2
Yield comparison between a monolithic chip and
a modular assembly having two 90%-yield PDs.

| | |
|---|---|
| Monolithic Chip having | Modular Assembly having 100 |
| 100 98%-yield Ge PDs | 98%-yield Ge PDs |

| | | | |
|---|---|---|---|
| Cost | $20 | Cost | $20 |
| Yield | 13% | Yield | 81.7% |
| True Cost | $154 | True Cost | $25 |
| | | Total Cost = True | $27 |
| | | Cost + Assembly Cost | |

In some embodiments, subcircuits are standardized in size. For example, a standardized set of subcircuits may include subcircuits that are each 1 mm in width and 1 mm in length. In some cases, the standardized set may include two or more subsets of subcircuits in which the size of subcircuits in each subset is standardized. For example, a first subset may have subcircuits of 1 mm×1 mm, a second subset of subcircuits of 1 mm×2 mm, a third subset of subcircuits of 2 mm×2 mm, a fourth subset of subcircuits 1 mm×3 mm, etc.

In some embodiments, the subcircuits are standardized according to the light port positioning and/or electrical pad positioning. For instance, the position of light input ports and/or output ports along the edges or surface of the subcircuits may be standardized for groups of subcircuits. By leveraging standardization, a library of standard subcircuits can be produced to build nearly an endless variety of photonic assemblies without the need for costly or time-consuming customization of the package or assembly process.

In some embodiments, the standardization of subcircuits contributes to and/or directly beget the standardization of other components, e.g., printed circuit boards (PCBs), non-optical components, lasers, etc. For example, by standardizing the electrical pads in a subcircuit, connecting pads on a host PCB can also be standardized, thereby contributing greater efficiency.

### Modularity of Photonic Integrated Subcircuits

Importantly, each subcircuit is configured to be a modular component of an integrated photonics assembly. The modular character of the subcircuits is one benefit of the standardization of the subcircuits. For instance, two or more subcircuits, e.g., subcircuits S₁ and S₂, can be assembled into assembly A with functionality F_{A}. One or more of these subcircuits (e.g., subcircuit S₂) can be removed from assembly A and connected to another subcircuit (e.g., subcircuit S₃) and/or an assembly to form assembly B, in which assembly B has a functionality F_{B} (which may be different from functionality F_{A}). In doing so, the modular character of the subcircuits enable many useful integrated optical assemblies.

Various benefits flow from the modularity of the photonic integrated subcircuits. In particular, the modularity of the subcircuits facilitate the scaling (e.g., scaling up or down) of integrated photonics assemblies, replacement of subcircuits of an assembly, improvements to existing PICs, reconfigurability of assemblies, etc. Importantly, the described systems and methods can produce the desired subcircuits and/or customized integrated photonics assemblies faster than the fabrication of a conventional PIC. For example, a customized integrated photonics assembly may be produced within seven (7) days as compared to the one (1) year required for the conventional PIC. Accordingly, the described systems and methods enable efficiencies in time and/or cost.

Further, the modular subcircuits can reduce waste. For example, as described below, the described systems and methods permit the reuse of existing subcircuits and/or reconfiguring of existing assemblies. In another example, the described techniques enable the fabrication of subcircuits on demand (and therefore a reduction of inventory).

In some embodiments, in a given assembly, a particular subcircuit S is discovered to be faulty (e.g., inefficient, inoperable, incompatible, etc.). That particular subcircuit S may be removed from the assembly and a replacement subcircuit S′ may be installed in its place. In another example, the particular subcircuit S may need to reconfigured and/or translated to another portion of the assembly to be operable. This has the advantage of avoiding disturbing the rest of the assembly while providing a quick and/or simple solution to replacing a faulty part of the assembly. By contrast, a conventional PIC—which requires a single indivisible “chip”—may not be repairable by swapping out or reconfiguring of a fault component.

In another embodiment, the modularity of the subcircuits facilitate the evolution of engineering and/or design of integrated photonics assemblies over time. The development of an assembly A having a particular functionality may change from a first generation (e.g., assembly A₁) configuration to a second generation (assembly A₂), third generation (assembly A₃), and so on to accommodate needs of customers and/or adapt to changing markets, new technologies, different materials, different standards, a change in specifications, evolving regulation, etc. This may be achieved by adding, replacing, moving, reconfiguring, etc. one or more subcircuits in the assembly (e.g., assembly A₁) to produce another assembly (e.g., assembly A₃). For example, at some time after the production of the first generation assembly A₁, a new subcircuit may become available. This new subcircuit may be added to or replace an existing subcircuit in the first generation assembly A₁ to form the second generation assembly A₂.

In another embodiment, an existing assembly A may be repurposed or adapted with a different functionality by changing one or more subcircuits included in the assembly A. In another example, a conventional PIC may be repurposed or reconfigured with a different functionality by adding one or more subcircuits to the PIC. In such a case, an adapter-type subcircuit may be coupled to the conventional PIC and one or more subcircuits may be coupled to the adapter-type subcircuit. In another embodiment, two or more assemblies may be coupled together by one or more subcircuits, e.g., forming a light path between the two or more assemblies.

One primary characteristic of an integrated photonics chip (or subchip) is its ability to guide light. In various embodiments, the subcircuits can be fabricated from one or more electro-optic crystals, polymers, and/or semiconductor materials. For example, this can be achieved in a CMOS-compatible sub-chip or so-called silicon photonics, silicon-on-silica, silicon nitride, aluminum oxide, glass, MTV based integrated photonics chips, lithium niobate, silicon-on-insulator, gallium arsenide (GaAs), indium phosphide (InP), nitride, glass, etc. In some embodiments, the subcircuit is a combination of subcircuits. For example, a silicon photonics subcircuit can be enhanced with a III/V chip to increase its functionality (e.g., optical detection and optical gain), thereby creating a subcircuit that includes two or more chips or subchips.

The example integrated photonics assemblies may be configured for one or more functionalities. The assemblies may be configured for communication, biomedical, chemical, research, computing, or other applications. A non-limiting list of applications include beamforming, beam-steering, LiDAR, biomedical instrumentation (OCT, spectrometers, diagnostics, etc.), biophotonics (blood analysis, brain control, etc.), acousto-optics, astrophotonics, gyroscopes, metrology, optical clocks, magneto-optics (integrated magneto-optical devices, isolators, memory, switches, etc.), artificial intelligence, reconfigurable photonic processors, THz photonics, microwave photonics, fiber sensor interrogators, free-space optical communication (Li-Fi, satellite Internet, etc.), augmented reality, quantum optics (QKD, QRNG, etc.), etc.

### Light Transfer Techniques

Light may be transferred and/or received between two or more subcircuits using one or more light transfer methods, as described in further detail below. Each subcircuit can transfer light to at least one other subcircuit. In some cases, electrical signals, microwave signals, and/or fluids may be transferred and/or received by the subcircuits. In various embodiments, the wavelength of the light can span from 100 nm to 20 microns. Light can be transferred and/or received over one or more channels. In some embodiments, a given channel transmits light in one or more wavelengths, one or more polarizations, and/or one or more modes.

In various embodiments, a subcircuit can be as close as zero (0) micron distance edge-to-edge with another subcircuit. This can be true when two or more subcircuits are stacked horizontally, stacked vertically, or configured to be partially overlapping (e.g., negative distance edge to edge). In various embodiments, the maximum distance between light-transferring subcircuits can be as large as 10 cm. In some embodiments, the distance is between is 0 um and 2 mm.

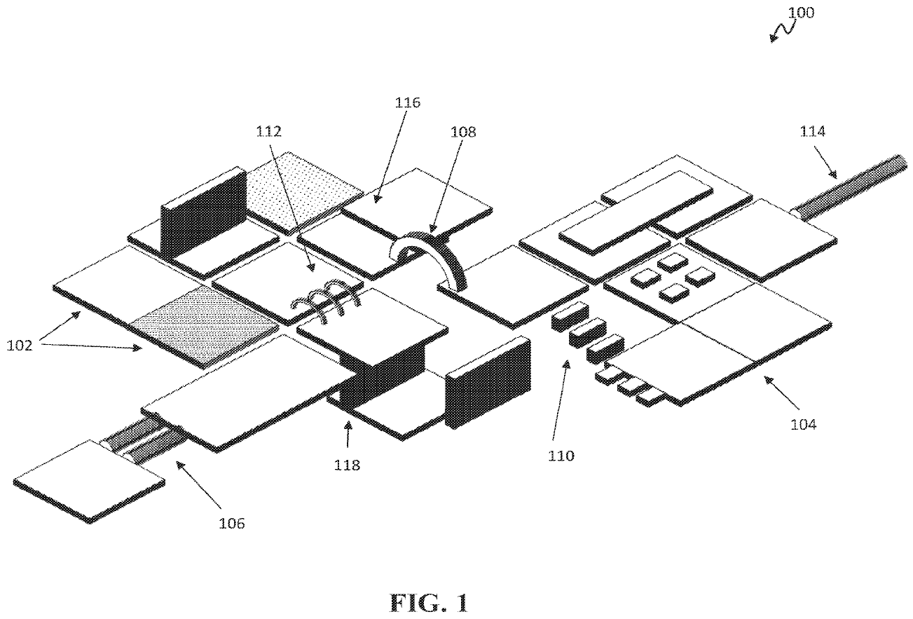

In various embodiments, an integrated photonics assembly can include two or more photonic integrated subcircuits. FIG. 1 illustrates an example integrated photonics assembly 100 that includes multiple subcircuits 102. As depicted, the subcircuits 102 can be coupled to one another by one or more techniques. For example, light can be transferring between two or more subcircuits via butt-coupling 104, optical fiber(s) 106, photonic wirebond(s) 108, a free-space optical train 110, electrical wirebonds 112, adiabatic coupling, out-of-plane coupling, etc. In various embodiments, the integrated photonics assembly 100 can be optically connected to an external system (e.g., a subcircuit, another assembly, a conventional PIC, an electrical system, a computing system, a biomedical system, etc.) by an optical fiber 114. In various embodiments, a channel between two subcircuits can transfer light of one or more polarizations, one or more modes, and/or one or more wavelengths.

The example subcircuits may be arranged in various configurations, e.g., side by side, overlapping, etc. For example, one or more subcircuits can be connected on top of, under, or to the side of a host subcircuit. In some embodiments, a host-type subcircuit is larger in at least one dimension than at least one other type of subcircuit so as to provide sufficient space to “carry” a number of subcircuits. In some embodiments, a host-type subcircuits is smaller in at least one dimension than at least one other type of subcircuit so as to act as a “bridge” between two or more subcircuits. Note that, in the drawings, some subcircuits are distinguished by different patterned or colored surfaces to indicate different types or functionalities.

Light transfer can be accomplished by any one or more of the following techniques. In some embodiments, light is transferred by edge-to-edge coupling (also referred to as butt-coupling) between two or more subcircuits (refer to arrow 104). In this technique, light abruptly exits the subcircuit (e.g. via the end of a light path, waveguide, from an output port, etc.) from one side or edge of the subcircuit into air or any other bulk medium. Light can enter abruptly into the side or edge (e.g., via the beginning of a light path, waveguide, into an input port, etc.) of another subcircuit.

In some embodiments, light is adiabatically transferred between subcircuits by a taper system or method. In this technique, two subcircuits are configured to overlap at least partially (refer to arrow 116). In at least one of the subcircuits, the geometry of a waveguide can be configured such that light can be transferred adiabatically or near-adiabatically to another subcircuit.

In some embodiments, light is transferred between subcircuits via an optical guiding medium. Such optical guiding mediums can include an optical fiber 106, a polymer waveguide, a polymer fiber, etc. The light may be guided in the region or space between the subcircuits and may therefore bridge a larger distance with lower optical loss (as compared two subcircuits without the optical guiding medium). In some embodiments, light is transferred in free-space or in a medium via a crossing lens, a collimator, etc.

In some embodiments, light is configured to exit a subcircuit non-horizontally (e.g., near-vertically or vertically) and enter non-horizontally into another subcircuit. In one example, integrated mirrors or grating couplers can be used to accomplish this type of light transfer. In some embodiments, light exits one subcircuit non-horizontally and enter another subcircuit horizontally. In one example, this is achieved by a subcircuit standing vertically on the surface of another sub-chip (illustrated by arrow 118).

The transfer of light between two or more subcircuits can involve any one or combination of the above-described light transfer methods. In some cases, light transfer can two or more methods (or combinations of methods) for two or more respective channels. Using two or more methods of transferring light can be particularly useful in some cases. In one scenario, butt-coupling of subcircuits may be preferred but a particular routing or direction of the light transfer path may be difficult or may require customization. Such a routing can be achieved by using a flexible connection, e.g., a polymer waveguide or a photonic wirebond. In some instances, some subchips may not be identically sized or shaped due to imperfect dicing or cleaving. Therefore, gaps between such subchips can be spanned using a flexible interconnection method.

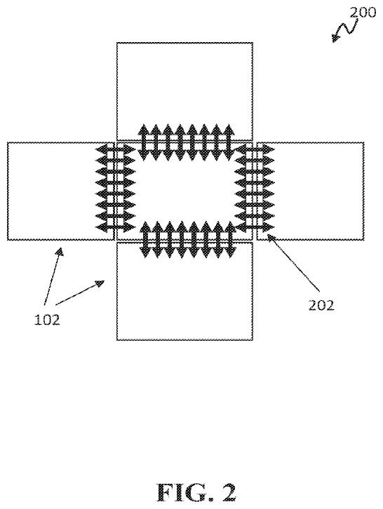

In some embodiments, transfer of light between subcircuits is multi-channel. One benefit of subcircuits that are closely spaced is that many light transfers can happen between the two subcircuits at the same time. As an example, a single subcircuits can transfer light to 10 or more other subcircuits with 100 light channels between each sub-chip. Other free-space components may be added in between the subcircuits and in between the optical path(s). FIG. 2 illustrates light transfer between subcircuits of assembly 200. The assembly 200 includes five (5) subcircuits 102, among which light is transferred and/or received. In the illustrated example, the subcircuits are butt-coupled, thereby making a large number of light transfer paths 202 feasible.

In some embodiments, some chips do not transmit light to a subcircuit and therefore be referred to as “non-photonic subcircuits” or “non-photonic subchips”. For instance, such non-photonic subchips may only transmit and/or receive electrical signals from a photonic assembly of subcircuits. Accordingly, these may not be considered a part of the integrated photonics assembly, However, in some embodiments, these non-photonic subchips are part of a standardized package around the integrated photonics assembly.

In various embodiments, light can be transmitted from the integrated photonics assembly to an external or remote device or system. In some cases, this light may eventually reach other optical chips, though these other chips may not be considered part of the optical assembly. Subcircuits may have light paths to an external system by, for example, a fiber, fiber array or free-space connection. There is no lower bound or upper bound on the number of subcircuits that need to be connected from the assembly to outside world (e.g., an external system or device) and no limitation on which method is used.

### Integrated Photonics Assemblies

As described above, subcircuits can be combined in many different assemblies and configurations. Subcircuits may be combined in a one-dimensional, two-dimensional, or three-dimensional assembly using any one or more of the techniques described herein.

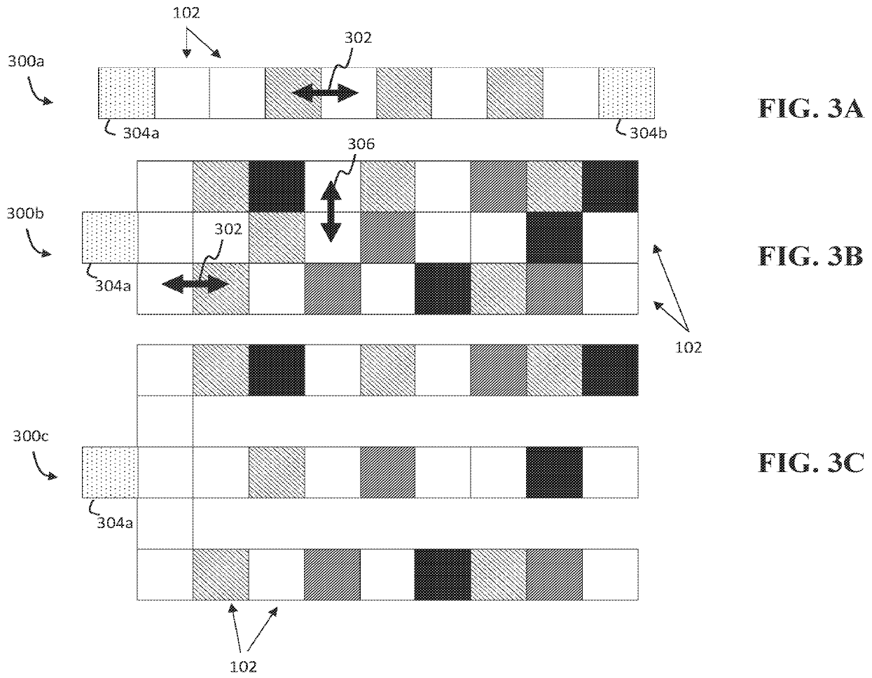

FIGS. 3A-3C provide examples of integrated photonics assemblies, which each include multiple subcircuits 102. In particular, FIGS. 3A-3C illustrate the modularity properties of the subcircuits, including how the subcircuits can be arranged (e.g., coupled, connected, stacked, etc.) and how the photonics assembly can be standardized. Note that, in these examples, the subcircuits are configured to be the same size (in at least two dimensions) and shape.

FIG. 3A illustrates a one-dimensional (1D) array 300a (also referred to as 1D-stacking). In this case, light can be transferred left or right (indicated by arrow 302 and may be referred to as west or east) between at least a subset of the subcircuits 102. The array 300a may begin with a subcircuit 304a and/or end with a subcircuit 304b. In some cases, subcircuits 304aand/or 304b may be able to transfer light to one other subcircuit and/or from one edge of the subcircuit. To enable efficient light transfer between two or more subcircuits 102, the position of the light path within the subcircuits can be standardized to increase assembly permutations, as discussed in more detail herein.

FIG. 3B illustrates an example two-dimensional (2D) array 300b of subcircuits, which includes subcircuits configured with light transfer paths oriented up and down (indicated by arrow 306 and referred to as north and south). FIG. 3C illustrates an example “pseudo” 2D array 300c, which can be considered an extension of the 1D array. The example array 300c enables multiple parallel circuits to be connected together without requiring north and south light transfer capability on most subcircuits.

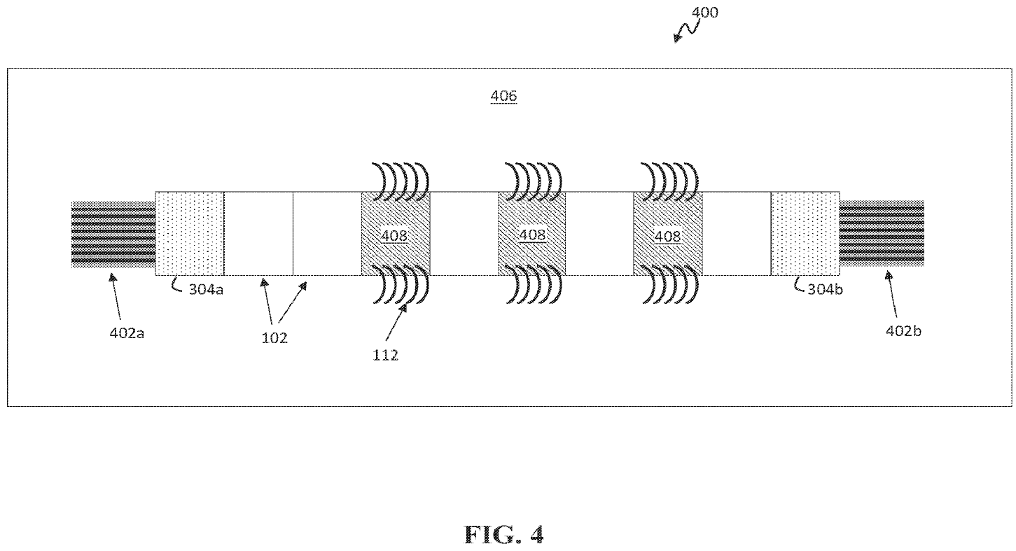

FIG. 4 illustrates an example of a packaged 1D integrated photonics assembly 400. The assembly 400 includes multiple subcircuits 102, a first fiber array 402a connected to the first subcircuit 304a, and a second fiber array 402b connected to the last subcircuit 304b. Note that a subset of the subcircuits are wirebonded via electrical conductors 112 to the printed circuit board (PCB) 406. Wirebonds 112 can be created during the fabrication and/or assembly process. The electrical wirebonds 112 may be standardized such that they can be connected to a particular type of subcircuit 408. Such subcircuits 408 may be configured to handle both light and electrical current.

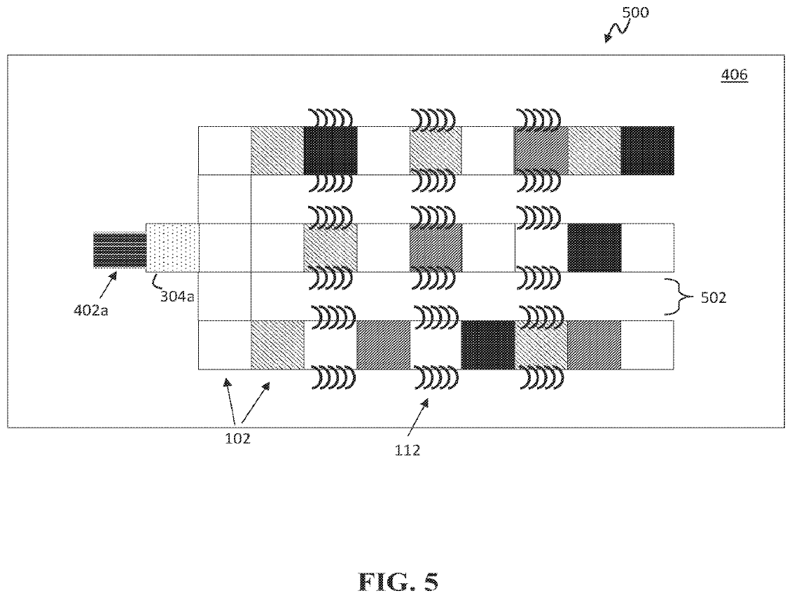

FIG. 5 shows an example of a packaged pseudo-2D integrated photonics assembly 500. A fiber array 402a is connected to the first subcircuit 304a. In this example, because there are empty spaces 502. between parallel rows of subcircuits, the subcircuits are accessibly wirebonded via wirebonds 404 to the PCB 406. Note that the empty spaces 502 can contribute to the standardization of the host PCB by providing space for electrical pads on the PCB via the empty spaces 502.

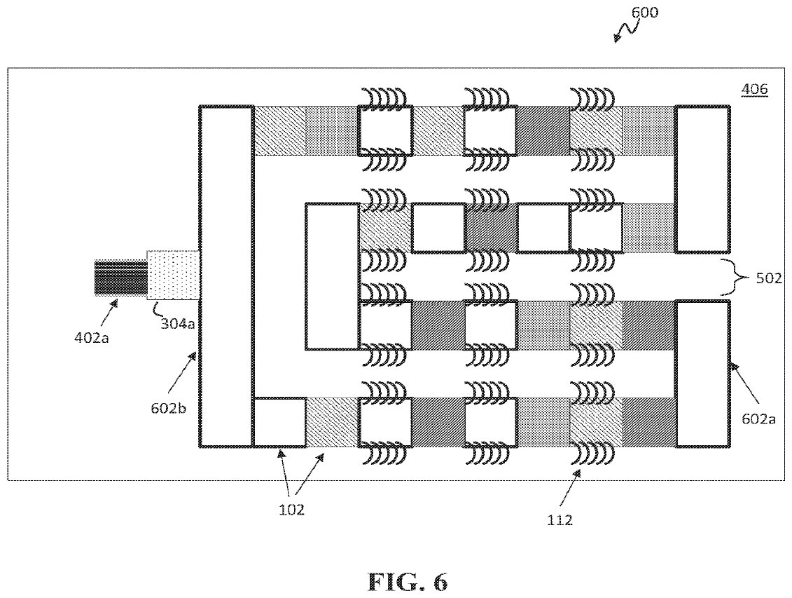

FIG. 6 shows an example of a packaged integrated photonics assembly 600 which is formed in the shape of a closed-loop “snake”. In other words, subcircuits can be connected to one another to form a snake shape. This type of assembly 600 may utilize at least two types of subcircuits, including some subcircuits 102 that connect left or right and some larger subcircuits 602a, 602b (collectively referred to as 602) that are larger than subcircuits 102. If the area of subcircuit 102 is taken as a single unit of measurement, larger subcircuits 602 may have an area equal to two units, three units (e.g., subcircuit 602a), four units, five units, six units, seven units (e.g., subcircuit 602b), and so on. In some embodiments, the larger subcircuits 602 have one or more dimensions that are 1.1 times, 1.2 times, 1.3 times, etc. the corresponding dimension of subcircuit 102. This type of assembly 600 can be beneficial when numerous subcircuits need to be cascaded, the footprint needs to be reduced, and/or occasional connections (e.g., via photonic wirebonds) need to be made. For example, cascading the subcircuits may be advantageous in some implementations and can include connecting one subcircuit to another in loops (instead of one long linear assembly) to reduce the overall footprint of the integrated photonics assembly. The empty spaces 502 between subcircuits 102 allow for ease of electrical wirebonding 112 to the underlying PCB 406.

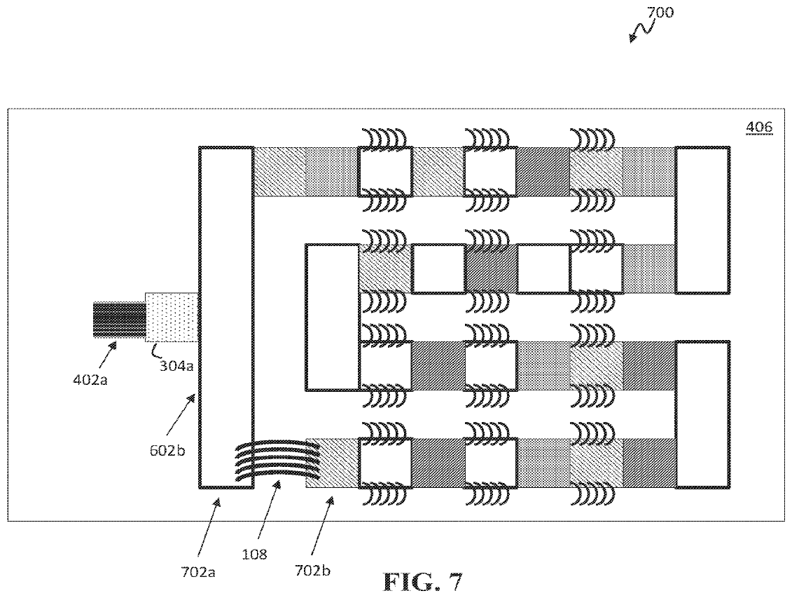

FIG. 7 shows an example of a packaged integrated photonics assembly 700 which is formed in the shape of an open-loop “snake”. This type of assembly 700 can be useful when subcircuits vary slightly in size, leading to a mismatch in size in at least one portion of the assembly 700. This can occur, for example, when the subcircuits are diced during fabrication. Accordingly, a subcircuit connection (e.g., the last connection) can be performed using one or more photonic wirebonds 108 to connect the light paths between subcircuit 702a and subcircuit 702b. This can be used instead of coupling techniques, e.g., butt-coupling.

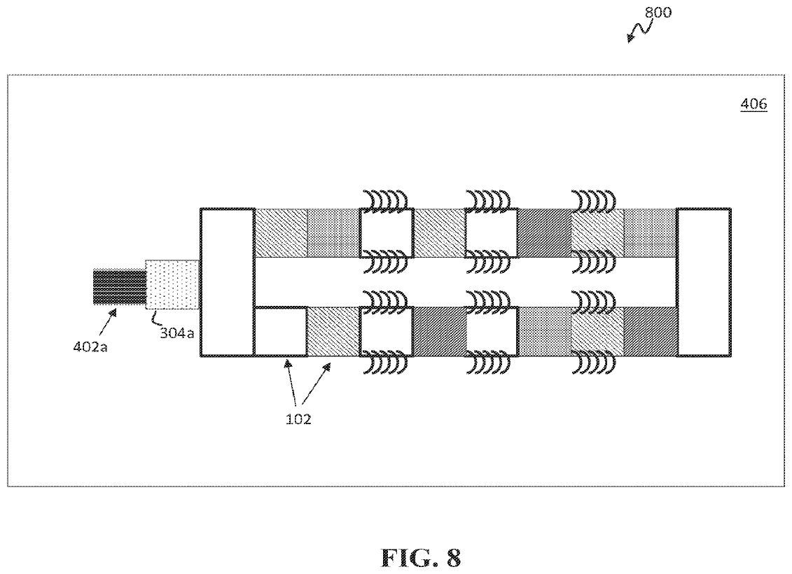

FIG. 8 shows an example of a packaged assembly 800, illustrating that the subcircuits can be standardized. In other words, the subcircuits 102 can be cut to a standard size (within a particular tolerance) such that they can form a closed loop when butt-coupled. For example, during dicing of the subcircuits during fabrication, a given dimension (e.g., width, length, height, etc.) of the subcircuits may vary +/−10 microns. In some embodiments, the resulting variation depends on the particular fabrication process or type of subcircuit produced.

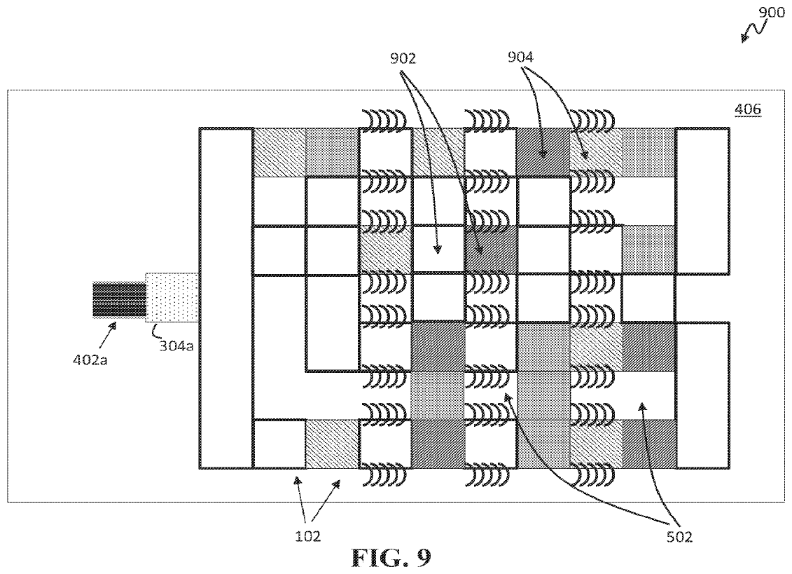

FIG. 9 shows an example of an integrated photonics assembly 900 that is formed into a “checker” type assembly. The checker-type assembly includes empty spaces or gaps 502 between subcircuits 102. These gaps 502 can permit the wirebonding of some or all subcircuits 102 to the host PCB 406 without needing to route electrical signals from the subcircuits (e.g., subcircuit 902) near the center of the assembly 900 to the outer subcircuits (e.g., subcircuits 904) and/or to external circuits.

### Light Transfer In Photonic Integrated Subcircuits

FIG. 10A depicts an example implementation of a 1D integrated photonics assembly 1000. Referring to the subcircuits from left to right, the example assembly 1000 includes:
(i) a subcircuit 1002a including a fiber spot-size convertors;
(ii) a subcircuit 1004a including tunable splitters;
(iii) a subcircuit 1006a including a waveguide crossing;
(iv) a subcircuit 1004b including tunable splitters;
(v) a subcircuit 1006b including a waveguide crossing;
(vi) a subcircuit 1004c including tunable splitters; and
(vii) a subcircuit 1008 including tap couplers and photodetectors 1009 configured to monitor the transmitted light. Subcircuit 1002a can be made from silicon nitride. Subcircuits 1002a, 1002b having fiber spot-size convertors can be made in a different platform which supports higher coupling efficiency to optical fibers. Subcircuit 102a may require a different oxide thickness in the interface 1001a (with fiber array 402a) than the oxide thickness in interface 1001b (with subcircuit 1004a) to efficiently couple light from the fiber array to subcircuit 1004a. Subcircuit 1004a (also referred to as subassembly 1010) can function as a 2×2 optical switch (in this case, including two 2×2 optical switches). Subassembly 1012 of assembly 1000 can function as a 4×4 optical switch. Portion 1014 of assembly 1000 can function as a non-blocking optical switch (e.g., a 4×4 non-blocking optical switch). Subcircuit 1008 can be used enable software control of the optical switch 1014.

Referring to FIGS. 10B-10C, in this example assembly 1000, a subset of the subcircuits is standardized such that these subcircuits (also referred to as standardized subcircuits 1016) have a standard width 1018a (e.g., 1 mm, 1.5 mm, 2 mm, etc.) and a standard length 1018b (e.g., 1 mm, 1.5 mm, 2 mm, etc.). For example, the standardized subcircuits 1016 includes subcircuits 1004a, 1006a, 1004b, 1006b, 1004c, and 1008. The standard subcircuit 1016 has optical and electrical ports are standardized to be in the same respective position for each standardized subcircuit 1016. For instance, in a given standardized subcircuit 1016, input ports 1020 are in the same position along one edge (e.g., the left edge) and output ports 1022 are in the same position along another edge (e.g., the right edge). In some cases, the standardized subcircuit can include electrical ports (e.g., pads) 1024 in the same positions along at least one edge (e.g., top and bottom edges), as indicated by the dashed-line box.

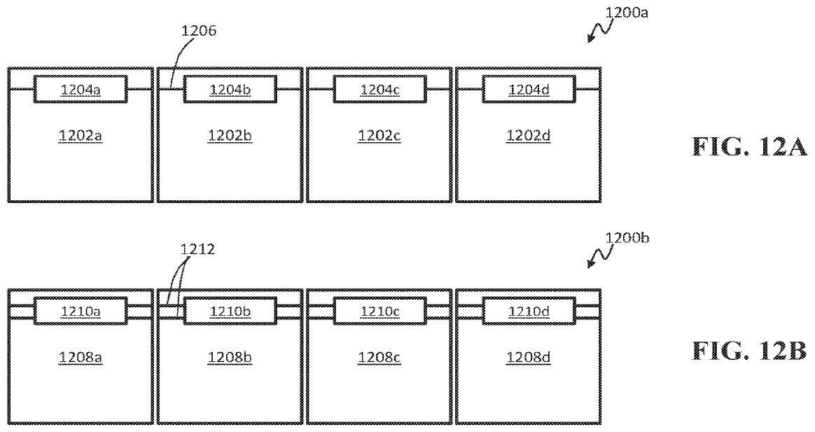

As previously discussed, a subcircuit can be swapped with another subcircuit in a given assembly. Accordingly, FIGS. 11A-11B provide alternative embodiments of the assembly 1000. FIG. 11A illustrates assembly 1100a in which subcircuit 1008 is swapped for subcircuit 1026. In effect, the monitor photodetectors (of subcircuit 1008) are interchanged for variable optical attenuators 1028 (of subcircuit 1026), thereby generating an assembly 1100a with a different functionality from assembly 1000.

In another example, FIG. 11B illustrates assembly 1100b in which subcircuits 1006a is swapped with subcircuit 1004b. This may be done to alter the functionality of the assembly. Alternatively, in example assembly 1100b, crossing-type subcircuit 1006a is swapped for a tunable splitter-type subcircuit and tunable splitter-type subcircuit 1004b is swapped for a crossing-type subcircuit, relative to the assembly 1100a. This may be helpful when subcircuit 1006a or 1004b is needs to be replaced (e.g., because it is faulty).

### Assembly Monitoring

Described herein are systems, devices, and methods monitoring the integrated photonics assemblies. In sonic implementations, monitoring can include testing the subcircuits and/or using the subcircuits as disposable components in a sensor or other circuit. The monitoring of the assembly may be performed during assembly or post-assembly. The monitoring may be performed one or more times, periodically, intermittently, or continuously.

It can be beneficial to monitor the subcircuits to ensure alignment between two or more subcircuits. The alignment between two or more subcircuits can influence the optical coupling efficiency between the subcircuits. Alignment may be performed using passively and/or actively. In active alignment, a feedback signal may be used to determine whether the subcircuits are aligned. In various embodiments, a monitoring circuit can be configured to be attached to and/or be part of a subcircuit. The monitoring circuit may monitor light that couples into the subcircuit. A light path can be configured such that at least a portion of the received light can travel through the monitoring circuit. The light may then be transmitted back out of subcircuit.

An example monitoring system (e.g., including the monitoring circuit) can include a laser and a photodetector to determine optical loss within a subcircuit and/or among subcircuits. This arrangement may permit measurement of the quality of the optical coupling between the subcircuits. The measurement can be used to determine how well the subcircuits are aligned. In some embodiments, once the subcircuits are aligned and fixed in position (e.g., in an assembly), a monitoring circuit is used to determine the coupling efficiency between the subcircuits at any time.

In various embodiments, two subcircuits can be aligned such that there is less than 1 dB, less than 0.5 dB, less than 0.1 dB, less than 0.5 dB, or less of optical loss in light transfer between the two subcircuits. In various embodiments, two subcircuits can be aligned such that there is greater than 75%, greater than 80%, greater than 85%, greater than 90%, greater than 95%, greater than 97%, greater than 99%, or more coupling efficiency.

Passive alignment techniques can include aligning the subcircuits by visual inspection and/or self-alignment techniques. A monitoring circuit may be used to determine the degree of alignment between subcircuits based on passive alignment.

In some embodiments, the monitoring circuit for each subcircuit is individually configured. The subcircuit may have a wavelength dependence and, based on this wavelength dependence, the monitoring circuit may monitor the response of the respective subcircuit. If, for example, the wavelength dependence of the subcircuit has changed, then the monitoring circuit may isolate the single subcircuit response to align or monitor the subcircuit further. In some embodiments, monitoring circuits include photodetectors to monitor light emitted by the subcircuits through grating couplers. Such a monitoring circuit may benefit from a detection method above the subcircuits, which can be used by a camera configured to detect light from photonic circuits to distinguish between the light emitted from the top of each subcircuit. In some embodiments, the monitoring circuit is configured to access the metal pads of each subcircuit to monitor the response of the respective subcircuit.

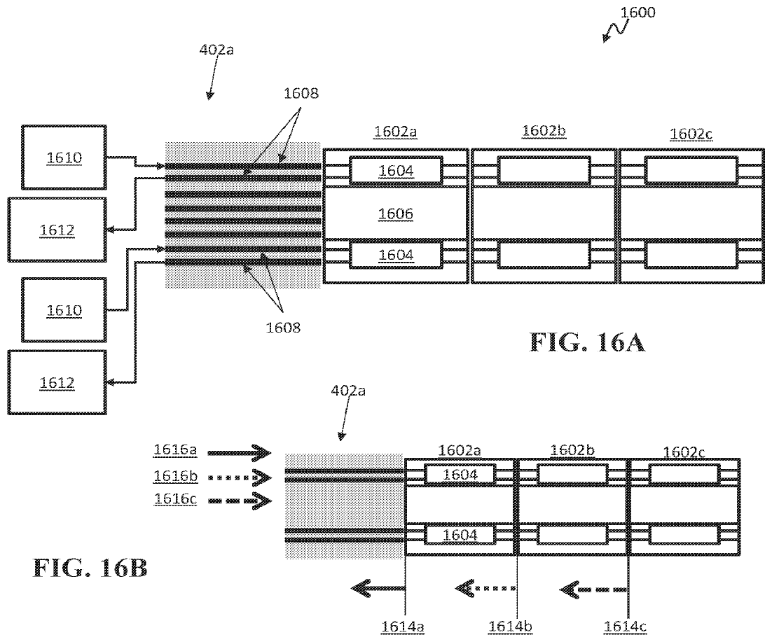

FIG. 12A depicts an assembly 1200a of four integrated photonic subcircuits 1202a, 1202b, 1202c, 1202d (collectively referred to as 1202), in which each subcircuit is configured to transfer light to an adjacent subcircuit. Each subcircuit 1202 is coupled to and/or includes a respective monitoring circuit 1204 (including circuits 1204a, 1204b, 1204c, 1204d). The monitoring circuit 1204 can determine (e.g., measure) the optical coupling efficiency between two subcircuits by monitoring the light traveling in the light path 1206. In some embodiments, these circuits are configured such that the coupling efficiency of a particular subcircuit-to-subcircuit interface may be distinguished from one another (refer to FIGS. 16A-16B for an illustrative example). The monitoring circuit may enable subcircuits to be added or optically coupled to a base subcircuit one-by-one while maintaining a high coupling efficiency. In some cases, each monitoring circuit may include multiple input and/or output waveguides. For example, with two waveguides, light may be transmitted and received. This may reduce the need for additional external components and, in some cases, reduce ambiguity of where the light originates. Additionally or alternatively, a single light path or more than two light paths may be used. FIG. 12B shows four integrated photonic subcircuits 1208a, 1208b, 1208c, 1208d (collectively referred to as 1208), in which each subcircuit is configured to transfer light to an adjacent subcircuit. Each subcircuit 1208 is coupled to and/or includes a respective monitoring circuit 1210 (including circuits 1210a, 1210b, 1210c, 1210d). In this case, two light transfer paths 1212 are used between each subcircuit to determine respective optical coupling. The two light paths may be useful for determining optical coupling efficiency in both directions (e.g., the two opposite directions). In some embodiments, more than two light paths are monitored.

To enable facile alignment monitoring, two monitoring circuits may be placed on opposite sides of the subcircuits. This may increase angular alignment accuracy. For example, this double optical coupling monitoring may increase the rotation alignment accuracy for various optical coupling methods. As described below, in the space on a subcircuit between the two monitoring circuits (;e.g., 1304a and 1304b), a “useful” circuit may be placed such that the monitoring circuits and useful circuit do not interfere with each other. The useful circuit may have independent functionality and/or purpose. For example, by bringing together the useful circuits may the function of the overall assembly be realized.

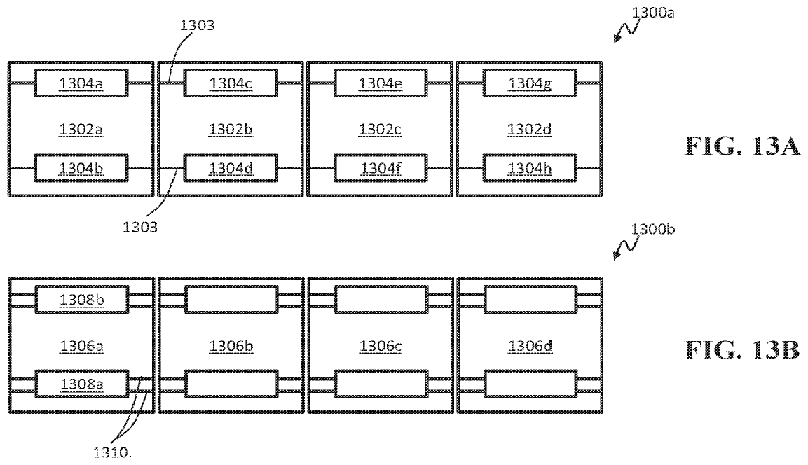

FIG. 13A shows four integrated photonic subcircuits 1302a, 1302b 1302c, 1302d (collectively referred to as 1302), in which each subcircuit is configured to transfer light to an adjacent subcircuit. In this case, for each subcircuit 1302, two respective monitoring circuits 1304 with light paths 1303 are used between each subcircuit to determine optical coupling. Therefore, subcircuit 1302a has monitoring circuits 1304a, 1304b; subcircuit 1302b has monitoring circuits 1304c, 1304d; subcircuits 1302c has monitoring circuits 1304e, 1304f; and subcircuit 1304g, 1304h. The two monitoring circuits may be positioned on opposite sides of a subcircuit, FIG. 13B illustrates a similar set of photonic subcircuits 1306a, 1306b 1306c, 1306d (collectively referred to as 1306), in which each subcircuit has two respective monitoring circuits. For example, subcircuit 1306a has monitoring circuits 1308a, 1308b and each monitoring circuit monitors a respective two light paths 1310.

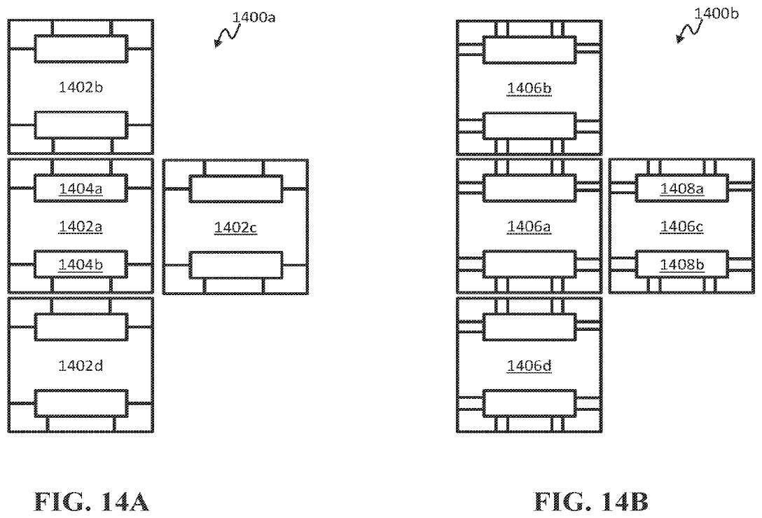

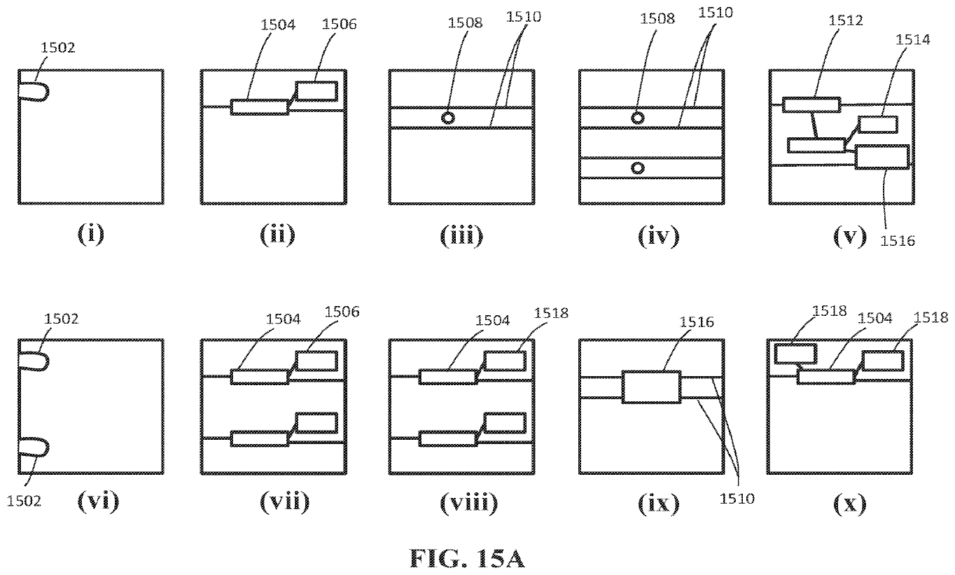

FIGS. 14A-14B illustrate example assemblies 1400a, 1400b of four subcircuits each. In this case, the subcircuits are assembled in two dimensions. For example, assembly 1400a includes subcircuit 1402a coupled to each of subcircuit 1402b, 1402c, and 1402d. Each subcircuit has a monitoring circuit configured to monitor light in two dimensions. For example, subcircuit 1402a has a first monitoring circuit 1404a and a second monitoring circuit 1404b. Assembly 1400b includes subcircuits 1406a, 1406b, 1406c, 1406d in which each subcircuit includes two respective monitoring circuits (e.g., 1408a, 1408b), in which each monitoring circuit has two respective light paths.

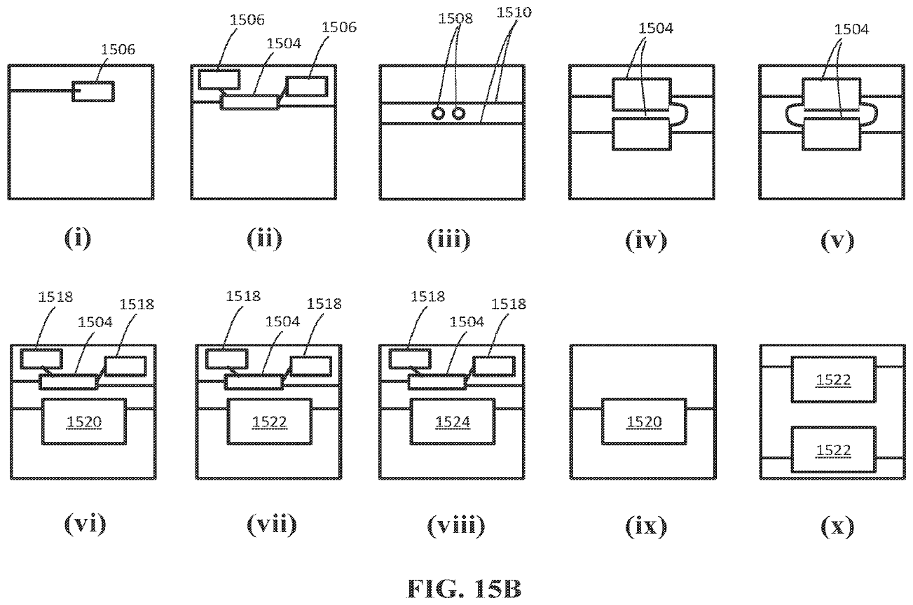

FIGS. 15A-15B provide several examples of photonic monitoring circuit implementations, e.g., configured to determine whether two or more subcircuits are aligned. FIG. 15A(i) depicts a waveguide loopback 1502. This waveguide 1502 receives and returns the light. A photodetector coupled directly or indirectly to this type of subcircuit can determine the degree of alignment (with an adjacent subcircuit) based on the determined optical loss in the returned light. FIG. 15A(ii) depicts a splitter 1504 coupled to a monitoring photodetector 1506. The splitter 1504 is configured to split the received light and send to the photodetector 1506 to determine how well light was received from an adjacent subcircuit.

FIG. 15A(iii) depicts an add-drop ring resonator 1508 which is positioned between two waveguides 1510 and configured to resonate based on the light wavelength in the waveguides 1510. The resonator 1508 may return one or more particular wavelengths. For example, if a given subcircuit has a slightly different add-drop ring, then the monitoring circuit may distinguish the coupling efficiencies for each subcircuit interface. FIG. 15A(iv) depicts a circuit similar to (iii) but with two ring resonators 1508 (each between two waveguides 1510), one situated towards the top and one towards the bottom of the subcircuit. FIG. 15A(v) depicts a double power splitter 1512, followed by a module 1514. The module 1514 can be a photodetector (PD) or out-of-plane coupler (e.g., a grating coupler (GC). The lower splitter (of the double power splitter 1512) can be coupled to a wavelength demultiplexer (WDM)) 1516.

FIG. 15A(vi) depicts a double waveguide loopback 1502. FIG. 15A(vii) depicts two replicas of the circuits of FIG. 15A(ii). FIG. 15A(viii) is similar to FIG. 15A(vii) but employs grating couplers 1518 instead of photodetectors 1506. In this circuit, the out-of-plane emitted light may be detected using a free-space photodetector, a lens system, or a fiber. FIG. 15A(ix) depicts a WDM 1516 coupled between two waveguides. Examples of such circuits include a ring resonator, a WDM having a flat top to make it temperature independent, or a contra-directional coupled Bragg grating reflector. The WDM can be configured to reflect back light at a particular wavelength. FIG. 15A(x) depicts a power splitter with grating couplers 1518 on both sides. Note that most if not all circuits in FIGS. 15A-15B may be configured to monitor light transferred from any side of the subcircuit.

FIG. 15B(i) depicts a waveguide ending in a photodetector 1506. FIG. 15B(ii) depicts a power splitter 1504 with photodetectors 1506. FIG. 15B(iii) depicts a double ring resonator 1508 (e.g., having a flat top), both between waveguides 1510. FIG. 15B(iv) depicts a power splitter 1504 followed by another power splitter 1504. FIG. 15B(v) depicts a circuit similar to FIG. 15B(iv) but useful in both directions. FIG. 15B(vi) depicts a circuit similar to FIG. 15A(viii) but including a separate channel with a wavelength dependent reflector 1520. This can helpful for subcircuits having wavelength-dependent properties, as described herein. FIG. 15B(vii) is similar to circuit of FIG. 15B(vi) but includes a Bragg reflector 1522. FIG. 15B(viii) is similar to the circuit of FIG. 15B(vi) but with a unique wavelength reflector 1524 configured for the particular subcircuit. FIG. 15B(ix) depicts a wavelength dependent reflector 1520 as the sole alignment circuit. FIG. 15B(x) depicts two Bragg reflectors 1522 to measure the alignment accuracy at two different points using the reflected light.

In the above-described monitoring circuits of FIGS. 15A-15B, the following features may be included. The splitters may have any splitting ratio or implementation. The grating couplers (GC) may emit light out-of-plane, configured at a specific angle. The monitoring circuit may utilize one or more GCs that emit at different angles to distinguish between subcircuits. The wavelength demultiplexer (WDM) may have any implementation including, e.g., ring resonators, allelic gratings, Bragg gratings, arrayed waveguide gratings, counter-directional coupling Bragg gratings, etc. In some embodiments, the WDM is configured such that its response is temperature independent, i.e., a flat-top response over a certain wavelength band. This can help ensure that the alignment accuracy monitoring does not change as a function of temperature but only as a function of misalignment. This may be important when monitoring the alignment accuracy during fabrication (e.g., during UV or thermal curing epoxy).

FIG. 16A shows an example one-dimensional assembly 1600 of three subcircuits 1602a, 1602b, 1602c (collectively 1602), in which each subcircuit has at least one monitoring circuit 1604 and a useful circuit 1606. Together, the useful circuits 1606 of two or more subcircuits 1602 may form a larger useful circuit. One of the subcircuits 1602 may be coupled to a fiber array 402a. The fiber array 402a can include multiple optical fiber. The outer fibers 1608 of the fiber array 402a may be used for monitoring the optical coupling efficiency between subcircuits 1602. A laser 1610 may be coupled to the fiber array to provide the light source, e.g., for monitoring the alignment between subcircuits. In some cases, a photodetector 1612 can be coupled into the fiber array and used to monitor light externally.

As discussed above, the subcircuits and/or their respective interfaces can be configured to be wavelength dependent. FIG. 16B provides a simplified representation of FIG. 16A to illustrate an example of wavelength dependence of the interfaces 1614a, 1614b, 1614c between the subcircuits. In this example, interface 1614a is between fiber array 402 and subcircuit 1602a and responds to light with wavelength 1616a; interface 1614b is between subcircuit 1602a and subcircuit 1602b and responds to light with wavelength 1616b, interface 1614c is between subcircuit 1602b and subcircuit 1602c and responds to light with wavelength 1616c. Based on the response from an interface, the photodetector(s) 1612 coupled to the front of the fiber array 402a is able to determine how well aligned two adjacent subcircuits are. In some embodiments, the laser 1610 is tunable to tune the wavelength of the inputted light to the specific WDM of the particular subcircuit (e.g., subcircuit 1602b and not 1602a) in the assembly 1600. In this example, each monitoring circuit can include a WDM to enable the PD 1612 to determine the coupling efficiency between subcircuit 1602a and 1602b, not between 1602b and 1602c.

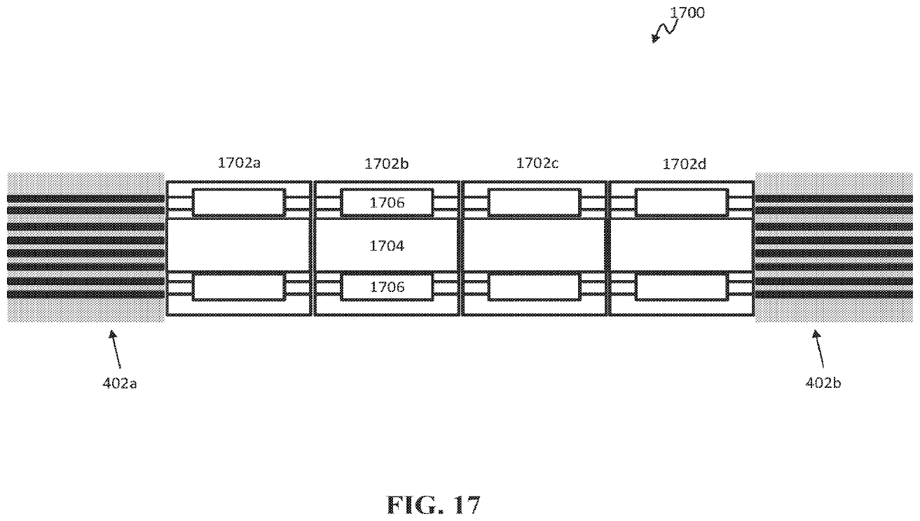

FIG. 17 illustrates a one-dimensional assembly 1700 of four subcircuits 1702a, 1702b, 1702c, 1702d (collectively referred to as 1702). In this case, light can be coupled to fibers on the left and/or right through a first fiber array 402a and a second fiber array 402b. It can be beneficial for monitoring circuits to monitor light bi-directionally. In particular, there are many ways to build this assembly 1700. For example, the assembly 1700 may be initiated from the left by coupling the fiber array 402a to subcircuit 1702a. Alternatively, the assembly may be initiated from the right by coupling fiber array 402b to subcircuit 1702d. In some embodiments, the assembly 1700 is constructed in two or more portions (e.g., partly from the left and partly from the right). In some embodiments, the subcircuits 1702 is assembled before adding the fiber arrays 402a, 402b. As subcircuits 1702 are added to the assembly, the monitor circuits are used as a feedback mechanism to monitor alignment.

### Assembly Alignment and Packaging

Described herein are example systems and methods for passive alignment and/or active alignment of subcircuits. In various embodiments described herein, the alignment systems and methods may feature a receptacle configured with complementary alignment features that can be used to assemble and optically connect many subcircuits at a given time. Further, the subcircuits may be configured to interact with the receptacles to achieve alignment.

As previously discussed, for some subcircuits, the transfer of light is in-plane and by butt-coupling the facet of one subcircuit is positioned adjacent to the facet of the other subcircuit. The input and output optical modes of the subcircuits are configured such that the output(s) match as closely as possible to the input(s) in order to enhance the coupling efficiency. In some embodiments, the mode at the output of the first subcircuit is configured to match the mode at the input of the second subcircuit, adjacent to the first. The modes may be configured even if the waveguide output and input cross-sections themselves are different sizes.

The mode can be configured to be significantly large in order to increase the alignment tolerance of the subcircuits with respect to each other. For example, a mode size can be 3 um, which translates into a 300 nm alignment accuracy for 0.2 dB insertion loss. One way to create such a large mode is to use an optical spot-size convertor on the subcircuits which adiabatically converts a small optical mode from a waveguide to a large mode at the edge of the subcircuit. For example, an implementation of a spot-size convertor is an inverted taper.

Furthermore, the input/output waveguide may be angled in-plane with respect to the facet of the subcircuit in order to reduce back reflections. Anti-reflection coatings may be applied to the subcircuit facets in order to reduce reflections further. In order to get efficient optical coupling between the subcircuits, it is beneficial for all six axes of the subcircuits to be optimized accurately. For instance, two subcircuits can be aligned in the x, y, z axes and all three angles (pitch, roll, and yaw) such that the optical input and output modes of the subcircuit travel along the same axis and to make sure that the subcircuits may be attached with a minimal gap in between.

One way to align subcircuits in six degrees of freedom is to use a hexapod and actively monitor the coupling efficiency between the subcircuits. This method is very cumbersome and slow because light needs to be coupled in and out of the subcircuits while aligning, or an infrared camera needs to be used, etc. It is also a serial process where one may only align one subcircuit at a time, which is not cost-effective when combining, for example, 10 or 20 subcircuits.

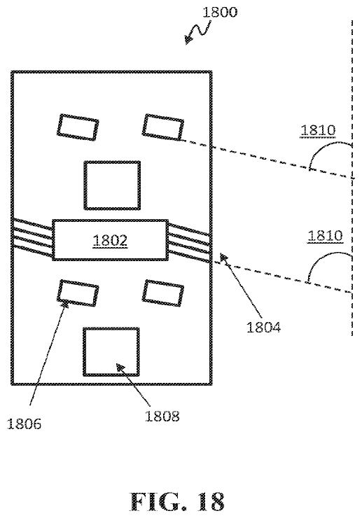

One aspect of the present disclosure is a method to align or pre-align optical subcircuits by passive alignment techniques. The subcircuits can be placed on a receptacle that is fabricated separately. FIG. 18 illustrates the top view of an example embodiment of a subcircuit 1800. The subcircuit 1800 includes a photonic circuit 1802, input and output waveguides 1804, and features 1806, 1808 for passive and/or semi-passive alignment. The deep trench features 1806 may be angled having the same angle 1810 as the waveguides 1804. The oxide open 1808 may be rectangular without a rotation relative to the subcircuit. These alignment features are configured to mate with the complementary features of the appropriate receptacle.

In FIG. 18, the alignment features can be formed by etching in the subcircuit a so-called oxide open 1808, which etches up to the core layer of the waveguide, and a deep trench 1806. which etches to more than 50 um deep. Other alignment features may be used including pyramids, inverted pyramids, v-grooves, features that 3D-printed of any shape, features that are formed using nano-imprint, features that are formed using photo-sensitive resist or polymer (SU8), etc. Each alignment feature is responsible for the passive alignment of at least one degree of freedom. Multiple alignment features may have the same functionality and be redundant or create an elastic averaging effect which increases the alignment accuracy.

The subcircuit can be fabricated on a wafer-scale. The wafer can then be diced to create the subcircuits. An important boundary condition is that the size of the subcircuits may vary since the dicing positional accuracy is typically +/−15 um. In some cases, this boundary condition can be compensated for in the alignment features.

It is beneficial for the edge of the subcircuit where light transfer occurs be in ideal or near ideal condition. The edge may have a side wall angle of 90 degrees. In some embodiments, the edge of the subcircuit has another angle such that two adjacent subcircuits have complementary angles or angles that are negative such that the input and output points of the waveguides may be aligned very close together in order to reduce the diffraction efficiency loss. The subcircuit facet may be smoothed using mechanical polishing or stealth dicing to create a smooth optical facet.

The degree of freedom along the x-direction, i.e., the direction along the width or along the direction of the input/output waveguides as in FIG. 18, is fixed by pushing the two subcircuits against each other until the two subcircuits physically touch. This can be important because the subcircuit dimensions may not be accurately fabricated due to dicing variations. One or more degrees of freedom may be aligned using the alignment features. In the alignment feature implementation of FIG. 18, the vertical alignment (z-axis) is fixed using an oxide etch feature. This etch removes the oxide from the top of the waveguide. The height reference is then the top of the waveguide which is close to the middle of the mode-size. The height reference may be anywhere in the subcircuit stack as long as it results in height matching of mode-sizes of the adjacent subcircuits. Not all subcircuits may be fabricated in the same process and have the same stack-up, so the height reference etch may be different.

When the subcircuits have the exact same distance from the core waveguide layer to the top of the subcircuit, then the top of the subcircuit may be used as height reference. However, this is may be atypical since even wafer-to-wafer or intra-wafer variations of the top layer may occur. FIG. 18 illustrates two oxide etches for the vertical alignment but typically at least three positions are needed with these height reference features which then constrains the height, tip and tilt at the same time. The y degree of freedom or the degree of freedom perpendicular to the waveguide direction can be fixed using the deep trench etch features. The x direction or waveguide direction is not constrained using alignment features because the chips may be pushed against each other and physically touch. The rotational degree of freedom can be constrained due to the fact that there is more than 1 lateral alignment feature.

In some embodiments, two or three lateral alignment features are used for a given subcircuit but more features may be in order to leverage elastic averaging. This is particularly true when the subcircuit and/or receptacle alignment features are made of a non-rigid material. Rotational alignment may be attained using the pick and place tool by referencing the edges of the subcircuit or by pushing the subcircuit edges to each other thereby constraining the rotation. Note that the deep trench etch in FIG. 18, which acting as a lateral alignment feature, can be rotated with respect to the subcircuit edge. In some embodiments, this angle of rotation is the same as the angle of the input and output waveguides relative to the edge of the subcircuit. The rotated lateral features thus creates a free degree of freedom along the waveguide direction.

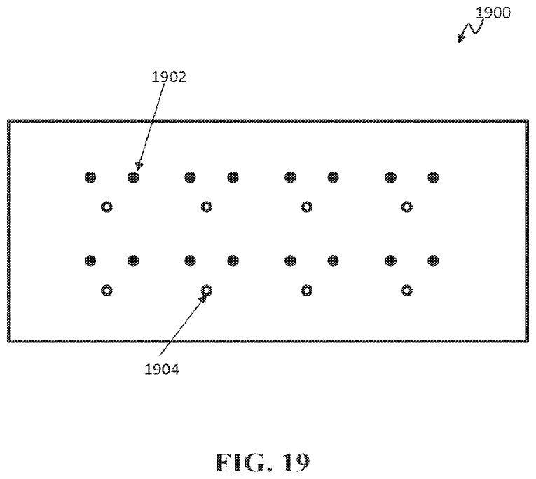

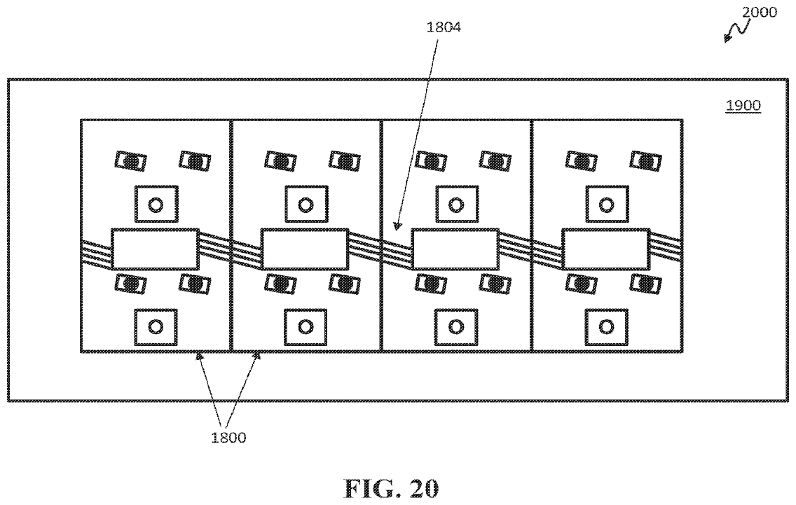

FIG. 19 illustrates the top view of a receptacle 1900 configured to be complementary to the subcircuit 1800. The receptacle includes alignment features with different heights that can mate with the subcircuit. Note that the alignment features can be used to align the subcircuits laterally or vertically. For example, lateral alignment features 1902 can be used to align a subcircuit 1800 laterally relative to the receptacle 1900 and/or to adjacent subcircuits. The lateral alignment features 1902 may be any shape as long as they fit in the deep trench etch hole of the subcircuit 1800 and do not touch the bottom of the deep trench (which would constrain the vertical direction). Vertical alignment features 1904 can be used to align a subcircuit 1800 vertically relative to the receptacle 1900 and/or to adjacent subcircuits. The vertical alignment feature may be any shape as long as it does not touch the edges of the oxide etch of the subcircuit. For example, the lateral or vertical features may have a circular, semi-circular, elliptical, rectangular, or other shape.

FIG. 20 illustrates the top view of multiple subcircuits 1800 positioned on the receptacle 1900. The complementary alignment features of the subcircuits 1802 and receptacle 1900 are configured such that the waveguides 1804 line up perfectly or near perfectly. This can be true even when the width of the subcircuit varies due to dicing.

In some embodiments, between the facets of the subcircuits, an index matching epoxy (e.g., ultraviolet epoxy, thermal epoxy, two-part epoxy, etc.) are added to glue the two subcircuits together. One issue with attaching subcircuits with epoxy is that it takes time to cure the epoxy. Therefore, it may be beneficial if first all or most of the subcircuits are aligned, epoxy is added, and the epoxy between the facets of the subcircuits is cured all at once outside of the pick and place machine. For this, the chips may need to be mechanically held in place in order to not lose alignment. The alignment features contribute to the mechanical stability of the subcircuits relative to the receptacle. However, further reinforcement may be used, e.g., mechanical clamps or vacuum using vacuum holes or lines in the receptacle. After epoxy curing, the epoxy may glue the subcircuits to the receptacle. The subcircuits may be removed from the receptacle by for example treating the receptacle with and anti-adhesive layer before use. The receptacle may then be used multiple times, thereby decreasing assembly cost. One beneficial factor of using a receptacle temporarily and not permanently is that the top of the subcircuit assembly is now accessible and the subcircuits assembly may be packaged (e.g., by wirebonding, fiber array attachment, PCB board mounting, etc.) in a regular fashion with the top side face up.

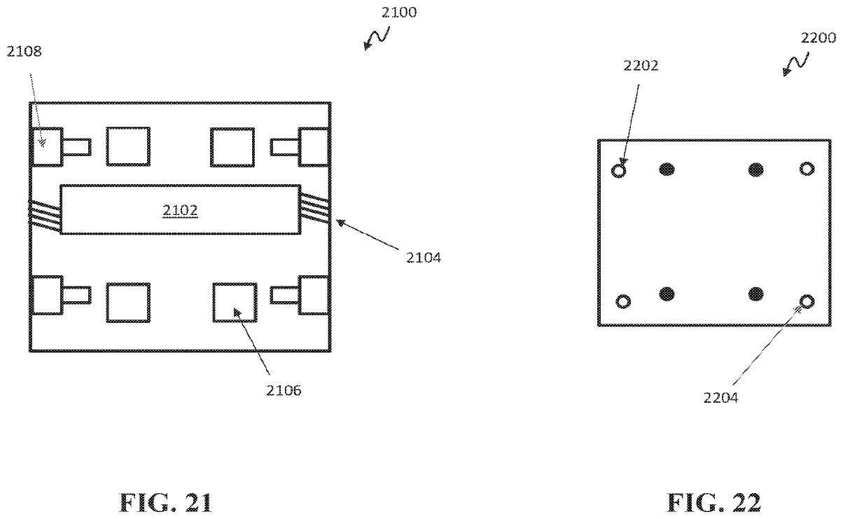

FIG. 21 shows the top view of an example subcircuit 2100 including photonic circuit 2102 and input and output waveguides 2104. The subcircuit 2100 is configured with two types of etches, a shallow-type etch (e.g., oxide open) 2106 and a deep-type etch (e.g., deep trench) 2108. In this case, the waveguides are straight, e.g., the angle of the lateral alignment features is zero. The alignment features may have a specific shape such as a funneling shape to guide the alignment process. In this case, many receptacles are used with each receptacle aligning two subcircuits. This has the benefit of having more versality in terms of chip sizes and process differences. Furthermore, the receptacle does not to be removed since one has access to the top of subcircuits in regions where there is no receptacle.

FIG. 22 illustrates a top view of an example connector chip 2200 that may be used in assembling two subcircuits 2100. The connector chip 2200 can be configured with lateral alignment features 2202 and/or vertical alignment features 2204.

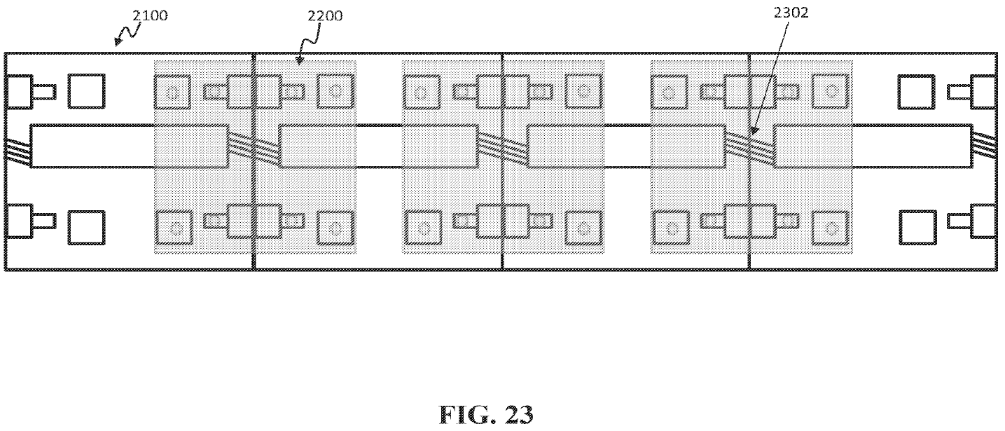

FIG. 23 depicts a top view of an example assembly of subcircuits 2100. The subcircuits 2100 are assembled using connector chips 2200. Each connector chip 2200 combines two subcircuits 2100 such that the input waveguides 2104 of one subcircuit are aligned to the output waveguides 2104 of the other subcircuit (at position 2302).

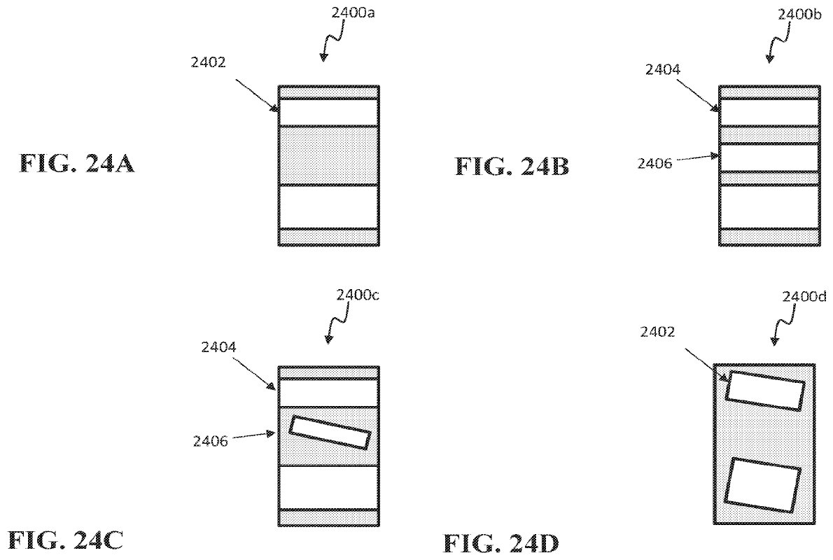

FIGS. 24A-24D shows top views of example variations for subcircuits 2400a, 2400b, 2400c, 2400d (collectively referred to as 2400). The subcircuits 2400 are configured with etches 2402 that may extend or not extend to the edge of the subcircuit. The etches can include oxide open 2404 or a deep trench 2406. The etches 2402 may be non-angled or angled (relative to the subcircuit 2400). The etches 2402 may be used for either vertical or lateral alignment or both.

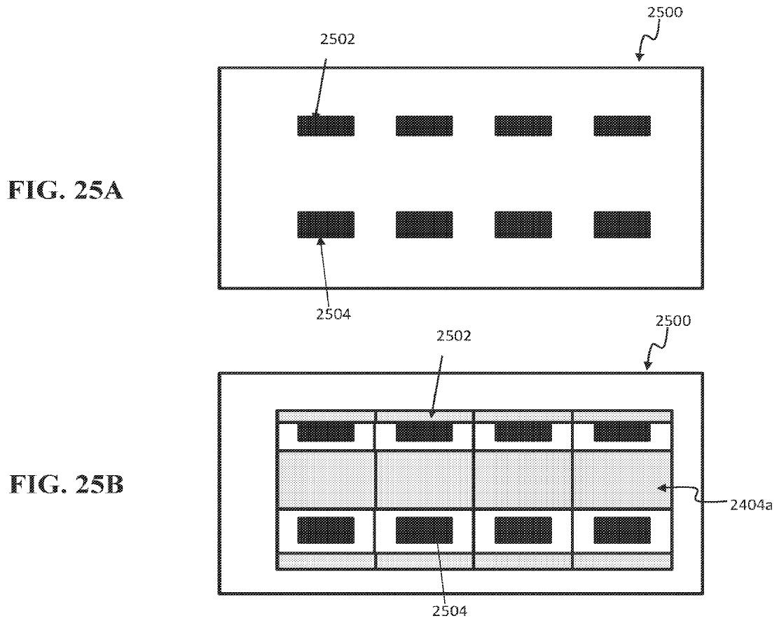

FIG. 25A illustrates a top view of a receptacle 2500 configured to receive subcircuits 2400a, 2400b, and/or 2400c, The receptacle 2500 includes vertical and/or lateral alignment feature 2502 and a vertical alignment feature 2504. FIG. 25B illustrates the top view of the receptacle 2500 connected to four subcircuits 2400a. In this case, the alignment features are more rectangular and the receptacle alignment features are also rectangular, touching with a plane of points instead of a vertical line (compare to FIGS. 18-20). The oxide open etch can be used for both vertical and lateral alignment features. The edge of the northmost alignment feature can be used for lateral and rotational alignment.

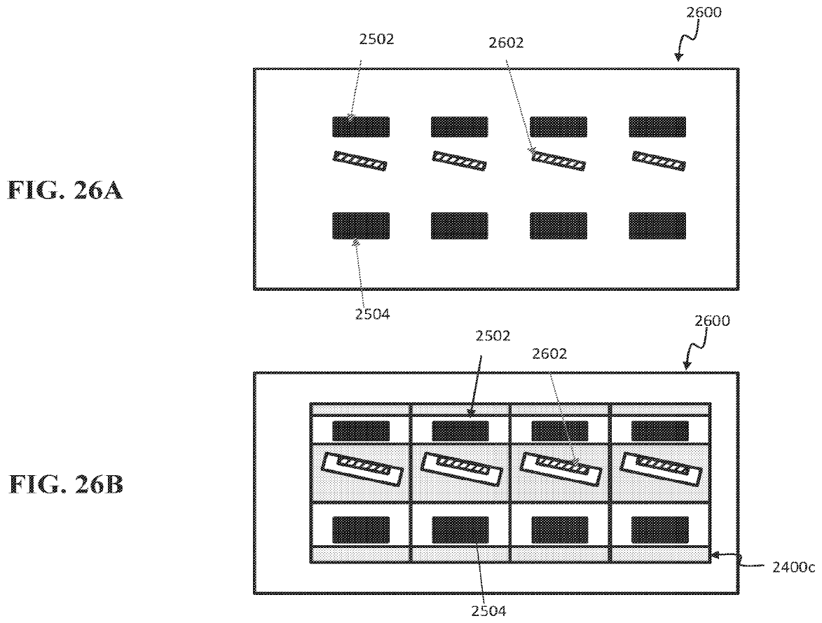

FIG. 26A illustrates a top view of receptacle 2600 configured to receive subcircuit 2400c. In receptacle 2600, the lateral alignment feature 2602 is angled and, once mated with the subcircuit 2400c, only touches one side or edge of subcircuit 2400c. FIG. 26B illustrates the top view of the receptacle 2600 connection to four subcircuits 2400c. The receptacle 2600 features angled lateral alignment features that are rectangular in shape. In this case, the middle alignment features constrain the chip alignment in the lateral and rotational dimensions.

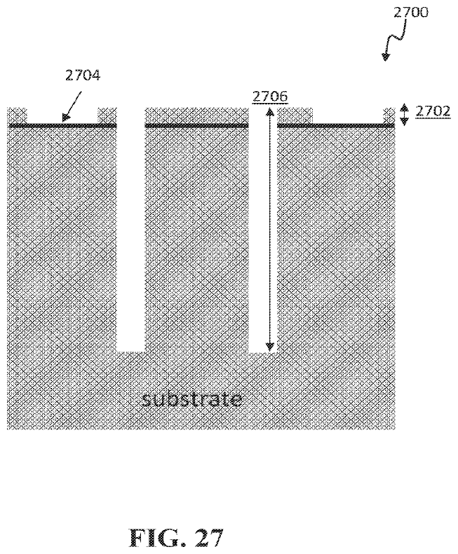

FIG. 27 is a cross-sectional view of an example subcircuit 2700. The subcircuit 2700 has a shallow etch 2702 (e.g., oxide open) of 5 um which stops at or is close to the waveguide layer 2704 and a deep trench etch 2706 of 80 um. The subcircuit 2700 has a waveguide layer 2704 which guides light and may be used to form input and output couplers and photonic circuits. In some embodiments, the generation of alignment features for subcircuit 2700 takes advantage of processes available in every or most of the fabs. Therefore, the deep trench etch and oxide etch can be useful because they are both options that are available in many fabs and may in some cases be fabricated on the same wafer.

The deep trench is typically used for creating a smooth facet for horizontal fiber coupling. Since a standard single mode cleaved fiber has a 125 um diameter, the deep trench is typically more than 62.5 um deep (half of the fiber diameter). As long as the lateral alignment features on the receptacle (blue in FIG. 9) are not taller than 62.5 um they will not touch the bottom of the deep trench and thus not confine the subcircuit in the vertical direction. This is desirable since the depth of a deep trench is typically difficult to accurately control. The oxide open on the other hand may be controlled with nanometer precision. Another benefit of using the deep trench is that the area that is used for lateral alignment is comparatively large and thus pretty robust to mechanical damage and wear and tear.

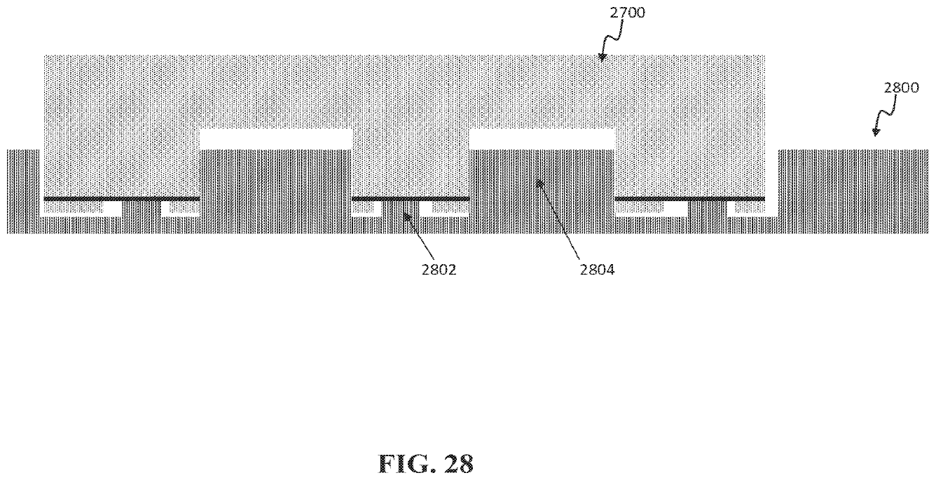

FIG. 28 is a cross-sectional view of the subcircuit 2700 in combination with the receptacle 2800. The example receptable 2800 includes vertical alignment features 2802 configured to mate with the shallow etch 2702 of the subcircuit 2700 and lateral alignment features 2804 configured to make with the deep features 2706.

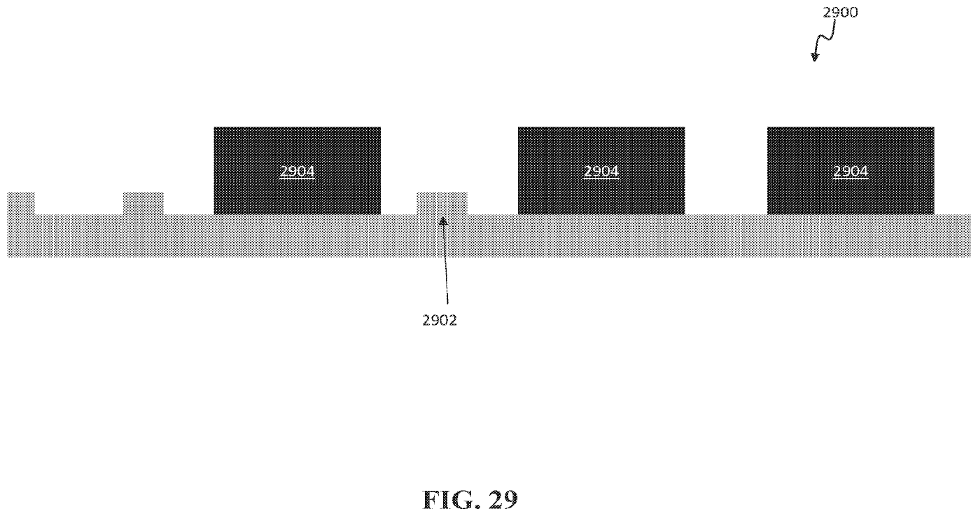

FIG. 29 is a cross-sectional view of an example implementation of a receptacle 2900. The lower profile alignment features 2902 are in glass and are used for vertical alignment. The higher profile alignment features are made of a polymer 2904 and used for lateral alignment.

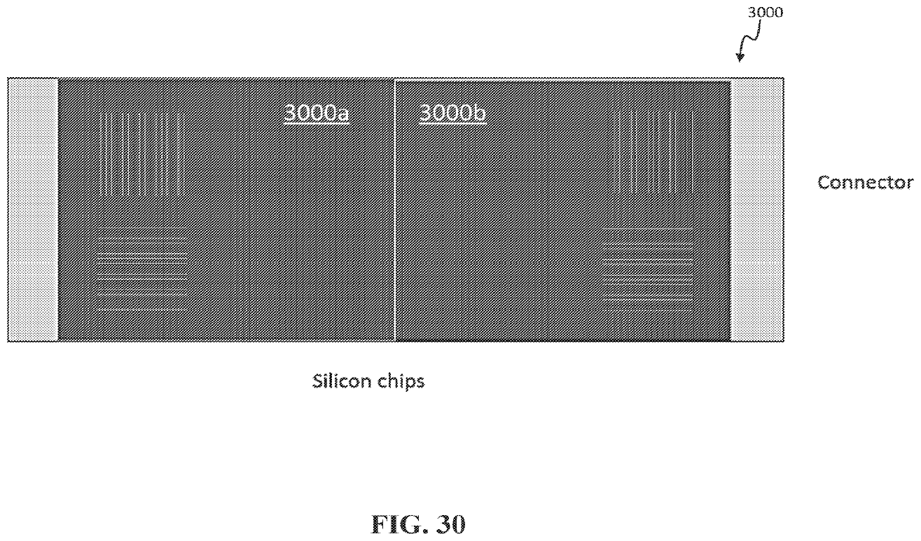

FIG. 30 shows a top view example subcircuit(s) 3000a, 3000b aligned to a receptacle 3002. In this case, the alignment features are designed to be a grating of several slits. Note also that the subcircuit(s) 3100a, 3100b are aligned in mirror-image positions relative to one another, In this case, the alignment features are configured as gratings (e.g., repetitive structure) which may give more freedom to configure elastic averaging for the combination.

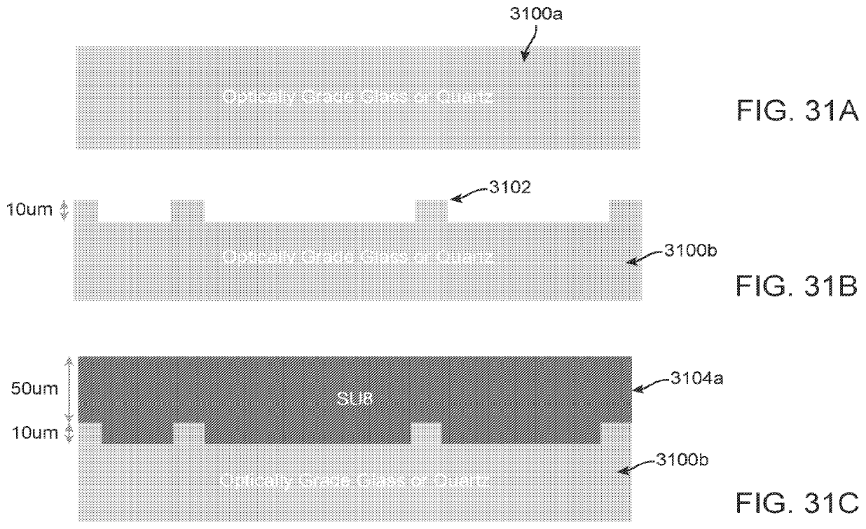

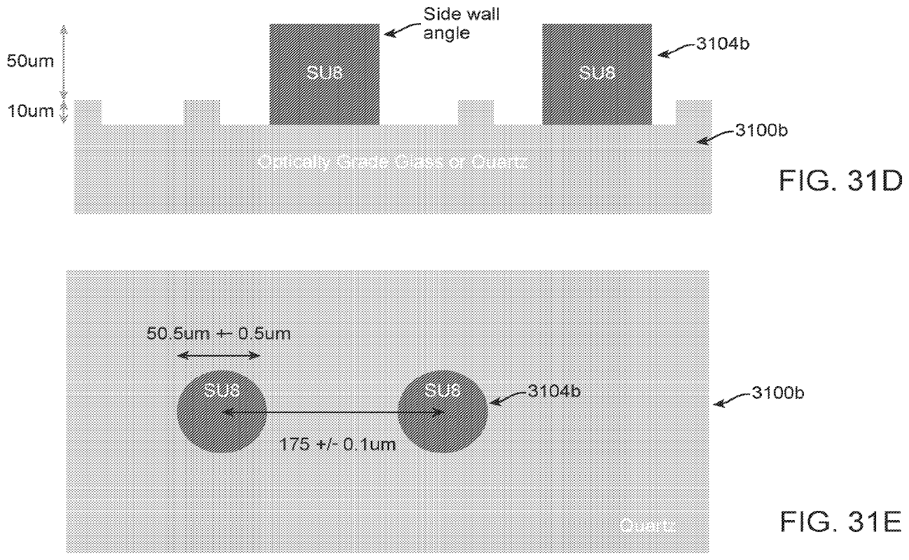

FIGS. 31A-31E illustrate example fabrication steps for fabricating a receptacle wafer. FIGS. 31A-31D provide a cross-sectional view while FIG. 31E provides a top view. Referring to FIG. 31A, optical-grade glass or quartz can be used as a starting substrate 3100a. A flat, transparent substrate can make it easy to visually inspect the alignment. Referring to FIG. 31B, a shallow etch (e.g., of 10 um) is performed to define vertical alignment features 3102 in etched substrate 3100b. The vertical alignment features can be etched (for example, a 10 um etch) with an etch that is deeper than the oxide open etch on the subcircuit (typically ranging from 2 um to 9 um). The top of the glass substrate 3100b now acts as the vertical alignment reference point. This may be beneficial since the glass was mechanically polished to be completely flat (e.g., optically grade flat). In FIG. 31C, the lateral alignment features can be formed in an epoxy or polymer (which is elastic). For instance, a polymer (e.g., SU8) 3104a is spin coated onto the substrate 3100b. In FIG. 31D, the SU8 3104b is patterned to define lateral alignment features. In FIG. 31E, the lateral alignment features are provided in a top view. In this example, each lateral alignment feature is substantially circular with a diameter of 50.5 um+/−0.5 um. These features are separate by 175 um+/−0.1 um.

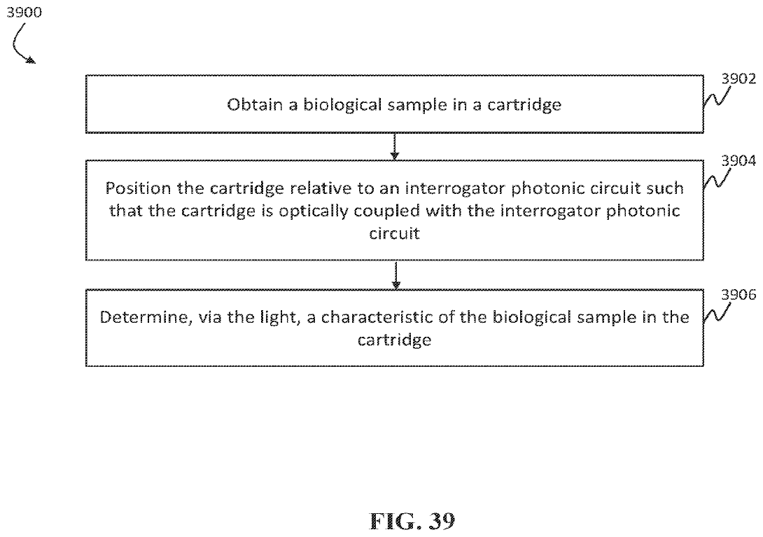

The side wall angle of these features may be configured for easy insert (positive angle) or for better mechanical stability (negative angle). The width of the lateral alignment feature 3104b may be either the same size, a bit narrower or a bit wider than the pit in the subcircuit. Exactly the same size may be ideal but may not be perfectly achieved. If the lateral alignment feature is a bit wider on the receptacle, then it may need to compress a bit to match the trench width in the subcircuit. Another strategy is to make the receptacle features a bit narrower and offset them from the center position. The latter is shown in FIG. 39 in which the left alignment feature touches the right edge of the sidewall of the subcircuit trench and the right alignment feature touches left edge of the sidewall of the subcircuit. More complex elastic averaging strategies may be implemented. In some embodiments, instead of a quartz or glass substrate, a silicon substrate is used. Other materials and substrates may be used for the substrate. In one example, the receptacles may be 3D printed, given the printer has sufficient accuracy.

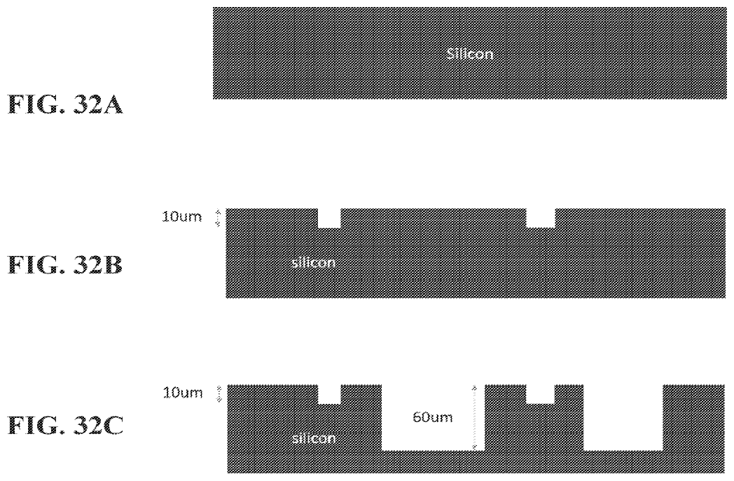

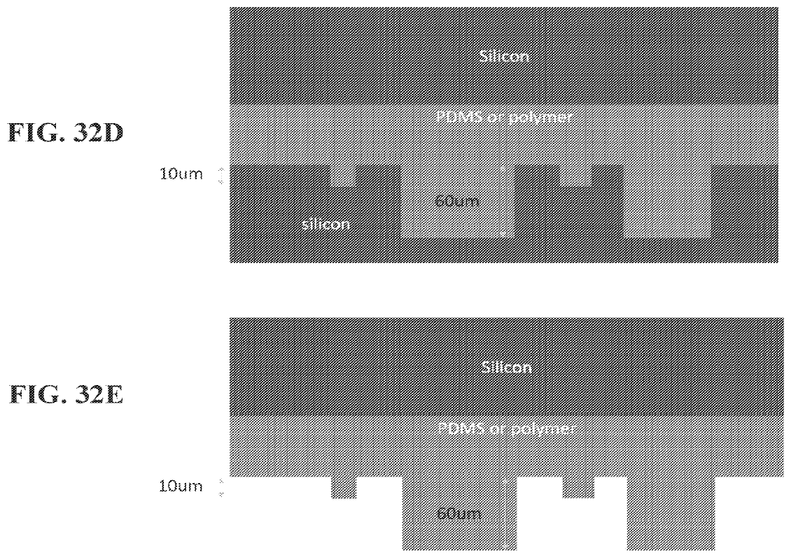

FIGS. 32A-32E illustrate an example alternative method to fabricate a receptacle. FIGS. 32A-32D provide a cross-sectional view while FIG. 32E provides a top view. In FIGS. 32B-32C, the inverse (or mold) is first patterned in a silicon substrate using two etch steps. In FIGS. 32D-32E, using a nanoimprint method, the receptacle is fabricated using the silicon as a mold in PDMS or polymer. One benefit of this approach is that it may reduce the cost of the receptacle itself.

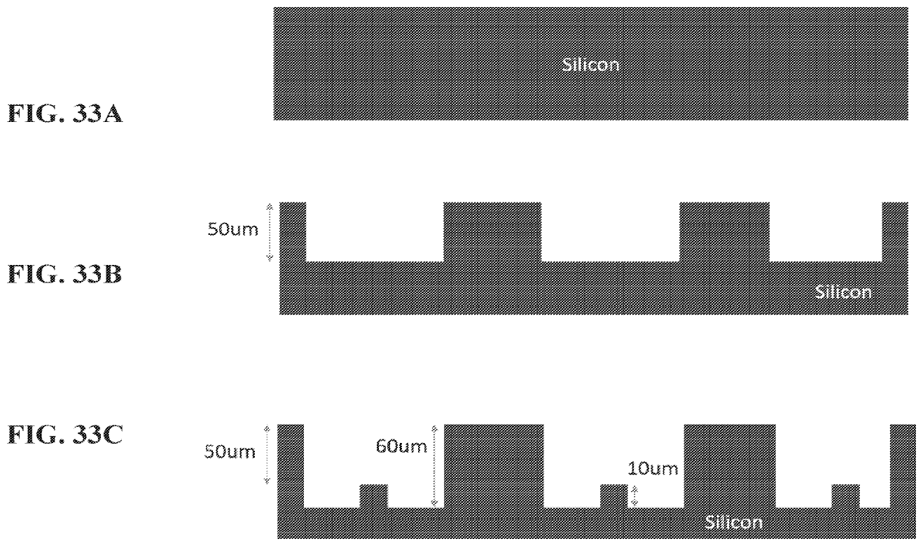

FIGS. 33A-33C illustrate an example method to fabricate the receptacle directly on a silicon wafer. The first etch is then the deepest etch and a second etch is performed to define the vertical alignment features. In FIG. 33B, a deep etch (e.g., of 50 um) is performed. In FIG. 33C, a shallow etch (e.g., of 10 um) is performed into the deep etched pits.

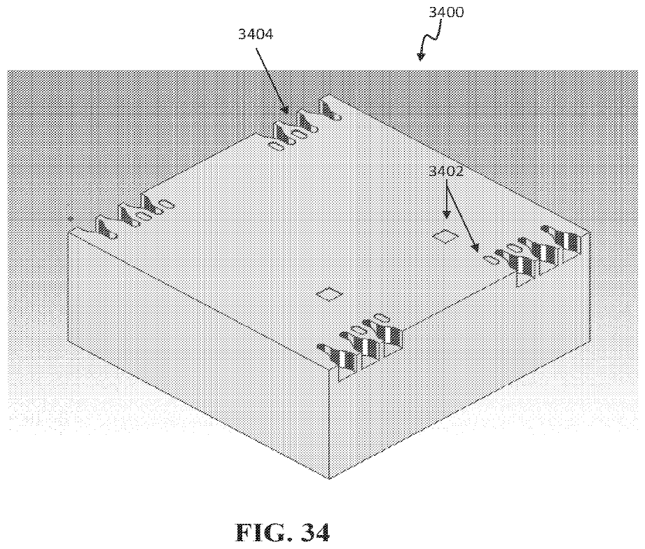

FIG. 34 illustrates an example 3D drawing of a subcircuit 3400 having shallow-etched vertical alignment features 3402 and deep-etched lateral alignment features 3404. The example subcircuit is 2 mm×2 mm with 790 um in thickness. The 3D rendering better illustrates the aspect ratio of the alignment features (deep versus shallow) and the etch depths with respect to the subcircuit thickness.

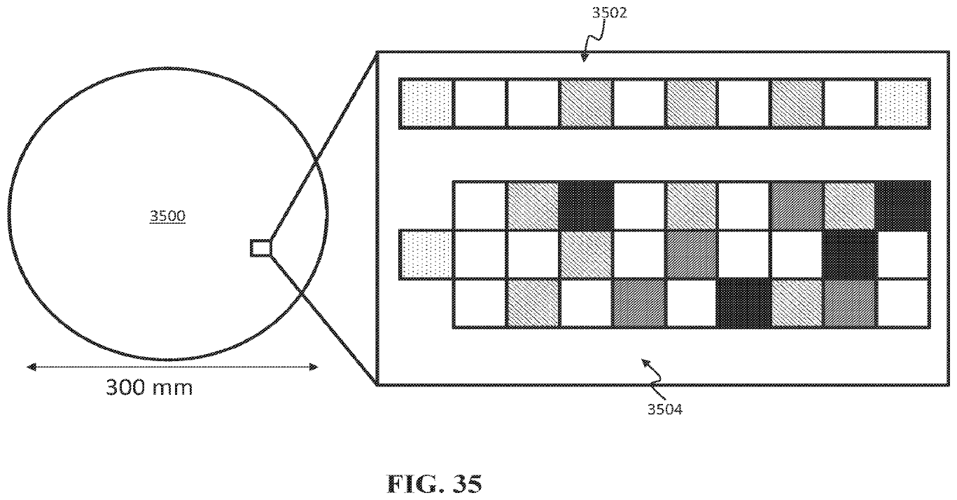

FIG. 35 illustrates the assemblies 3502, 3504 of subcircuits on a receptacle silicon wafer 3500 which may be either 1-dimensional, 2-dimensional, or 1.5-dimensional. The wafer has a approximately 300 mm diameter. The subcircuits have different colors indicating subcircuits from different processes or technologies.

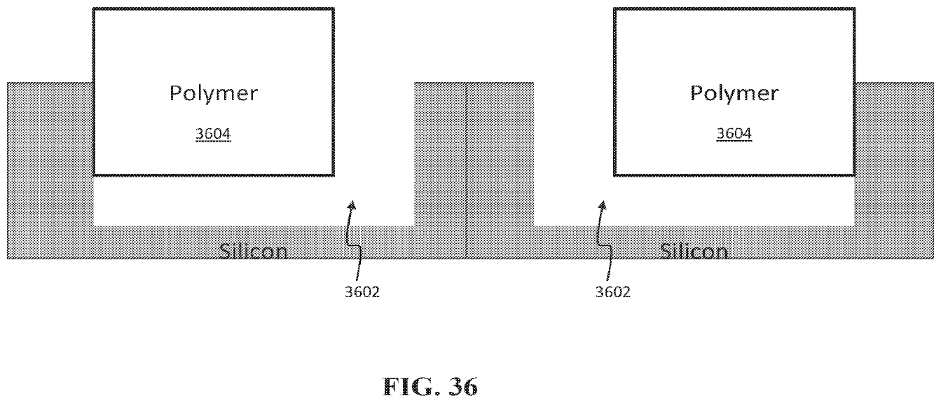

FIG. 36 illustrates a method for aligning two or more subcircuits by using elastic averaging. As described above, subcircuits and receptacles may have lateral alignment features. For instance, in FIG. 36, the receptacle may have cavities 3602 for receiving lateral alignment features 3604 of subcircuits. For example, the use of a polymer for a lateral alignment feature may be beneficial for elastic averaging. By making the lateral alignment features slightly offset, high lateral alignment accuracy may be achieved. In some embodiments, the subcircuits and receptacles each have multiple (e.g., 10 or less, 20 or less, 30 or less, 50 or less, 100 or less) alignment features, which when offset relative to one another, can create accurate positioning and/or connections by averaging the error inherent to the lateral alignment features.

In some embodiments, the coarse alignment is performed passively while the fine final alignment may be performed actively in one or more degrees of freedom, using either optical feedback or vision feedback using alignment marks. One such implementation is to perform a quick final alignment of one of the lateral axes while the height, tip and tilt are passively constrained. The benefit of this is that alignment stage only needs to be able to move in one of the degrees of freedom and does need to be a hexapod type of device.

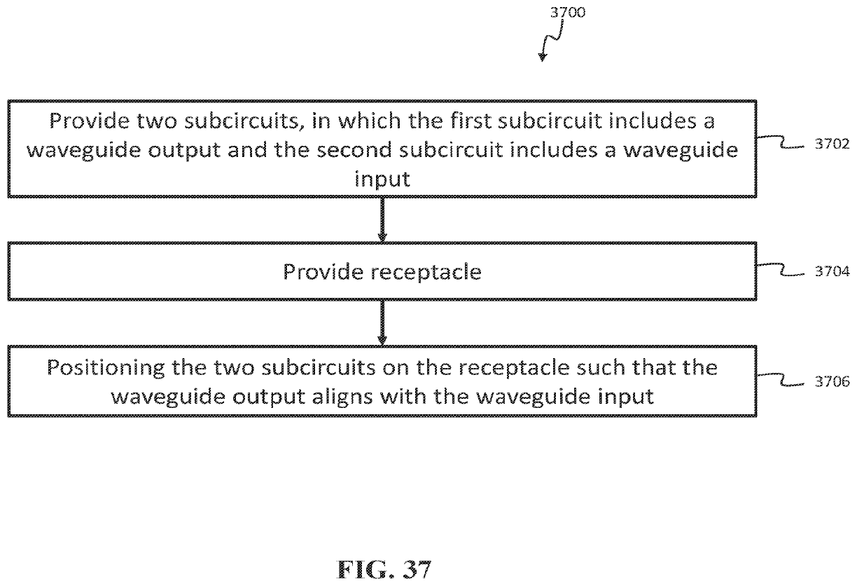

FIG. 37 is a flowchart of an example method 3700 for aligning two or more photonic integrated subcircuits. In step 3702 of method 3700, two or more subcircuits are provided. A first subcircuit may include a waveguide output (e.g., along a first edge) and the second subcircuit can include a waveguide input (e.g., along a second edge). The subcircuit may include at least one subcircuit vertical alignment feature and/or at least one subcircuit lateral alignment feature. In step 3704, at least one receptacle is provided. In some cases, one receptacle is provided for two or more subcircuits. The receptacle may include at least one receptacle vertical alignment feature and/or at least one receptacle lateral alignment feature. The subcircuit vertical alignment feature can be configured to be complementary to the receptacle vertical alignment feature. The subcircuit lateral alignment feature can be configured to be complementary to the receptacle lateral alignment feature. In step 3706, the two subcircuits can be positioned on the receptacle (or the receptacle can be positioned on the two subcircuits) such that the waveguide output of the first subcircuit matches the waveguide input of the second subcircuit. It is understood that the example 3700 method may leverage any embodiment or feature described herein. The subcircuits may be any example embodiment of a subcircuit described herein and/or may include one or more subcircuit features described herein.

### Integrated Photonic Systems and Methods for Biosensing

Described herein are various embodiments of integrated photonic systems and methods for biosensing. In some cases, integrated photonic biosensors can combine high-sensitivity analysis with scalable, low-cost complementary metal-oxide-semiconductor (CMOS) manufacturing. The biosensors may be implemented in portable, highly-accessible, and easy-to-use devices. Example integrated photonic biosensors can include one or more photonic integrated subcircuits, as described above.

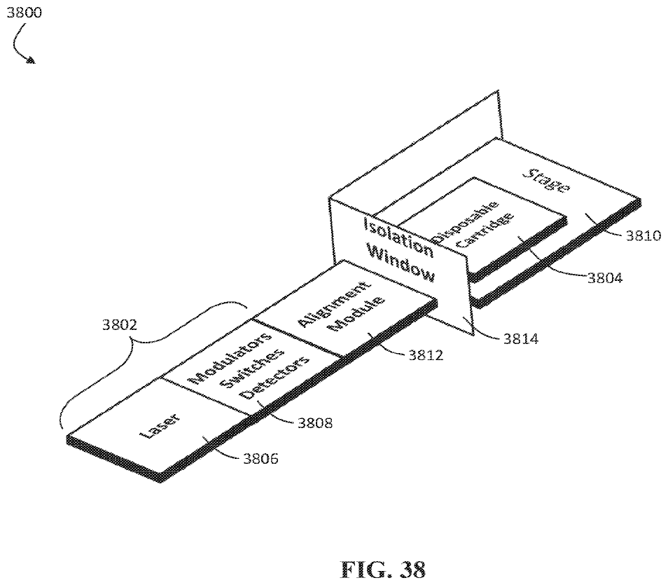

FIG. 38 illustrates an integrated photonic system 3800 for biosensing including an interrogator 3802 and cartridge 3804. The interrogator 3802 may be an assembly including one or more photonic integrated subcircuits, which may each be active or passive. These subcircuits may be packaged together or may be modular. The interrogator 3802 may include a light source 3806 (e.g., a laser) configured to generate a light. A photonic integrated subcircuit may be edge-coupled to the light source 3806 and can include one or more light paths (e.g., waveguides) configured to carry light. The interrogator 3802 can include a control circuit 3808 to control the light in the light paths of the interrogator 3802. In some cases, the interrogator 3802 may be coupled to an interface to provide an electronic and/or visual readout to a user of the system 3800.

The interrogator 3802 can be optically coupled to the cartridge 3804. The cartridge 3804 can be configured to receive a biological sample (e.g., a biological fluid). The light from the interrogator 3802 can be used to determine one or more characteristics of the biological sample in the cartridge 3804. In some embodiments, the cartridge 3804 includes a sensor photonic integrated subcircuit (also referred to as a “sensor subchip”, “sensor chiplet” or simply as “sensor”). In some embodiments, the cartridge 3804 includes a sensor photonic integrated circuit (also referred to as a “sensor PIC” or “sensor assembly”). In some embodiments, the cartridge 3804 includes a microfluidic cell. The microfluidic cell may include one or more proteins (e.g., antigens), one or more reagents, one or more rinsing fluids, etc. The microfluidic cell may include a magnetic microstirrer, a plasmonic vortex mixer, and/or a flow-inducing device. For example, the microfluidic cell may leverage a mixing mechanism or a flow-inducing mechanism to ensure sufficient interaction between the analyte and the sensor chiplet surface. In some embodiments, the microfluidic cell may include a microstirrer and a transmitter (e.g., a magnetic field generator) configured to power the magnetic microstirrer. Note that the cartridge 3804 can be separately packaged (e.g., in a housing) from the other components in the system 3800.

In some embodiments, system 3800 can include a stage 3810 configured to removably engage the cartridge 3804. For instance, the cartridge 3804 may be positioned such that it is temporarily secured (e.g., mechanically) on the stage 3810. The stage 3810 may facilitate alignment (e.g., mechanically) of a light path of the interrogator 3802 and the light path of the cartridge 3804. In some cases, the stage 3810 can include a thermoelectric heater and/or thermoelectric cooler.

FIG. 39 illustrates a method 3900 for biosensing utilizing the integrated photonic system 3800. In step 3902, a biological sample is obtained in the cartridge 3804. In step 3904, to sense a characteristic of the biological sample (e.g., perform a test), a cartridge 3804 is positioned onto the stage 3810. The cartridge 3804 can be positioned such that it is optically coupled with the interrogator 3802. Note that the biological sample may be loaded into the cartridge 3804 before or after the cartridge is placed 3804 onto the stage 3810. In step 3906, the light source 3806 can he activated to determine a characteristic of the biological sample in the cartridge 3804, as described in further detail below.

In some embodiments, system 3800 can include an alignment module 3812 configured to facilitate alignment between a light path of the interrogator 3802 and a light path of the cartridge 3804 (e.g., a light path of the sensor chiplet). The alignment module 3812 may be physically adjacent to the interrogator 3802 or to the cartridge 3804.

The cartridge 3804 may be positioned such that a light path of the cartridge 3804 is aligned with a light path of the interrogator 3802. For example, the cartridge 3804 is aligned to the alignment module 3812 for horizontal optical coupling (e.g., in the plane of the subchip or chiplet). In some embodiments, the alignment may be active, e.g., by monitoring an optical response. In some embodiments, the alignment may be passive using mechanical alignment features of the cartridge 3804, sensor chiplet, and/or stage 3810. After this initial alignment, adjustments may be made to the optics in the alignment module 3812 to increase coupling efficiency. For example, desirable coupling efficiency between the cartridge 3804 and the interrogator 3802 may be at least 10%, at least 20% at least 50%, at least 60%, at least 70%, at least 80%, at least 90%, at least 95%, etc.

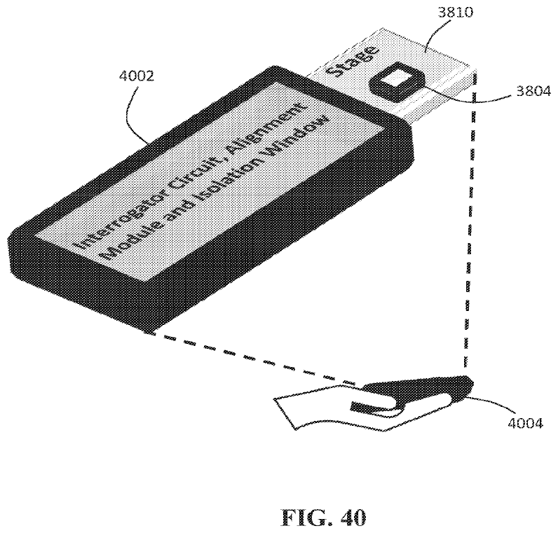

Note that the interrogator 3802 and other components can be reused to receive biological samples via the cartridge 3804. The cartridge 3804 may be disposable after use by a single biological sample. In some embodiments, to prevent contamination, the cartridge 3804 and/or stage 3810 may be physically separated from the alignment module 3812 and interrogator 3802 by a transparent window 3814 (also referred to as an “isolation window”) to ensure no physical cross-contamination. Referring to FIG. 40, in some cases, the interrogator-side components 4002 may be packaged together in a single housing and referred to as a “reader”. The reader 4002 can include the interrogator circuit 3802, alignment module 3812, and/or isolation window 3814. The reader 4002 and the cartridge-side components (e.g., cartridge 3804 and/or 3810) may be configured into a handheld apparatus 4004, as described in further detail below. Due to horizontal optical coupling, compact footprint, and disposability of the cartridge 3804, the arrangement described above enables an inexpensive, hand-held point-of-care device.

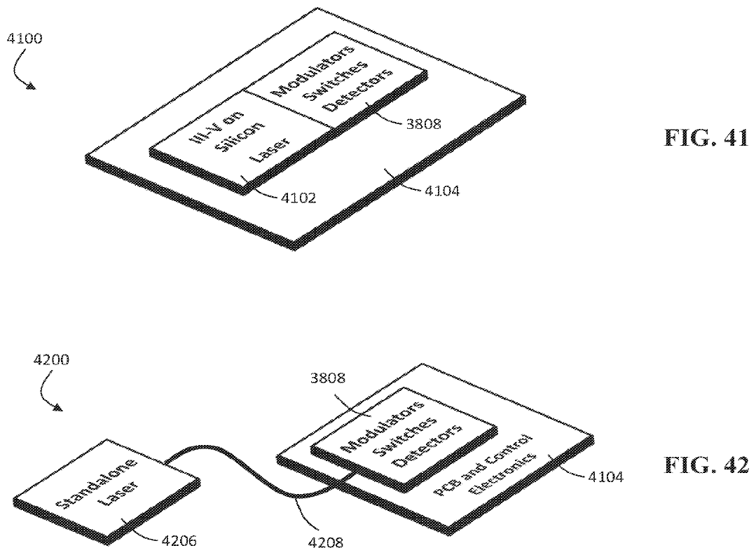

FIGS. 41-42 illustrate embodiments of interrogator 3802. In example interrogator 4100, the light source 4102 (e.g., laser of a III-V on silicon type chip) is edge-coupled to the control circuit 3808. The light source 4102 and control circuit 3808 can be assembled on a PCB 4104.

This configuration may be referred to as “on-chip laser”. In example interrogator 4200, the light source 4206 is a standalone laser connected to the control circuit 3808 by an optical fiber 4208. This configuration may be referred to as “off-chip laser”.

Example alignment modules 3812 may be assemblies that include photonic integrated circuits with edge couplers, grating couplers, micro-electromechanical system (MEMS) mirrors, phased arrays, lenses, and/or fiber arrays. The alignment module 3812 can facilitate the optical coupling between the interrogator 3802 and the sensor chiplet of the cartridge 3804. Once the cartridge 3804 is mechanically aligned on the stage 3810, the interrogator 3802 searches for an optical response from alignment optics on the sensor chiplet (of the cartridge 3804) and/or the cartridge 3804. The interface of the alignment module 3812. can send a signal to determine alignment. The same interface, through the same or different ports, can receive a signal back.

The interface can include an array of edge couplers, a 2D fiber array, a 2D phased array of grating couplers, etc.

In some embodiments, active switches on the alignment module 3812 are tuned to send the signal, e.g., through different output couplers or fibers and/or at a different angle from the phased array. Depending on the captured response, the switches may be tuned to optimize (e.g., increase) coupling. In some examples, in place of a phased array, MEMS mirrors may be employed to beam-steer. The stage 3810 may have active mechanical alignment capability via micro-actuators. The micro-actuators may also be driven using feedback from the alignment module 3802. In order to improve coupling, a ball lens or other lens may be employed to focus the light exchanged between the alignment module 3812 and the sensor chiplet or cartridge 3804. In some cases, this lens may also be moved using micro-actuators to improve optical alignment.

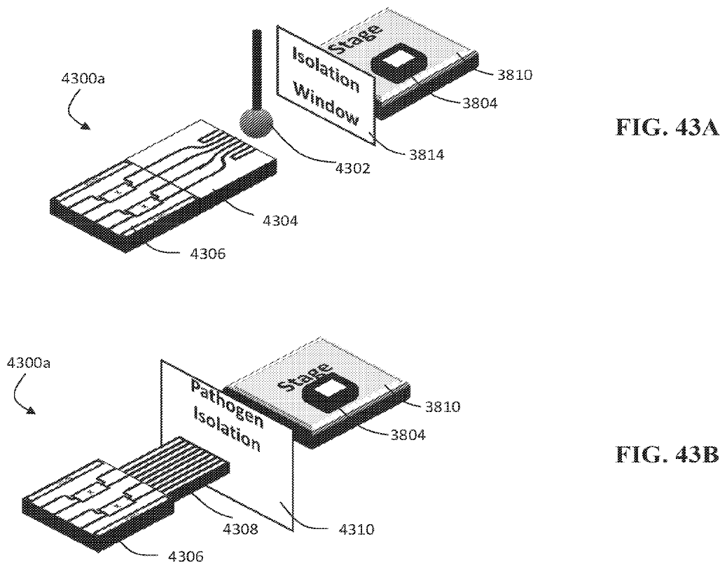

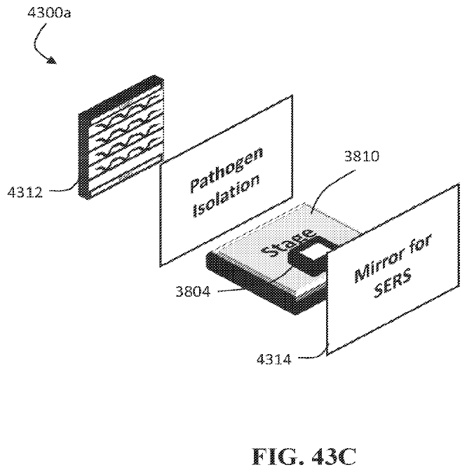

FIGS. 43A-43C illustrate embodiments of an alignment module. FIG. 43A illustrates an alignment module 4300a including one or more lenses configured to focus light between the interrogator 3802 and the cartridge 3804. For example, the lens can be a ball lens 4302 that can be adjusted in one or more axes (e.g., by an actuator). The alignment module 4300a may include an edge coupler array 4304 edge-coupled with a switch network 4306. FIG. 43B illustrates an alignment module 4300b including a switch network 4306 connected to a fiber array 4308. Note that some embodiments may include a pathogen isolation barrier 4310. This barrier can be used to isolate the cartridge 3804 and/or stage 3810 from the interrogator 3802. and/or alignment module 3812 (e.g., to avoid contamination if infectious pathogens are present, to allow for sterilization or cleaning if needed, etc.). The barrier 4310 can he transparent in the relevant wavelength range (e.g., UV-visible or infrared). FIG. 43C illustrates an alignment module 4300c that includes a phased array beam-steering chip or a MEMS beam-steering chip 3804. This embodiment may include a mirror 38 for surface-enhanced Raman scattering (SERS).

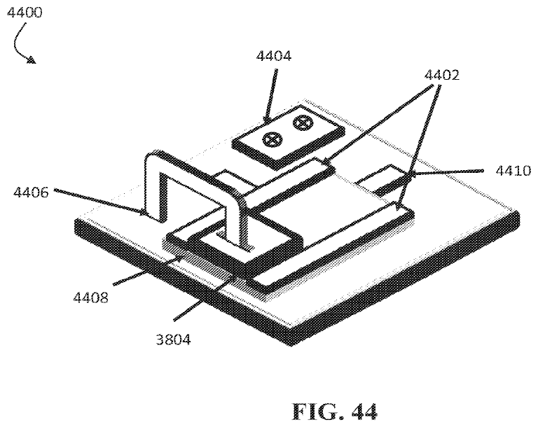

FIG. 44 illustrates an embodiment of a stage 4400, which enables the efficient and easy replacement and/or alignment of the cartridge 3804. Passive alignment features 4402 on the stage 4400 may be used to precisely align the cartridge 3804. In some embodiments, active micro-actuators are used to improve mechanical alignment. In some embodiments, a piezoelectric device 4410 may be used for mechanical alignment. Alignment that is not accomplished by the stage 4400 may be compensated for electro-optically in the alignment module 3812. For example, the alignment module 3812 can be configured to ensure that light passes from the interrogator 3802 to the sensor chiplet and then return to be detected (by the interrogator 3802). In some embodiments, the sensor chiplet includes and/or is coupled to a microfluidic cell. Further, the stage 4400 may include a microfluidic cell that accepts test chiplets, or connections for the microfluidic cell that may be included on the sensor chiplet. The stage 4400 may include a pump 4404 that can drive flow in the microfluidic cell. The stage 4400 may have a source of varying magnetic field to power magnetic stirring (e.g., via magnetic mixer 4406) in the microfluidic cell. The stage 4400 may be adapted to perform PCR and other reactions requiring temperature control (e.g., by a controller 4408) via temperature cycling provided by built-in thermoelectric heaters and/or coolers. Electrical connections may be built into the stage 4400 to power heaters or other electronics on the cartridge 3804 or sensor chip (via the cartridge 3804). The stage 4400 may be configured to accept multiple cartridges 3804. The stage 4400 may be configured to discard a cartridge 3804 after use and, in some cases, replace it with another one automatically (e.g., via a robotic arm, as described further herein).

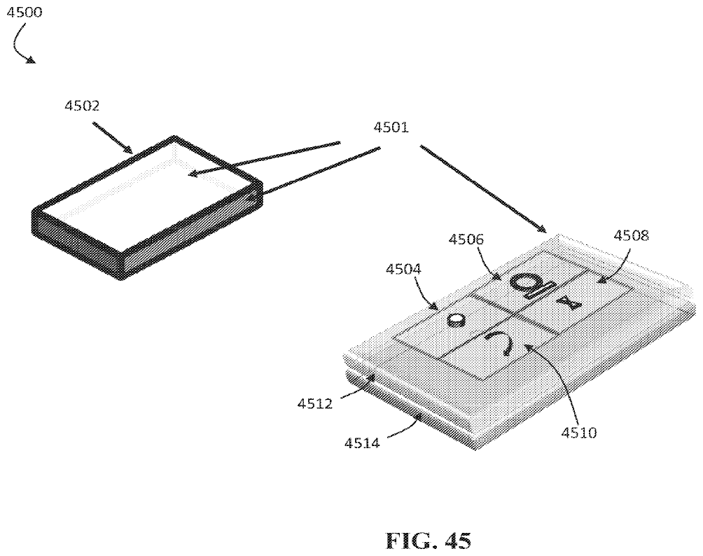

FIG. 45 illustrates an example cartridge 4500, which may be an assembly including a sensor chiplet and one or more microfluidic cells. In some cases, the housing of the cartridge 4500 can be configured to house any sensor chiplet and one or more microfluidic cells with compatible features and/or sizes. The cartridge 4500 may be configured to house a plurality of sensor chiplets and microfluidic cells 4501 For example, the housing 4502 can have mechanical alignment features so as to align with the interrogator 3802 and/or stage 3810. There may be multiple configurations of cartridges 4500, in which each configuration may support different types of sensor chiplets and/or microfluidic cells. The cartridge 4500 may have connection ports for electronic connections from the stage 3810. An electrical connection to cartridge 4500 may be made for electrochemical sensing simultaneously to and optical connection. The cartridge 4500 may have microfluidic input and/or output ports. The example cartridge 4500 includes a microfluidic cell, a one-way analyte input 4504, a photonic sensor array 4506, reagents 4508, and a mixing device 4510 (e.g., a magnetic input or output, pump input or output, etc.). The cartridge 4500 may be made of, at least in part, silicon, silicon nitride, porous silicon, thin film gold on SiO2, etc. or other chip material. The cartridge 4500 may be adapted to one of many labeled and label-free biosensing tests via a microfluidic cell 4512 on top of the sensing surface 4514.

As illustrated in FIG. 45, the microfluidic cell 4512 may be directly on top of the sensor chiplet 4514. In other embodiments, the microfluidic cell 4512 may be integrated into the cartridge and receive the sensor chiplet 4514. The microfluidic cell 4512 may be used to receive the analyte, which may be delivered through a sealable one-way input 4504 to ensure pathogen isolation, may deliver reagents 4508 to the sensing components on the sensor chiplet 4515, and/or may mix or flow (e.g., via mixing device 4510) the analyte to ensure sufficient and fast interaction between the analyte and the sensor chiplet surface 4514. An input and output port may be included to receive reagents 4508 or for pumping using an external pump on the stage 3810. The microfluidic cell 4512 may include a paper or other component to create flow using capillary forces. The microfluidic cell 4512 may apply heat by light (e.g., via plasmonic antennas) or micro-electric heaters (e.g., on the sensor chiplet 4514). The microfluidic cell 4512 may generate vortex mixing or include magnetic micro-mixing components (e.g., those made by Redbud Labs, Inc. of Research Triangle Park, N.C., USA). In some embodiments, the microfluidic cell 4512 is fabricated in many copies onto an entire water of sensor chiplets 4514, and then diced along with the underlying wafer. This enables wafer scale fabrication of the sensor chiplet 4514 and microfluidic cell 4512 together.

### Mechanical and User Interface Implementations

In the following, implementations of the integrated photonic biosensing systems are provided. Such implementations may include portable or tabletop systems and may be referred to as the “Pandemic Response Optical Biosensor Engine”, “PROBE”, or “photonic biosensing. platform”. For example, these photonics-based sensing systems and methods can be used as part of a rapid, point-of-care medical diagnostics platform.

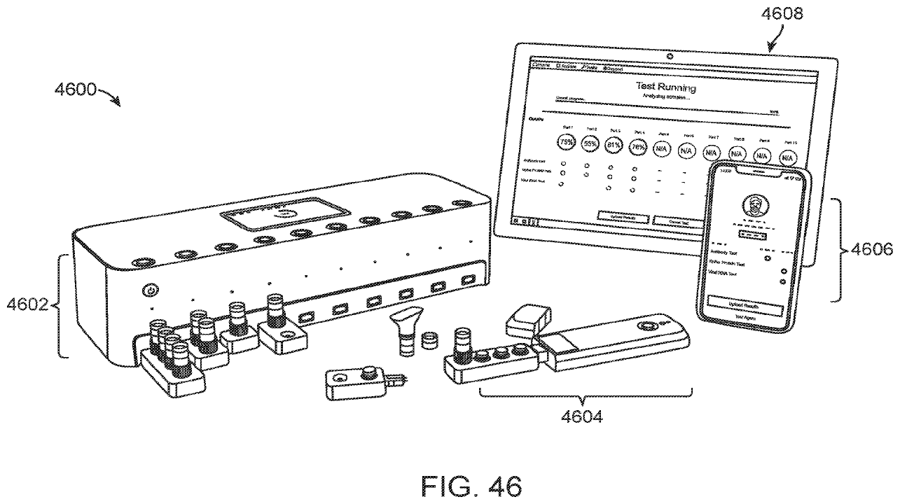

FIG. 46 illustrates various components associated with the photonic biosensing platform. As described in further detail below, the example platform 4600 can include tabletop apparatus 4602, handheld probe apparatus 4604. and display and related software 4606 to monitor and communicate real-time testing results. In some embodiments, the platform 4600 may include an additional interface 4608 for presenting the same or different information as the display 4606.

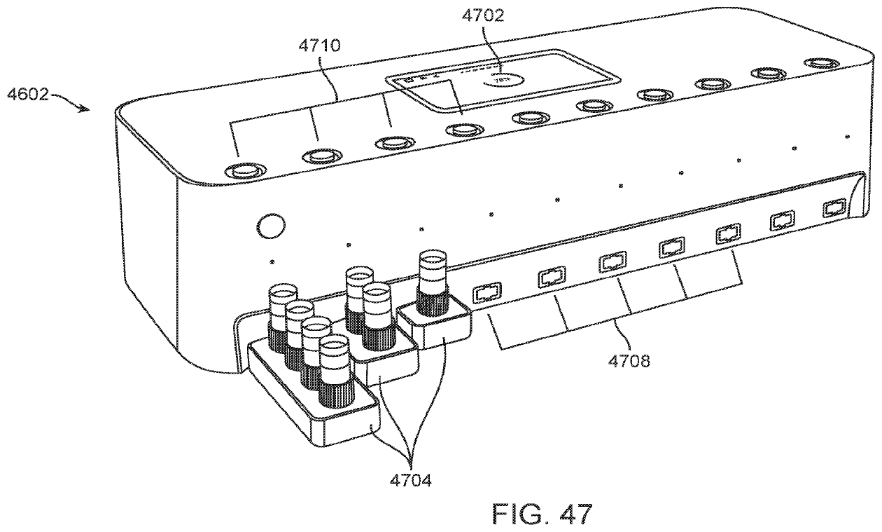

FIG. 47 illustrates the example tabletop apparatus 4602, including display 4702, disposable test cartridges 4704, apparatus input ports 4708, and buttons 4710. The example display 4702 can be configured to communicate testing results in real time or near real time to a user of the platform. The disposable cartridges 4704 can be used to test biological samples including, e.g., saliva, blood, urine, mucus, nasal swab sample, etc. The input ports 4708 can be configured to connect and align cartridges 4704 to the tabletop apparatus 4602. The tabletop apparatus 4602 is configured to interrogate the cartridges 4704 using the methods described herein. For example, a user can initiate the testing in a particular cartridge or set of cartridges 4704 by pushing the button 4710 associated with the input port. Note that the apparatus 4602 can be configured to test multiple samples (e.g., in respective cartridges 4704) in parallel.

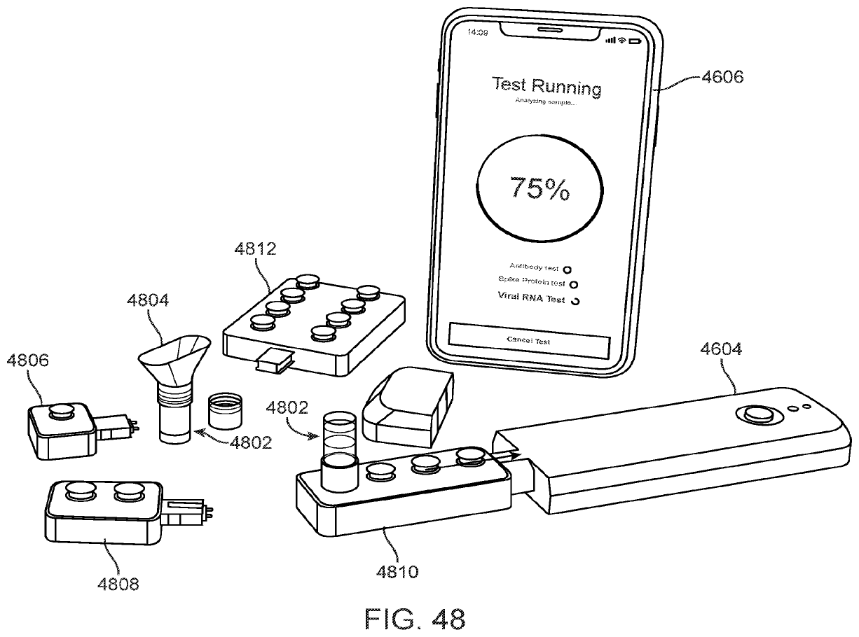

FIG. 48 illustrates the example portable apparatus 4604, and related components, and the display and related software 4606. The portable apparatus 4604 can be a handheld device configured to test a single cartridge 4704 at a time. In some embodiments, the apparatus 4604 can be configured to test a more than one cartridge 4704 at a time. As illustrated the portable apparatus 4604 has one input port for cartridge 4704. Each cartridge 4704 can hold one or more tubes 4802 to hold the biological sample. For example, cartridge 4806 can hold a single tube 4802, cartridge 4808 can hold two tubes 4802, cartridge 4810 can hold four tubes 4802, and cartridge 4812 can hold eight tubes 4802. One or more tubes 4802 of biological sample can be in contact with a sensor chiplet and/or a microfluidic cell, as described above. In some embodiments, a collection funnel 4804 can be fitted to the cartridge 4704 to facilitate collecting of the biological sample, The apparatus 4604 can include a communication module (e.g., via Bluetooth, Wi-Fi, RFC, radio, etc.) configured to communicate with the mobile device 4606. An application on the mobile device 4606 can be configured to process information from the apparatus 4604 to monitor and/or display the test status and/or results.

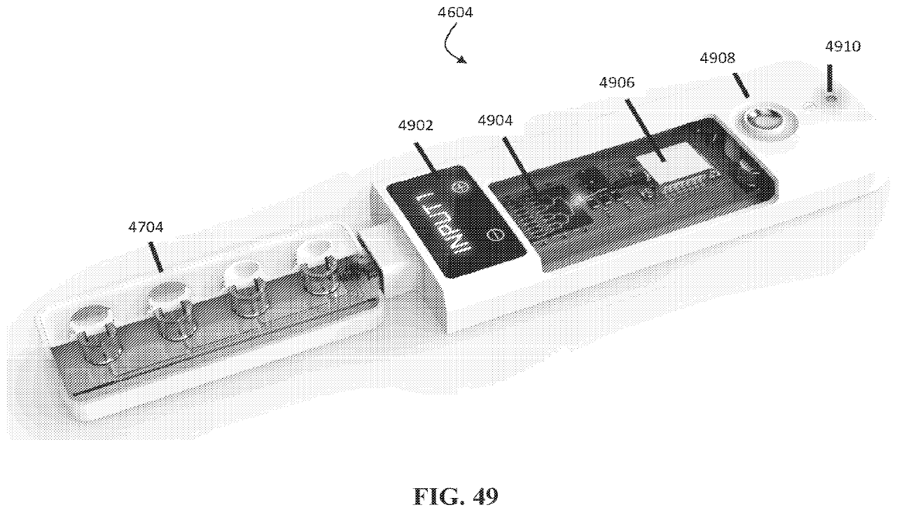

FIG. 49 illustrates the example handheld apparatus 4604 optically coupled to cartridge 4704. The apparatus 4604 includes a display 4902, one or more waveguides 4904, a light source 4906, a heating pad 4908, and a status indicator 4910. The display 4902 can be configured to confirm proper insertion (e.g., including alignment) of the test cartridge 4704 into the apparatus 4604. For example, the display 4902 can indicate with a light (e.g., LED), color, text, etc. whether the cartridge 4704 is properly fitted. As described above, the light source 4906 provides a light to the waveguides 4904 to be used for sensing (e.g., by the sensor chiplet) at the test cartridge 4704.

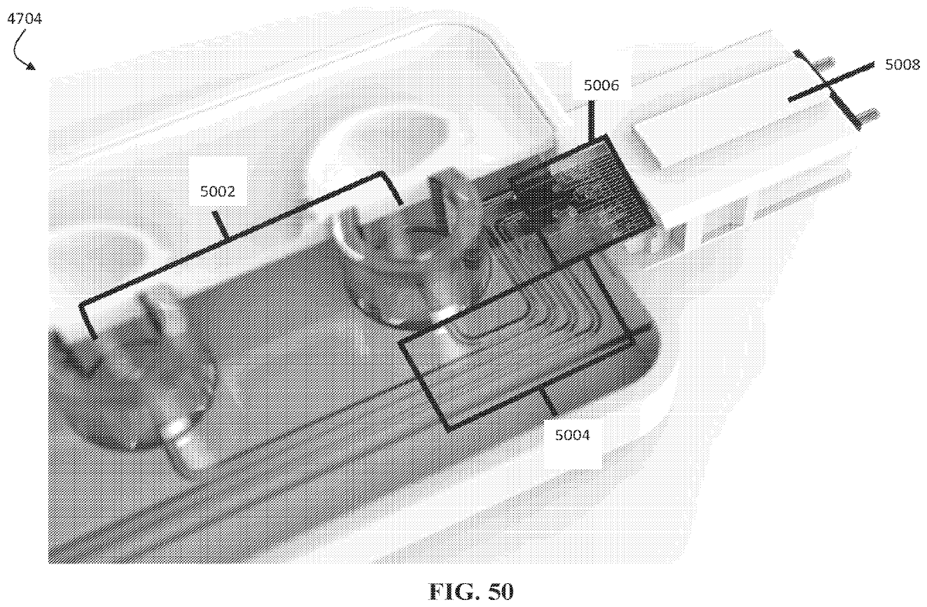

FIG. 50 illustrates a close-up view of example cartridge 4704 configured to be inserted into the tabletop apparatus 4602 or handheld apparatus 4604. In some examples, the disposable test cartridge 4704 may include lyophilized CRISPR compounds (e.g., CAS 9, 12, 13, etc.) to facilitate detection. The test cartridge 4704 includes a reservoir 5002 for holding a test sample, one or more microfluidic channels 5004 that transport test sample towards the active photonics-based sensing area, the sensing area 5006 where photonics-based biosensing is performed on samples, and a connector 5008 for mating and alignment with the interrogator apparatus (e.g., the tabletop 4602 or handheld apparatus 4604).

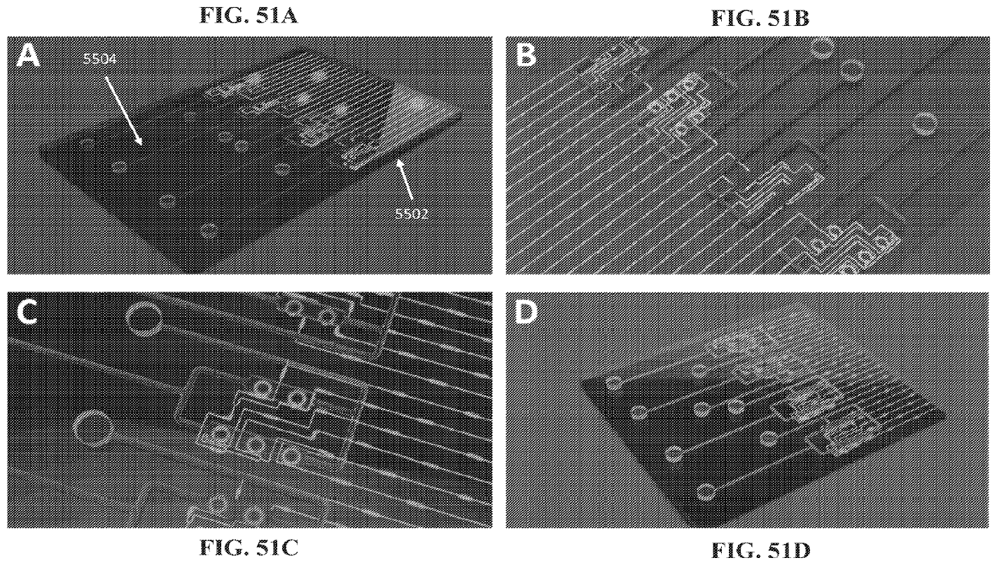

FIGS. 51A-51D illustrate four implementations of the biosensor chip 5102 with microfluidics 5104 that may be used within the test cartridge 4704. Each chip can have multiple sensing channels and can accept one or more types of reagents, e.g., saliva and blood.

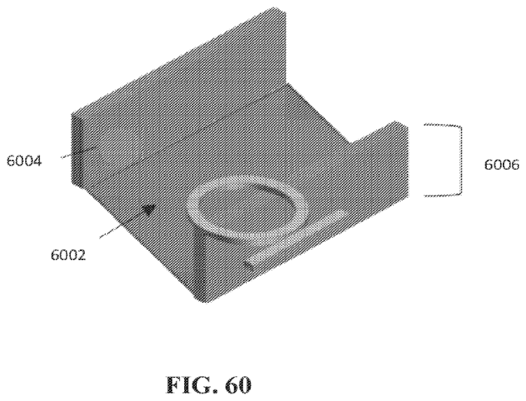

In various embodiments, the microfluidics channels used to transport the analyte in the sensing systems and methods described herein can be configured to facilitate detection of the sensing target, biological marker, pathogen of interest, etc. For example, the analyte including at least one of reporter probes, sensing targets, biological markers, pathogens, etc. may flow perpendicular to the waveguide in a microfluidic channel to maximize interactions associated with the sensing protocols outlined above. Forcing the analyte past the waveguide may increase probability of any number of the described interactions in the sensing schemes described above (e.g., binding, cleaving, etc.). FIG. 60 illustrates an example microfluidic channel 6002 that transports analyte to the waveguide (e.g., of the sensing area, as illustrated in examples of FIGS. 51A-51D). In some embodiments, an amplification agent (e.g., sensing amplifiers) 6004 may be applied to the channel 6002 to improve sensitivity to a particular characteristic of the biological sample. In some embodiments, the channel height 6006 may be manipulated to improve sensitivity. For example, the microfluidic channel height 6006 may be optimized to promote interaction between the analyte and the waveguide. In some cases, the microfluidic channels may be made using oxide etching of the cladding oxide on silicon photonic chips and sealing the channel overhead (e.g., via flat silicon bonded to silicon in a later step). Varying the thickness of oxide may control the resulting microfluidic channel height 6006. Additionally or alternatively, deep trenches etched in the silicon chip may be used as additional channels or fluid reservoirs to store analyte prior to interaction with the waveguide or after it flows past the waveguide.

### Biosensing Methods

Methods and systems related to biosensing with a photonic waveguide on a sensing chip or fiber are described herein. The sensing chips or fibers may be made using silicon, silicon nitride, silicon dioxide or any other commonly used waveguide materials. In some embodiments, the methods/systems described herein include additional known amplification techniques.

In some embodiments, the sensor chiplet is adapted to perform a labeled and label free biosensing tests. In some embodiments, the sensor chiplet performs biosensing via in plane light propagation through waveguides. In some embodiments, the sensor chiplet performs biosensing via reflections (such as Surface Enhanced Plasmon Resonance) or other out-of-plane interactions.

In sonic embodiments, biosensing is performed on a surface in an electronic, optical, MEMS, or optoelectronic device. General sensing techniques include but are not limited to using a doped optical waveguide or electrodes near a waveguide to sense the optical change or resistance change, respectively, after a binding or cleavage event. In some examples, optical changes may be detected using surface plasmon resonances, Mach-Zehnder interferometers, spiral waveguides, Bragg gratings, and/or photonic crystals or magnetic dielectric mirrors. In some embodiments, the waveguide is configured to detect a signal based on wavelength dependence or a wavelength resonance. In some embodiments, the interferometer is an unbalanced Mach-Zehnder Interferometer.

In some embodiments, a microfluidic cell is placed on top of the sensing surface. Such a microfluidic cell can be used to control flow of reagents, sample, and other components to and from the sensor chiplet.

As described herein, integrated photonic sensors can be used to detect changes to biomolecules, e.g., due to binding or cleavage interactions, that are immobilized on or near a waveguide. The evanescent field emanating from the waveguide is used to sense a change in the biomolecule.

In some examples, optical changes may be detected using surface plasmon resonances, Mach-Zehnder interferometers, spiral waveguides, Bragg gratings, and/or photonic crystals or magnetic dielectric mirrors.

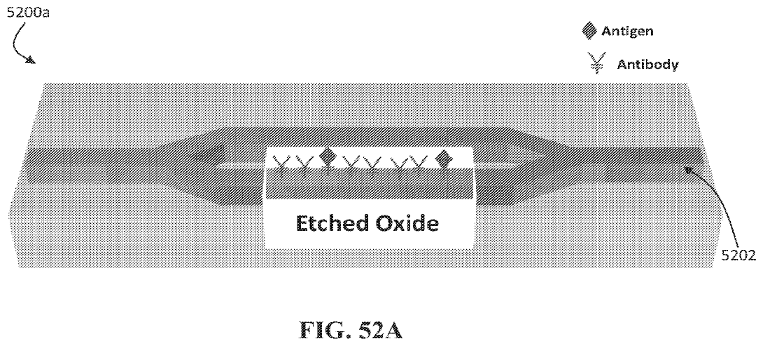

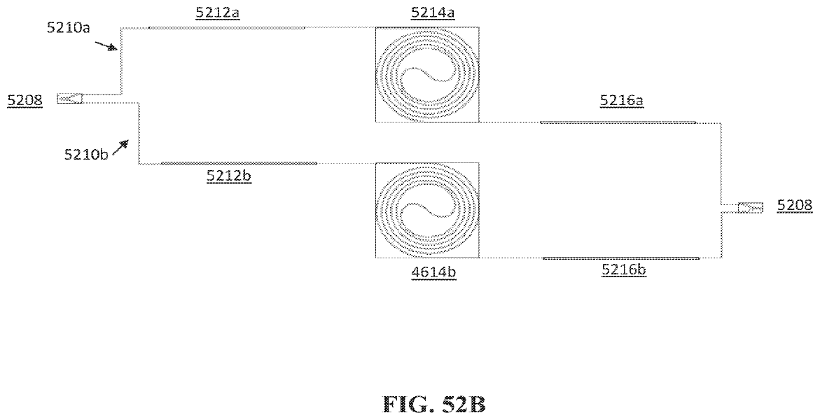

In FIG. 52A, the sensor chiplet 5200a (also referred to as an integrated photonic sensor) can include a waveguide 5202 including antibodies in at least one channel. These integrated photonic sensors rely on the binding of antigens from the analyte to antibodies that are immobilized on or near the waveguide 5202. The evanescent field emanating from the waveguide can be used to sense the refractive index change due to the presence of antigen after binding. If the waveguide is one arm of an interferometer (for example Mach-Zender or Michelson Interferometer), as shown in FIG. 52A, a phase change is introduced by the change in the effective refractive index experienced by the light passing through the waveguide, thus causing a change in intensity output from the interferometer.

FIG. 52B illustrates a Mach-Zender Interferometer (MZI)-type sensor. The sensor 5200c can include a Y-splitter 5208 in which a first channel 5210a includes a Bragg reflector 5212a, a spiral waveguide 5214a, a Bragg reflector 5216a. The second channel 5210b includes a Bragg reflector 5212b, a spiral waveguide 5214b, a Bragg reflector 5216b.

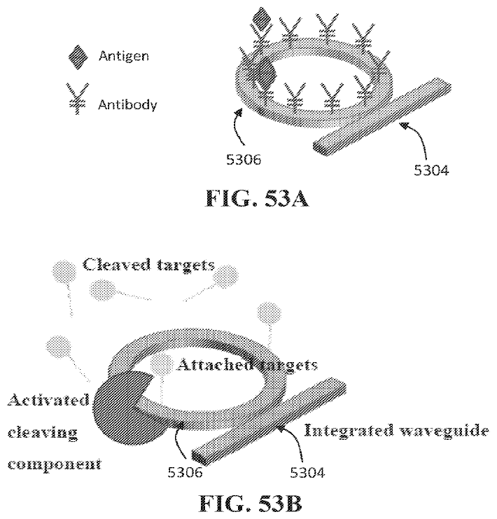

FIG. 53A illustrates an example method where an antigen binds to an antibody immobilized on a ring resonator. FIG. 53B illustrates an example method wherein a cleaving agent cleaves a reporter probe immobilized on a ring resonator. the waveguide 5304 is part of a ring resonator 5306, the waveguide 5304 can detect a change in the resonance of the ring 5306, which will shift after a binding or cleavage reaction with one or more molecules. Alternatively, if the antigen or cleaved element is absorptive in wavelengths that are guided in a given integrated photonic waveguide, light intensity may simply be measured after passing through the waveguide.

### Binding Assays

In some embodiments, an analyte can be detected through binding to a biomolecule immobilized on or near a waveguide. For example, binding of antigens to antibodies that are immobilized on or near a waveguide can be detected by an integrated photonic sensor. The evanescent field emanating from the waveguide is used to then sense a refractive index change due to the presence of antigen after binding.

In another embodiment, biosensing is performed by a biological marker (e.g. virus antigens, antibodies, etc.). The biological markers may be immobilized at or near the waveguide.

In some embodiments, whole pathogen detection is performed. The pathogen may be bound to a waveguide by functionalizing the waveguide with antibodies that capture the pathogen. However, because the refractive index of a virus, for example, is in the range of 1.4-1.5 and water is 1.33, it can be hard to detect a single viral particle. To increase the signal, an optically active component may be attached to the pathogen. In some embodiments, a plasmonic particle or other complex with strong optical properties may be attached to the pathogen by functionalizing the nanoparticle with antibodies for the pathogen. The pathogen may be bound to a waveguide by functionalizing the waveguide with antibodies that capture the pathogen.

In some examples, RNA/DNA is first functionalized with a reporter probe, then it may bind to conjugate DNA/RNA attached to the waveguide. The reporter probe may have a sequence which precisely binds the DNA/RNA (single strand). When the reporter probe is away from the waveguide, the binding site is therefore closed off. When the reporter probe connects to the sensing target (e.g. viral DNA) it unfolds, and the binding site is revealed.

The biological markers may be in solution and bind to the waveguide in any number of ways. The waveguide may then detect the refractive index change due to the presence of the biological marker at or near the waveguide. Alternatively, if the biological marker is optically active in the region the waveguide operates at, light intensity may simply be measured after passing through the waveguide.

### Cleavage Assays

In some embodiments, a component of a sample can be detect by directly or indirectly resulting in a cleavage reaction which is detected by the sensor chiplet. FIG. 53B illustrates an example process wherein the cleaving agent cleaves a reporter probe from a waveguide.

In one example, a waveguide (e.g. associated with a ring resonator) is functionalized to immobilize reporter probes (e.g. RNA strands). Next, a cleaving component (e.g. a CRISPR enzyme) that may interact with the reporter probes and a sensing target of interest may be combined with analyte carrying the sensing target. Herein, the use of sensing target is intended to include any biological marker. This includes but is not limited to RNA, DNA, a molecule, an enzyme, an antigen, an antibody, a pathogen, etc.

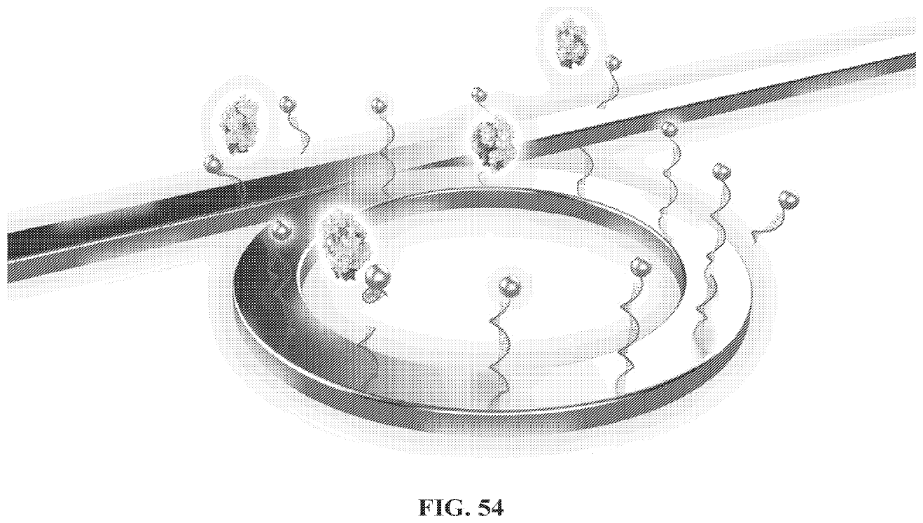

FIG. 54 illustrates one possible testing mechanism used by the PROBE apparatus; it utilizes A—waveguide, B—ring resonator, C—functionalized nanoparticles (e.g. reporter probe with optically active component), and D—sensing agents capable of cleaving the nanoparticles from the ring resonator. In one example, a waveguide (e.g. associated with a ring resonator) may be functionalized to immobilize reporter probes (e.g. RNA strands). These reporter probes may be linked to an optically active component (e.g. plasmonic nanoparticle, quantum dot, molecule, etc.) to enhance their optical effect on the waveguide and/or for downstream detection. Next, a sensing agent or cleaving component (e.g. a CRISPR enzyme) that may interact with the reporter probes and a sensing target of interest (e.g. virus RNA/DNA) may be combined with analyte carrying the sensing target (e.g. viral RNA from patient sample introduced into test cartridge). In some cases, the sensing agents are activated to cleave the functionalized nanoparticles only if they encounter biological mated al associated with a positive test result (e.g. viral RNA). The cleaving component may be activated, thereby indiscriminately cleaving the functionalized nanoparticles. In some examples, if the reporter probes attached to the waveguide are removed, an optical change in the system may be detected in various ways. In one example, cleavage of the probes from the waveguide may result in a change in the refractive index of light guided within the waveguide; this change in refractive index may be detected using various spectroscopic techniques (e.g. resonance, interference, or absorption, etc.). Additionally or alternatively, the optically active component (e.g. plasmonic nanoparticle, quantum dot, molecule, etc.) attached to the reporter probes may be cleaved along with the reporter probes. The presence of these cleaved optically active components may be detected downstream from the waveguide using various techniques. Other known techniques for facilitating interactions between the waveguide and sensing targets, reporter probes, biological markers, pathogen, etc. (e.g. toehold switch) may be implemented in addition to or as an alternative to the described techniques.

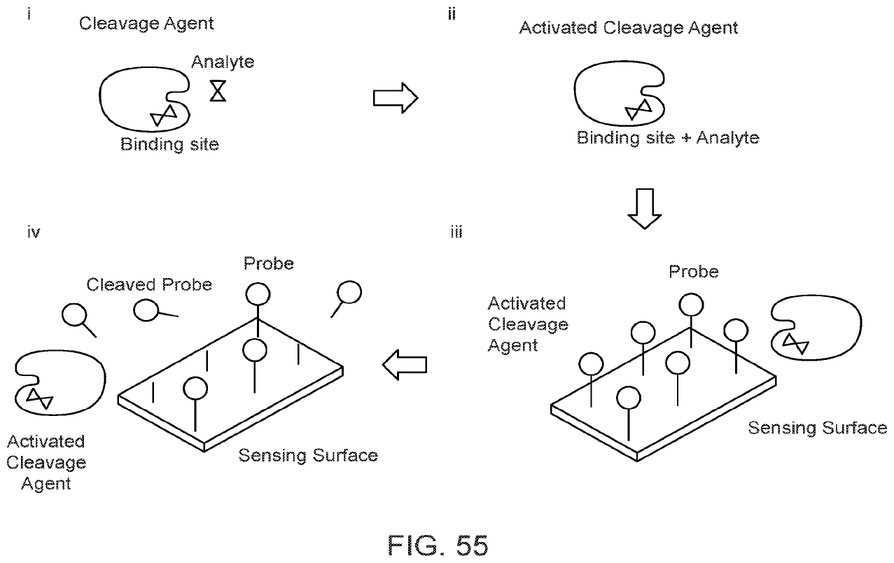

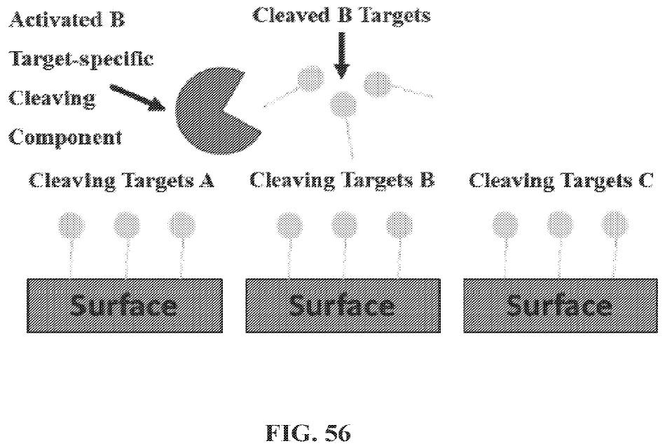

In some embodiments, as illustrated in FIG. 55, the cleaving component is designed to be activated only when it detects the sensing target of interest. Once the cleaving component is activated, it cleaves probes from the sensing surface, leading to a detectable signal (via resonance, absorbance, interference resistance changes or other detectable changes near the sensing surface).

In some embodiments, the cleaving component binds to the sensing target of interest. The cleaving component may be activated, thereby indiscriminately cleaving both the sensing target and immobilizer probes.

Various cleaving components (e.g. CRISPR enzymes activated by target RNA, or other enzymes activated by an analyte of interest) that may cleave the reporter probes, removing them from the surface, when an analyte of interest binds to or is otherwise detected by the cleaving agents in solution.

In some embodiments, the probes are engineered to enhance the signal generated by cleavage events, which is distinct from other techniques where binding of analyte to the surface directly generates a signal. The readout maybe done by immobilizing the probes on the surface of waveguides, such that the evanescent field interacts with the probes, but any surface method or any combination of surface methods (e.g. electrical and/or optical) may be used including transistors, nanopores, surface plasmon resonant thin films or particles, surfaces used for SERS spectroscopy, or electrical impedance (e.g., resistance) based sensors. In some embodiments, a high contrast cleavage detection system, where there is both a cleaving component that is either the analyte of interest or has a specific detection mechanism for the analyte of interest, and a solid state probe that is functionalized onto a sensing surface (e.g. a waveguide, plasmonic thin film, etc.), is used.

In some embodiments, the cleavage event is caused by the analyte of interest or may be facilitated via a chemical in solution and/or from electromagnetic radiation (e.g. UV light). The method may be used directly to detect any effect that causes the probe removal; this includes light, heat and other changes in the environment generally or locally that can cause the probe to detach. In a nonlimiting example, probes may contain UV cleavable linkages or heat-disassociated bonds. For sensing analytes in solution that are exposed to the surface, the cleavage event may be activated by a chemical or enzyme associated with the sensing target. In one non-limiting example, the cleaving component may be an enzyme (e.g. CRISPR, a Toehold Switch RNA detection produced Enzyme or protein) that may cleave reporter probes (e.g. RNA strands) immobilized on the surface of an electronic, magnetic, MEMS, optical, or optoelectronic device. The cleaving component may be activated when it detects the sensing target of interest in solution, thereby cleaving the immobilized reporter probes.

In some cases, the immobilized reporter probes consist of an optically-active and/or conductive or magnetic component, which may facilitate detection of this cleavage event (e.g. via the optical signal or a change in resistance at an electrode described above). This cleavage may be sensed directly where it happens (e.g. by a change in response of a ring resonator/optical waveguide where the reporter probes were immobilized prior to cleavage) or the cleaved products (e.g. the cleaved reporter probes migrate away from the surface for detection elsewhere in the system). The cleaved products may migrate to and bind to a sensing surface via diffusion or mixing. In some examples, the cleaved product may be designed for strong binding affinity to the sensing surface (e.g. surface functionalized gold particles functionalized with biotin designed to bind to sensing surface functionalized with Streptavidin.)

This method may also be used to determine or sense activity or reaction kinetics associated with a biomolecule or enzyme even if the reaction is reversible. For example, if the surface is functionalized with an agent the biomolecule reacts with, a binding event associated with this reaction may be detected (e.g. via optical resonance shift etc.), and if the complex falls apart or is broken, this can be detected as a cleavage. The contrast can be increased by labeling the component that is added from solution using a gold nanoparticle or otherwise optically/magnetically/electrically, active label that interacts strongly with the surface.

Additionally or alternatively, various enzymes may be attached to various surfaces and their activity may be monitored separately using the optical and/or electronic interactions described above. For example, an optical system may include multiple ring resonators where each ring resonator may be functionalized with a different enzyme (e.g. CRISPR CAS 12, CAS 13, etc.). These various cleaving components may be designed to be activated only when they are exposed to their specific sensing target of interest as shown in FIG. 55. Once activated, each cleaving component, tethered to the sensing surface, may cleave only cleave probes in its direct vicinity. This may lead to a change in the response (e.g. plasmon resonance optical readout or electronic transistor readout) of only the surface where the cleavage occurred (see FIG. 56). This may permit different regions or different surfaces to detect different analytes in the same sample without any interference and without the need for any microfluidic or other physical separation. In the nonlimiting example of RNA sensing with ring resonators, different resonators on a chip may be functionalized by a CRISPR enzyme carrying a different crRNA sequence, allowing each ring to become a sensor for that specific sequence when all the rings are exposed simultaneously to the same analyte. This can work with any version of the High Contrast Cleavage approach described.

Alternately, instead of attaching different enzymes or other cleaving agents with different target analytes to different sensing surfaces, the sample fluid may be split up into separate chambers, each containing a different cleaving agent (in a dried state or added via a different fluid input channel/port) with a different target analyte. This allows testing of the same sample for different analytes in parallel without interference. It may also be arranged in a serial fashion, where the sample flows first over a sensing surface where the microfluidic chamber contains the first cleaving agent, then flows into a chamber with the second cleaving agent, and so on (e.g. each chamber containing 1 or more sensing surfaces with cleavable probes). Using the two above described techniques (separate optical system with distinct enzyme, splitting sample fluid) may be useful for both redundant testing (e.g. for the same virus) by increasing sensitivity and/or specificity and multiplexing tests for multiple pathogens which may be advantageous for facile widespread testing.

### Toehold Switch Assay

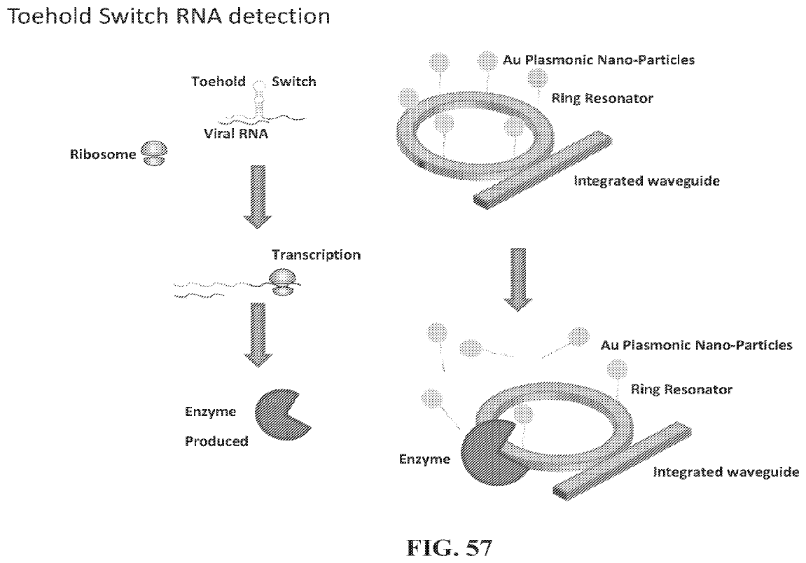

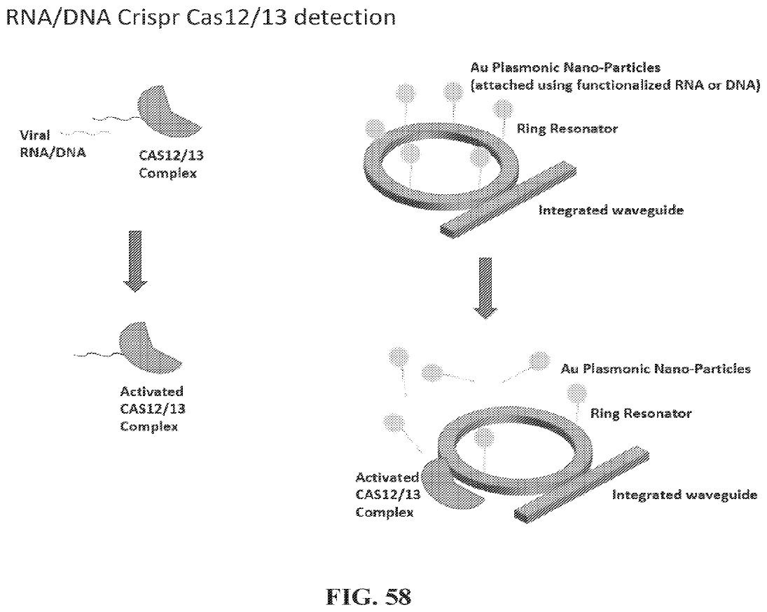

Shown in FIG. 57 and FIG. 58 are diagrams of methods developed for integrating diagnostic tests with waveguides and other integrated photonic components.

In FIG. 57, we illustrate a method for RNA detection using a toehold switch RNA approach based on what is demonstrated in Rapid, low-Cost Detection of Zika Virus Using Programmable Biomolecular Components K Pardee et al. When the hairpin is opened by the target RNA binding to the toehold RNA, the ribosome binding site is exposed and the reporter protein sequence may be transcribed by a ribosome in solution. The reporter protein is chosen such that it may cleave the bonds that immobilize a plasmonic nanoparticle (or other complex with a strong optical response) from the waveguide surface. Again, the effect on the guided light within the waveguide may be detected using one of the methods described above (resonance, interference, or absorption).

In some examples, sensing target detection (e.g. RNA) may use a toehold switch RNA approach, as shown in FIG. 57. For example, the hairpin may be opened by the target RNA binding to the toehold RNA, thus the ribosome binding site may he exposed and the reporter protein sequence may be transcribed by a ribosome in solution. In some cases, this approach may generate an enzyme or other protein that may act as an input for the sensing approach, The reporter protein is chosen such that it may cleave the bonds that immobilize an optically active component from the waveguide surface. Thus, an effect on the guided light within the waveguide may be detected using one of the methods described above (e.g. resonance, interference, absorption, etc.)

### CRISPR Assay

In one non-limiting example, the cleaving component is a CRISPR CAS-13 complex which cleaves all nearby RNA, including the RNA reporter probes immobilized on the waveguide.

In FIG. 58, we illustrate a method for DNA and RNA detection using CRISPR and a waveguide. A waveguide may be functionalized using standard methods with DNA or RNA strands. These strands may be linked to a plasmonic (e.g. gold) nanoparticle, quantum dot or another molecule to enhance their optical effect on the waveguide. A CRISPR enzyme such as CAS12 (for DNA) or CAS13 (for RNA) carrying the relevant sequence may be combined with analyte carrying the RNA or DNA of interest. When the CAS protein binds the RNA or DNA of interest it may be activated and used to cut multiple RNA/DNA strands nonspecifically. If the DNA/RNA strands attached to the waveguide are cut, the effect on the guided light within the waveguide can be detected using one of the methods described above (resonance, interference, or absorption). See Integrated Micropillar Polydimethylsiloxane Accurate CRISPR Detection (IMPACT) System for Rapid Viral DATA Sensing, Kenneth N. et al. for a similar approach. In some CRISPR implementations, after a sensing target is identified, the cleaving component may cleave a cluster of enzymes connected with an RNA/DNA scaffold. These enzymes may become activated and may cleave probes from the photonic waveguide. In some cases, they may not be enzymes but instead some type of particle that binds to the waveguide. This binding changes the local refractive index. The binding site is therefore hidden when they are connected to the cluster. Thus, the binding site may only be opened when the particle is cleaved.

The processes above describe several possible sensing techniques using a photonic waveguide, as taught herein. These processes may be further performed with or without common techniques associated with biosensing (e.g. target amplification). Other known techniques for facilitating interactions between the waveguide and sensing targets, reporter probes, biological markers, pathogen, etc (e.g. toehold switch) may be implemented in addition to or as an alternative to the described techniques.

### Further Biosensing Embodiments

The target of interest may first be chemically amplified using techniques including but not limited to PCR or RT-LAMP or RPA.

In some cases, reverse transcriptase may be used to convert RNA to DNA. This may allow for DNA sensing systems like PCR or CRIPSR CAS-12 to be implemented. For PCR, the sensing protocol may include emitting light into the analyte using vertical grating couplers or an evanescent field and then observing fluorescent response either using external or on-chip optics and photodetectors.

In another aspect of the present disclosure, a chemical reaction on the surface of an optical, electronic, magnetic, MEMs or optoelectronic device may be catalyzed. In one example, a chemical reaction at a waveguide may be catalyzed on a waveguide via an evanescent field associated with the waveguide. In some cases, the chemical reaction may be controlled via integrated photonics (e.g. by toggling the light on and off or switching between different input wavelengths) to activate chemical reactions selectivity (e.g. which reaction, where the reaction occurs, when the reaction occurs, etc.). Additionally or alternatively, reaction kinetics could be further controlled by controlling the intensity and/or wavelength using components such as ring resonators, optical switches, photonic crystals, Bragg gratings, LEDs, and lasers which are capable of introducing and controlling high-intensity light across a range of wavelengths. MEMs components may be fabricated either instead of or in complement to other components in order to control chemical reactions near the surface, induce mixing, induce polymer folding, induce strain in the surface or in polymers attached to the surface etc. In all cases, sensing may be done in parallel or serially as chemical reactions are occurring/being catalyzed/controlled.

In one implementation of High Contrast Cleavage Detection, an antibody, antigen or another analyte (which itself may be a complex of the target analyte and another molecule) may act as a bridge to combine two or more separate molecules into a cleaving agents which goes on to by an input to the sensing method as described above. Additionally a cleavage agent may be designed with a blocked active site such that the blocking element can disassociate in the presence of the correct analyte or when some change is sensed (pH, temperature, etc.), again working as an input to the sensing method.

### Optically Active Components

If the reporter probes attached to the waveguide are removed, an optical change in the system can be detected in various ways. In one example, cleaving the reporters from the waveguide may result in a change in the refractive index of light guided within the waveguide; this change in refractive index may be detected using various spectroscopic techniques (e.g. resonance, interference, or absorption, etc.). Additionally or alternatively, the optically active component (e.g. plasmonic nanoparticle, quantum dot, molecule, etc.) attached to the reporter probes may be cleaved along with the reporter probes. The presence of these cleaved optically active components may be detected downstream from the waveguide using various spectroscopic techniques (absorption, photoluminescence, fluorescence, etc.).

These reporter probes may be linked to an optically active component (e.g. plasmonic nanoparticle, quantum dot, molecule, etc.) to enhance their optical effect on the waveguide. Further, anything being captured by an antibody may be enhanced by attaching an optically active probe to it.

### Reaction Kinetics

Several methods to increase the likelihood of interaction between the waveguide and analyte containing sensing targets, reporter probes, biological markers, pathogens, etc. are described. In one example, optical trapping (e.g. using strong electric field near waveguide or other photonic structure, similar to optical tweezers) to trap the sensing target at or near the waveguide.

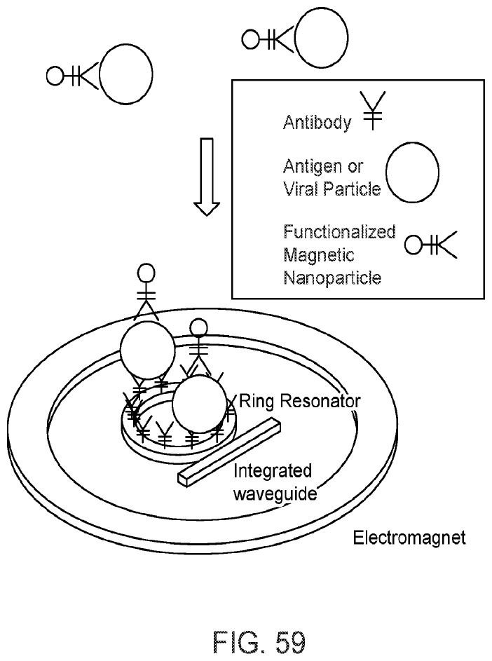

Additionally or alternatively, magnetic nanoparticles may be bound to the sensing targets, biological markers, or pathogens of interest. The sensing target, biological marker, or pathogen of interest may then be drawn to the sensing waveguide using a magnetic field applied externally or on the sensor. FIG. 59 further illustrates how attaching a magnetic particle to a sensing target may direct the sensing target to the waveguide via an applied magnetic field. By binding magnetic nanoparticles to the molecule or pathogen of interest, the complex may then be drawn to the sensing waveguide using a magnetic field, applied externally or via an electromagnet fabricated directly onto the sensor chiplet. This method may be combined with any diagnostic scheme, including those discussed above.

Additionally, one or more plasmonic antennas (e.g. a bowtie) may be fabricated on the chip such that local light-induced heating causes mixing via convection.

### Integrated Photonic Assemblies for Biosensing

In various embodiments, the biosensing systems and methods can include multi-photonic-chiplet (MPC)-based point-of-care (POC) diagnostic biosensors for multiplexed, label-free biosensing. Current lab-on-a-chip optical biosensors transduce the nature and concentration of analyte of interest into an output signal by sensing the change in the refractive index of the optical waveguide, This detection mechanism has been achieved through a variety of optical phenomena based on the sensor configurations including surface plasmon resonance (SPR) sensors, surface-enhanced Raman scattering (SERS), photonic crystal-based gratings, micro-ring resonators, or unbalanced Mach-Zehnder interferometer (UMZI) structures. While decades of research in this area has drastically advanced the sensitivity and specificity of these commercially-available sensor technologies, realization of compact, inexpensive sensors for multiplexed sensing of biological analytes applicable to point-of-care diagnostics has been elusive. The present systems and methods aim to provide such benefits. In particular, the present disclosure discusses in part a compact multi-photonic-chiplet (MPC)-based point-of-care (POC) diagnostic biosensor that could provide an inexpensive, re-usable, and scalable solution for simultaneous sensing of an array of biological analytes with enhanced specificity and sensitivity of detection.

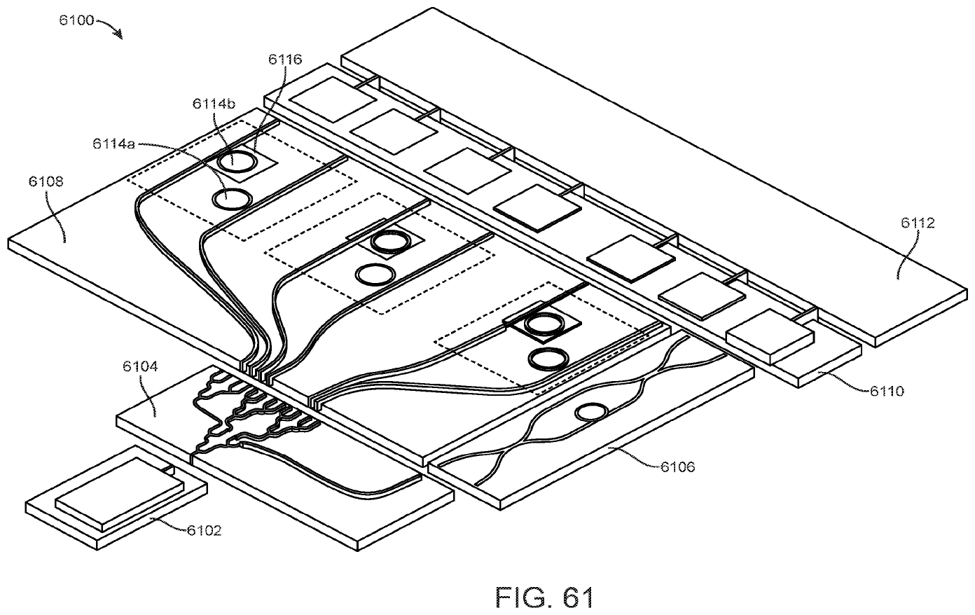

FIG. 61 illustrates an example implementation of the multi-photonic-chiplet (MPC)-based optical biosensor assembly 6100 for multiplexed sensing of analytes. This example assembly 6100 includes one or more multi-platform integrated opto-electronic chiplets. The chiplets can include an optical source 6102, a splitter network 6104, a frequency discriminator 6106, an array 6108 of sensing elements, a photo-diode array 6110, and read-out electronics 6112. The biosensor assembly 6100 may permit photonic chiplets having different elements of the sensing system (e.g., source, photodetectors (PD), and/or ring resonators) to be used in tandem. Each sensor element (labeled S1, S2 . . . Sn) of the sensor array 6108 may include two identical ring resonators 6114a, 6114b pumped by a tunable optical source. One of the resonators 6114b of the sensing element may be exposed to a biological analyte 6116, depicted as the shaded region around the ring, while the other resonators 6114a may be used as a reference. The optical source 6102 may be split across an array 6108 of sensing elements, enabling simultaneous or near simultaneous sensing of two or more analytes. This has the benefit of enabling each photonic component to be realized in the photonic platform of choice. For instance, this includes but is not limited to the currently foundry-friendly materials, e.g., silicon, silica, silicon nitride for the ring resonators, splitters, and the frequency discriminator, while the optical source and the photodetectors may be realized in silicon, or any III-V platform. Such flexibility may enable customization of individual components of the sensor from a myriad of photonic platform to suit the requirements of the sensing application and/or environment.

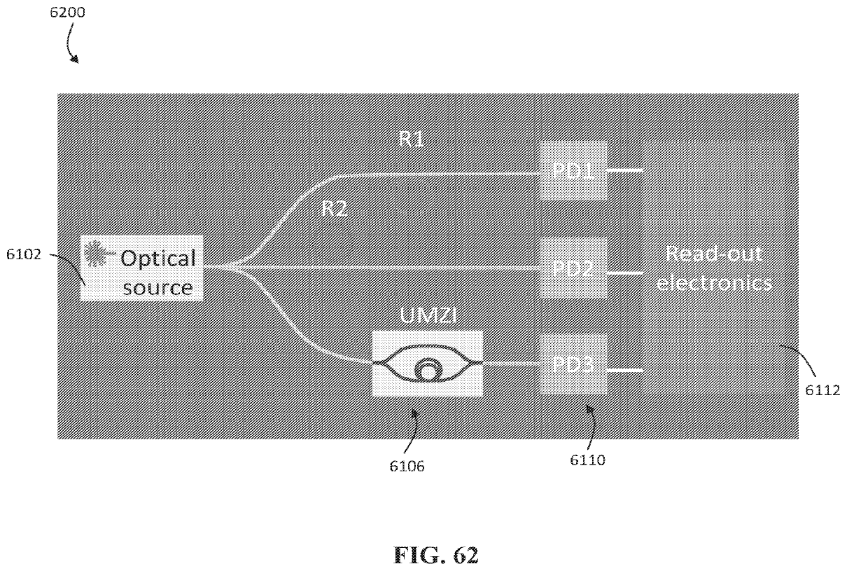

FIG. 62 depicts an example layout of the photonic biosensor 6200 with a single sensing element. This single, illustrative configuration may include two identical ring resonators R1, R2, an optical source 6102, a frequency discriminator 6106, PDs 6110, and read-out electronics 6112. In this example, the use of reference resonator element or elements (e.g., R2) may eliminate common-mode noise sources, e.g., thermal or vibration noise.

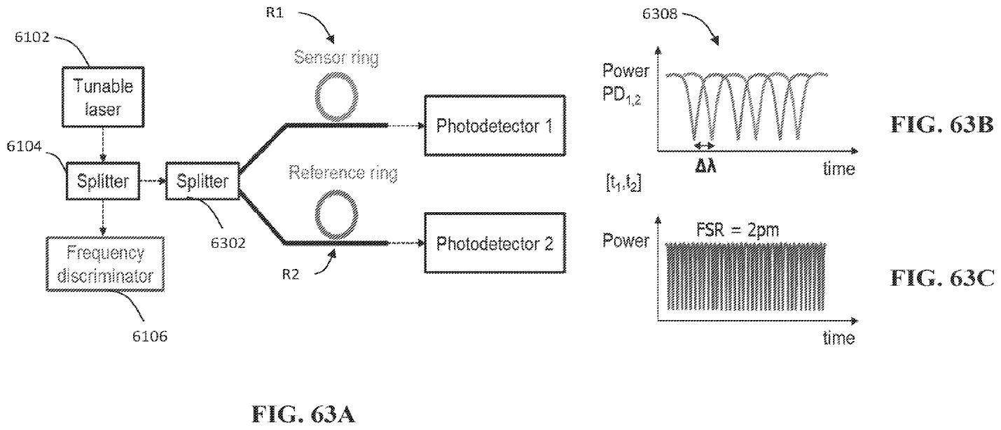

FIGS. 63A-63C illustrates the functionality of the sensing elements and frequency discriminator. FIG. 63A provides representation of the light source 6102 (e.g., a tunable laser) coupled to at least one splitter 6104. The first splitter splits the light between a frequency discriminator 6106 and a second splitter 6302. The second splitter 6302 provides the light to a first waveguide coupled to the sensor ring resonator R1 with an output fed to the photodetector (PD) 1. The light in the second waveguide is coupled to a reference ring resonator R2 with an output to the photodetector (PD) 2. FIG. 63B shows an example power spectra 6308 of the sensing elements and the frequency discriminator 6106 over time. The fringe pattern of the discriminator 6106 with the known free-spectral range may enable extraction of resonance wavelength shift due to the presence of analyte.

The optical source 6102 may be tuned across the resonances of the two identical ring resonators R1, R2 and an unbalanced MZI (UMZI)-based frequency discriminator 6106. A microfluidic channel may be employed to flow the to-be-sensed analyte on the sensor ring R1. The refractive index change resulting from the presence of the analyte on the surface of the sensor ring R1 may result in a relative shift of the resonance wavelengths between the two rings R1 and R2. This shift may be detected by PD 1 and 2, as illustrated in FIG. 63B. The resulting wavelength shift may scale with the components (e.g., biomarkers) and concentration of the analyte and may be extracted from the detector outputs.

The optical source 6102 in the sensor system may be a distributed feedback laser (DFB), a (sampled grating) distributed Bragg reflector laser (DBR laser), a vertical-cavity semiconductor emitting laser (VCSEL), a Vernier-tuned (VT) DBR laser, coupled ring-resonator laser (CRR), or any other laser diode configuration that is tunable thermally, electrically, mechanically, etc. across the ring resonances. The sensor system may account for the nonlinear tuning dynamics of the optical source 6102 (e.g., by using the output of an UMZI that has a known free-spectral range (FSR)). The relative movement of the output frequency of the source 6102 may then be evaluated (e.g., by using the spacing between the output fringes of the UMZI as shown in FIG. 63C).

The choice of the optical source 6102 may be determined by the required wavelength resolution for sensing, the material platform of the passive components, and/or the sampling rate of the read-out electronics 6112. The frequency drift of the optical source 6102 (e.g., laser) caused by the inherent white and flicker frequency noise components may lower the achievable wavelength resolution in the sampling period while the required relative-intensity-noise and the output power of the laser may be determined by the dynamic range of the electronics and the extinction ratio of the sensor element.

The optical splitter network 6104 depicted in FIG. 61 may be realized using any number of coupling systems (e.g., binary tree of directional couplers, multi-mode-interference couplers, etc.). The required flatness of the splitting ratio across the tuning range of the optical source may be determined by the thermal and/or nonlinear effects of the ring resonators or other sensor elements employed in the system. Improved compactness of the splitter network 6104 may be realized through implementation of a series of 1×N splitters comprising any number of coupling systems and/or coupler configurations including those described above. The optical splitter network 6104 can take any form and may be a combination of switches, wavelength multiplexing, and so on.

The frequency discriminator depicted in FIG. 61 may be used to evaluate the relative wavelength movement of the tuned optical source 6102. While the example of the discriminator depicted in FIG. 61 utilizes an unbalanced Mach-Zehnder interferometer (UMZI) configuration, other devices such as a stable Fabry-Perot cavity, a ring resonator, a gas cell, a free-space etalon, or any other reference cavity with a known free-spectral range and higher degree of thermal stability may be employed for clocking the read-out electronics as the source wavelength is tuned across the sensing element. In some examples, only the relative movement of source wavelength is of interest and the knowledge of absolute wavelength of source is not required, assuming the material and waveguide dispersion of the sensing element do not significantly vary the group index over the anticipated wavelength drift of the source and reference during the time of measurement.

### Robotic Biosensor Systems

Robotic pipettors and similar machines have become widely available. Current automated liquid handling systems are most often used on simple pipetting workflows like well-plating, serial dilutions, etc. Incorporating automated liquid handling systems into more complicated chemical and biological processes is of significant interest. Further, to enhance these systems, an inexpensive, flexible, highly multiplexed manner of sensing performed reactions is beneficial. The present disclosure discusses a system in which a robotic chemistry and biology platform is integrated with integrated photonic sensors. This includes systems and methods for coupling automated systems with a photonics-based sensing platform to perform chemical and biochemical assays. Silicon photonics-based biosensors could be beneficial over currently used analytical methods for use in rapid, point-of-care medical diagnosis and other bioassays.

Example systems and methods related to automated testing and handling of test samples for a silicon photonics-based sensing platform are described herein. The biosensors can include the binding to or cleaving from various components to an optical component, and determining (e.g., measuring) a change in the optical response of the system. In one non-limiting example, a waveguide (e.g., associated with a ring resonator) is functionalized to immobilize reporter probes (e.g., RNA strands). These reporter probes may be linked to an optically active component (e.g., plasmonic nanoparticle, quantum dot, molecule, etc.) to enhance their optical effect on the waveguide. Next, a cleaving component (e.g., a CRISPR enzyme) that may interact with the reporter probes and a sensing target of interest (e.g., virus RNA or DNA) may be combined with analyte carrying the sensing target. Once activated, the cleaving component may cleave the reporter probes from the waveguide, resulting in an optically detectable signal (e.g., due to a refractive index shift). For example, the presence of these cleaved optically active components may be detected downstream from the waveguide using various spectroscopic techniques.

These processes may be further performed with or without common techniques associated with biosensing (e.g., target amplification). Other known techniques for facilitating interactions between the optical component and sensing targets, reporter probes, biological markers, pathogen, etc. (e.g., toehold switch) may be implemented in addition to or as an alternative to the described techniques.

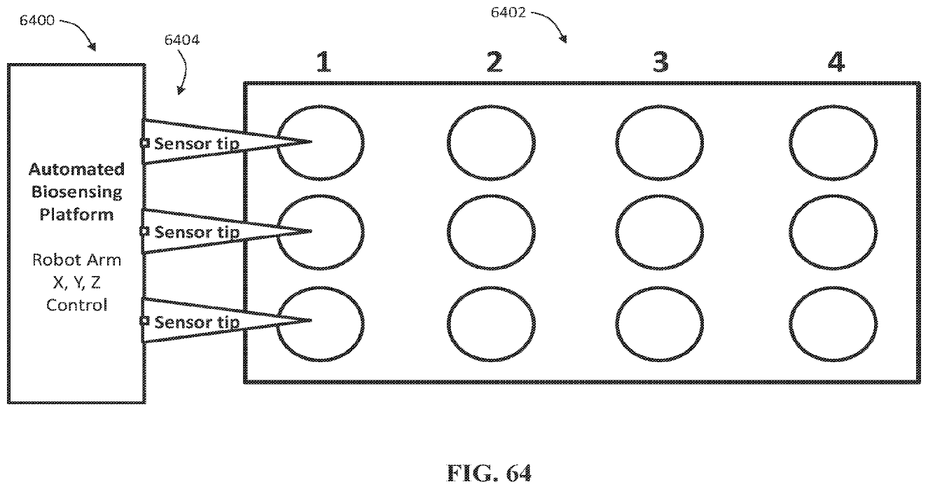

FIG. 64 illustrates an example implementation of an automated biosensing system 6400 capable of multiple tests within a multi-well plate 6402 (or a single test in a single well plate). Individual biological test samples may be placed in individual wells of the multi-well plate 6402. The system 6400 can include multiple sensor tips 6404 including disposable sensors that are connected to reusable sensing components. The automated system 6400 may test multiple well plate test samples simultaneously (or near simultaneously) by placing the biosensor-based tips directly into the well plate 6402.

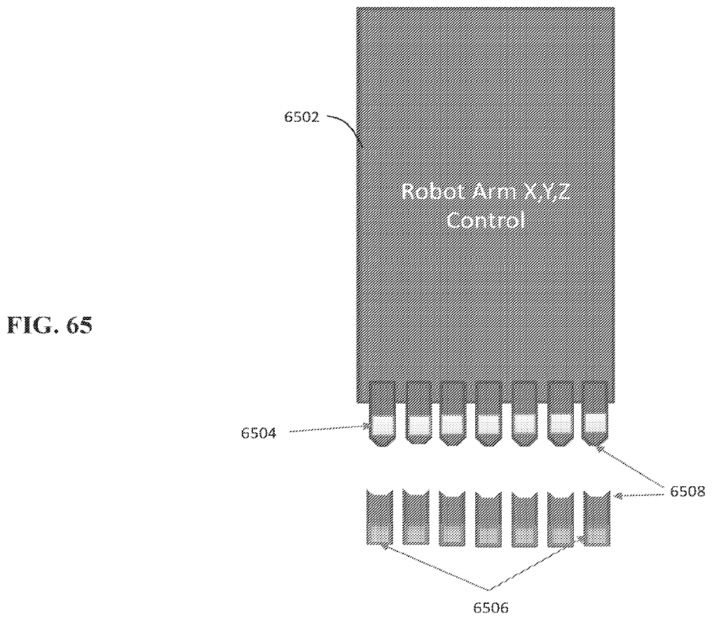

FIG. 65 illustrates mechanical alignment between a robot arm 6502 and a disposable sensor tip to enable optical communication between the reusable arm having reusable optical components 6504 and the disposable sensor chiplets 6506 on the sensor tips. A pluggable connector may connect the tip-based biosensor to reusable photonic chips or optical fibers within the automated biosensing platform. In some cases, there may be mechanical alignment features 6508 on the disposable tips that enable precise optical alignment between the sensor chips 6506 and the optics 6504 on the robot arm 6502. For example, the optics 6504 can include optical chip and/or fiber bundle (e.g., multi-fiber push on (MPO) connector). One potential advantage of this tip-based sensor and well-plate combination is the reduction of components in the biosensing platform. For example, instead of moving fluid around within the sensor via microfluidics, the tip-based sensors could be moved (via the robotic arm) to different wells containing different solutions e.g., reference samples, test samples, sensor regeneration solutions, etc.).

The system may include one or more tunable lasers, single wavelength lasers, or broadband light sources that is coupled to the disposable sensors via a photonic chip and/or a fiber bundle (e.g., an MPO connector). The system may include switching optics such that light can be directed to different disposable sensors serially to increase intensity.

In some examples, the disposable tip-based biosensing chip may include multiple optical components (e.g., waveguides or optical fibers) that can be used to multiplex different tests (e.g., immunoassays, viral RNA/DNA, etc.) on the same test sample (e.g., in the same well). Redundant testing (e.g., for the same virus) may increase sensitivity and/or specificity, while multiplexing tests for multiple pathogens (e.g., COVID-19 and flu) and/or multiple patient samples may be advantageous for facile widespread testing. Coupled automated liquid handling could permit these redundant and/or multiplexed tests to run more precisely and efficiently.

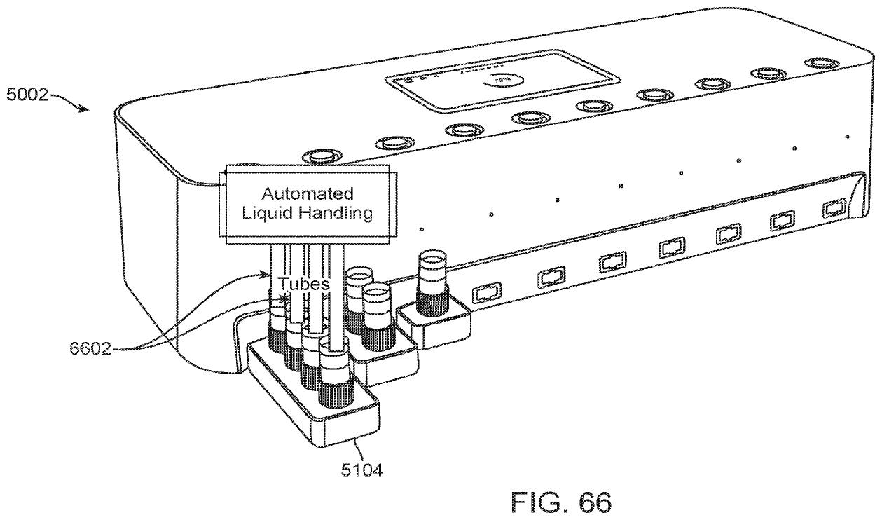

Coupling automated liquid handling within the biosensing platform (e.g., via a robotic pipetting robot) may improve the accuracy and throughput of the photonics-based biosensing platform. FIG. 66 illustrates an example implementation of automated liquid handling (e.g., pipetting) to insert sample tests directly into the disposable sensors coupled to a tabletop biosensing platform 5002. In this example, tubes 6602 that carry sample test analyte may be injected periodically into one or more input ports of disposable test cartridges 4704 (e.g. having one or more waveguide-based tests as described above) that are inserted into a tabletop biosensing platform 5002.

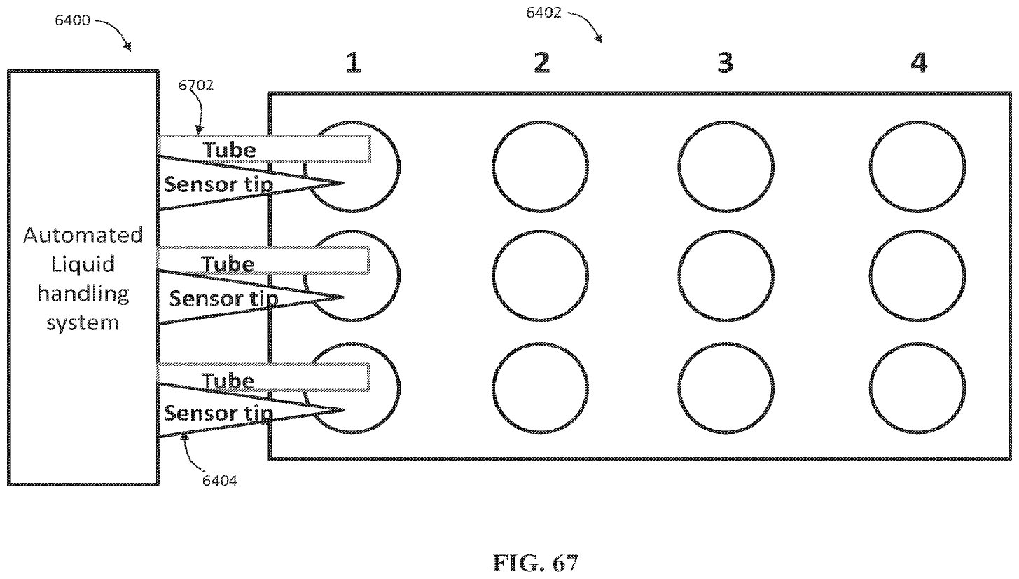

FIG. 67 illustrates an example implementation of an automated liquid handling system to draw samples into tubes and flow them over sensor chips via microfluidic channels. In this implementation, tubes 6702 may be coupled to a microfluidic system and the sensors as part of the automated liquid handling system 6400. These tubes 6702 can be placed into wells, e.g., in a 96-well plate 6402, and a robotic arm may be used to draw up reagents and/or samples. Suction may be provided by a peristaltic pump or syringe pump, or another source of negative pressure. In order to keep the samples from evaporating or interacting with the ambient air, a foil seal may be placed on the wells and a sharp point may be added to the tube 6702 or tube holder in order to pierce the foil and give the tube access to the sample.

In some cases, the automated liquid handling system may contain modules for thermocycling, PCR, heat blocks, fluorometers, shakers/mixers, chillers and other modules relevant for performing general biochemistry.

Using automated liquid handling may improve precision and accuracy of injection volumes. In some cases, accurate and/or precise sample analyte volumes may improve the quantification of the biosensing target concentration. Additionally or alternatively, liquid handling may improve accuracy and/or precision of injection timing of sample analyte into the biosensing platform. This may also improve biosensing target concentration quantification and overall test accuracy.

For example, automated liquid handling may enable pooling test samples in series (e.g., samples are introduced to the biosensing platform at regular time intervals until a positive test is recorded). In particular, accurate and/or precise injection timing and volumes would be crucial in narrowing down the positive test sample. This may be performed using a disposable microfluidic cartridge that contains the sensor chip such that it can be portable and used in the field.

In some examples, a device may be added to the system in which the disposable sensor tips can be placed such that the user can pipette (by hand or via automated liquid handling) samples into wells that then feed into droplet ejectors which apply drops to one or more sensors on the sensor chip withing the disposable sensor tip. This may enable multiplexed functionalization of the chip such that when it is used downstream in reactions, each sensor has been functionalized with a different protein or chemical. Each sensor chip design may include a matching functionalization cartridge design that couples it to the wells that are filled by the user. Thus, the user may add drops accurately to the surface as the cartridge and sensor chip holder are mechanically aligned to one another via alignment structures.

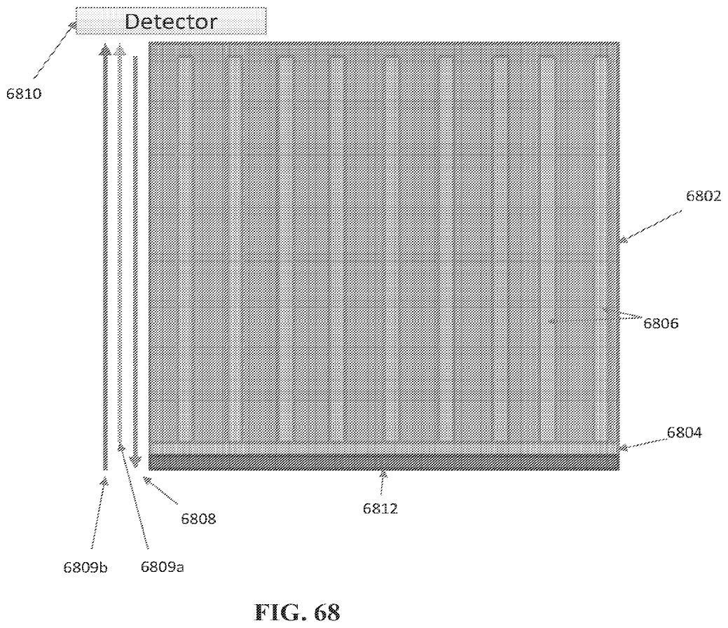

FIG. 68 illustrates a sensor chip with edge facets of waveguides used for biolayer interferometry. The sensor tips may include photonic chips where the waveguides terminating at the facet of the chip 6802 are coated with a thin layer of oxide 6804 or other material that has a different refractive index than the waveguide(s) 6806. The thin layer (e.g., oxide layer 6804) may be functionalized to facilitate photonic biosensing (e.g., via biolayer interferometry). For example, reflections of incoming coherent light 6808 may emerge from the interface between the waveguide and thin layer (e.g., silicon-oxide interface) and the oxide-biolayer-water interface. These reflections (e.g., reflection from first layer 6809a, reflection from second layer 6809b, etc.) may form interference patterns that can be measured via a detector 6810 configured to collect the back-reflections and/or record interference pattern. This may result in detector interference pattern or spectrum that shifts as the refractive index of the biolayer 6812 changes with binding/unbinding/cleavage of biomolecules.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described components and systems can generally be integrated together in a single device or system or packaged into multiple devices or systems.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

### Terminology

The phraseology and terminology used herein is for the purpose of description and should not be regarded as limiting.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (hut for use of the ordinal term), to distinguish the claim elements.

What is claimed is:

1. An integrated photonic system for biosensing, the system comprising:
a cartridge comprising a sensor photonic integrated subcircuit, the cartridge configured to receive a biological sample;
an interrogator photonic circuit optically coupled to the cartridge and comprising:
(i) a light source configured to generate light; and
(ii) one or more waveguides configured to carry the light,
wherein the light is used to determine a characteristic of the biological sample in the cartridge.

2. The system of claim 1, further comprising:
a stage configured to removably engage the cartridge and facilitate alignment of a light path of the interrogator photonic circuit and a light path of the cartridge.

3. The system of claim 2, wherein the stage comprises an ultrasound or a sound generator for at least one of: (i) preventing non-specific binding or (ii) mixing.

4. The system of claim 1, wherein the stage comprises at least one of: (a) a thermoelectric heater or (b) a thermoelectric cooler.

5. The system of claim 1, further comprising:
an isolation window disposed between the cartridge and the interrogator photonic circuit and configured to:
(a) physically isolate the interrogator photonic circuit from the biological sample, and
(b) enable the light to pass between the interrogator photonic circuit and the cartridge.

6. The system of claim 1, further comprising:
an alignment module configured to facilitate alignment between a light path of the cartridge and a light path of the interrogator photonic circuit.

7. The system of claim 6, wherein the alignment module actively facilitates alignment between the light path of the cartridge and the light path of the interrogator photonic circuit.

8. The system of claim 6, wherein the alignment module passively facilitates alignment between the light path of the cartridge and the light path of the interrogator photonic circuit.

9. The system of claim 6, wherein the alignment module enables an optical coupling efficiency greater than 10%.

10. The system of claim 6, further comprising:
an indicator coupled to the alignment module and configured to display a signal indicating whether the cartridge is aligned to the interrogator photonic circuit.

11. The system of claim 6, further comprising:
at least one lens configured to focus light between the interrogator photonic circuit and the cartridge.

12. The system of claim 1, wherein the interrogator photonic circuit comprises a control circuit configured to control the light.

13. The system of claim 12, wherein the control circuit comprises a detection circuit configured to detect the light.

14. The system of claim 12, wherein the light source is edge-coupled to the control circuit.

15. The system of claim 12, wherein the light source is coupled to the control circuit via an optical fiber.

16. The system of claim 1, wherein the cartridge comprises a microfluidic cell.

17. The system of claim 16, wherein the microfluidic cell comprises at least one of: (a) a magnetic microstirrer, (b) a plasmonic vortex mixer, or (c) a flow-inducing device.

18. The system of claim 17, wherein the microfluidic cell comprises the magnetic microstirrer, and wherein the system further comprises a stage configured to removably engage the cartridge and facilitate alignment of a light path of the interrogator photonic circuit and a light path of the cartridge, the stage comprising a transmitter configured to power the magnetic microstirrer.

19. The system of claim 17, wherein the flow-inducing device is an absorptive pad or a microfluidic capillary pump.

20. The system of claim 1, wherein the microfluidic cell comprises at least one of: (i) a protein, (ii) a reagent, or (iii) a rinsing fluid.

21. The system of claim 1, wherein the microfluidic cell comprises at least one microfluidic channel, a wall of the channel having an amplifier enzyme attached thereto.

22. The system of claim 1, wherein the stage is configured to receive a plurality of cartridges.

23. The system of claim 1, further comprising:
a splitter coupled to the light source; and
a frequency discriminator coupled to the splitter and configured to determine a change in a wavelength of the light source.

24. The system of claim 23, wherein the frequency discriminator comprises an unbalanced Mach-Zehnder interferometer (MZI), a Fabry-Perot cavity, a ring resonator, a gas cell, or a free-space etalon.

25. The system of claim 23, wherein the frequency discriminator comprises at least one of silicon, silica, or silicon nitride.

26. The system of claim 1, wherein the light source is tunable thermally, electrically, and/or mechanically.

27. The system of claim 1, further comprising:
a robotic device coupled to the interrogator photonic circuit and configured to position the cartridge to contact the biological sample.

28. The system of claim 27, wherein the robotic device is configured to discard the cartridge.

29. The system of claim 27, wherein the robotic device is configured to replace the cartridge automatically.

30. A method for biosensing, the method comprising:
obtaining a biological sample in a cartridge, wherein the cartridge comprises a sensor photonic integrated subcircuit;
positioning the cartridge relative to an interrogator photonic circuit such that the cartridge is optically coupled with the interrogator photonic circuit, wherein the interrogator photonic circuit comprises (i) a light source configured to generate light, (ii) a waveguide configured to carry the light, and iii) a photodetector configured to detect said light after passing through said waveguides; and
determining, via the light, a characteristic of the biological sample in the cartridge.

31. The method of claim 30, further comprising:
determining, via an alignment module, whether the cartridge is optically coupled with the interrogator photonic circuit.

32. The method of claim 31, further comprising:
determining a coupling efficiency between the cartridge and the interrogator.

33. The method of claim 30, wherein said characteristic of the biological sample is determined based on a change in resonance, interference, or absorption caused by the biological sample.

34. The method of claim 30, wherein said waveguide is optically coupled to a probe.

35. The method of claim 34, wherein said probe binds specifically to a target biomolecule in said sample.

36. The method of claim 35, wherein said probe is an antibody, an antigen, or an aptamer.

37. The method of claim 35, wherein said target biomolecules is bound by a detection antibody.

38. The method of claim 37, wherein said detection antibody comprises an optically active component.

39. The method of claim 34, wherein a component of said biological sample initiates a cleavage of said probe.

40. The method of claim 39, wherein said probe comprises an optically active component.

41. The method of claim 38 or claim 40, wherein said optically active component is a plasmonic nanoparticle, a gold nanoparticle, a quantum dot, or a fluorophore.

42. The method of claim 39, wherein said probe comprises a silicon particle.

43. The method of claim 39, wherein said probe comprises a magnetic particle.

44. The method of claim 43, wherein said magnetic particle comprises iron-oxide.

44. The method of claim 30, wherein said waveguide comprises an optical ring resonator or an unbalanced Mach-Zehnder interferometer.

45. The method of claim 39, wherein said component of said biological sample activates a cleaving component.

46. The method of claim 41, wherein said component of said biological sample binds to a hairpin RNA encoding a cleaving component, wherein said binding facilitates translation of said RNA to generate said cleaving component.

47. The method of claim 41, wherein said cleaving component is a CRISPR enzyme.

48. The method of claim 30, wherein said cartridge further comprises an electromagnet.

49. The method of claim 30, wherein a target biomolecule of said sample is functionalized with a magnetic particle.

50. A method for detecting a target biomolecule in a biological sample, comprising
providing a device comprising a sensor functionalized with a probe, wherein said probe can be cleaved by a cleavage enzyme;
adding said biological sample to said device, wherein the presence of said target biomolecule results in generation of or activation of said cleavage enzyme;
detecting cleavage of said probe by said cleavage enzyme, thereby detecting the presence of said target biomolecule in said biological sample.

51. The method of claim 50, wherein said cleavage enzyme is a CRISPR complex.

52. The method of claim 51, wherein said CRISPR complex is a Cas12 complex or a Cas13 complex.

53. The method of claim 50, wherein said target biomolecule is RNA or DNA.

54. The method of claim 53, wherein said target biomolecule binds to a hairpin RNA encoding said cleavage enzyme, wherein said binding facilitates translation of said hairpin RNA to generate said cleavage enzyme.

55. The method of claim 50, wherein said sensor is an electrical sensor, an optical sensor, or a combination thereof.

56. The method of claim 50, wherein said sensor comprises a ring resonator or a Mach-Zehnder interferometer.

57. The method of claim 50, wherein said probe comprises an optically active component.

58. The method of claim 57, wherein said optically active component is a plasmonic nanoparticle, a gold nanoparticle, a quantum dot, or a fluorophore.

59. The method of claim 50, wherein said probe comprises a silicon particle.

60. The method of claim 50, wherein said probe comprises a magnetic particle.

61. The method of claim 60, wherein said magnetic particle comprises iron-oxide.
